(19) United States
(12) Patent Application Publication
(10) Pub. No.: US 2023/0000624 A1
(43) Pub. Date: Jan. 5, 2023

(54) DELIVERY SYSTEM CONFIGURATIONS

(71) Applicant: Edwards Lifesciences Corporation, Irvine, CA (US)

(72) Inventors: Hiroshi Okabe, Costa Mesa, CA (US); Hieu Minh Luong, Westminster, CA (US); Julio Cesar Sanchez, Garden Grove, CA (US); Jesse Robert Edwards, Silverado, CA (US); Alexander H. Cooper, Costa Mesa, CA (US); David Robert Landon, Huntington Beach, CA (US)

(21) Appl. No.: 17/940,868

(22) Filed: Sep. 8, 2022

Related U.S. Application Data
(63) Continuation of application No. PCT/US2021/023303, filed on Mar. 19, 2021.
(60) Provisional application No. 62/993,885, filed on Mar. 24, 2020.

(51) Int. Cl. A61F 2/24

(52) U.S. Cl. CPC A61F 2/2436; A61F 2/2439; A61F 2/9517

(57) ABSTRACT

Disclosed are embodiments of delivery systems for delivery of replacement heart valves. This can include mitral, aortic, tricuspid, and pulmonary valves. The delivery systems can include one or more different components and configurations that advantageously improve placement of the replacement heart valves during the operation of the delivery system.

### RELATED APPLICATIONS

This application is a continuation of International Application No. PCT/US2021/023303, filed Mar. 19, 2021, which designates the United States and was published in English by the International Bureau on Sep. 30, 2021 as WO2021/194899, which claims priority to U.S. Provisional App. No. 62/993,885, filed Mar. 24, 2020, the entire contents of each of which are hereby incorporated by reference.

### FIELD

Certain embodiments disclosed herein relate generally to prostheses for implantation within a lumen or body cavity and delivery systems for a prosthesis. In particular, the prostheses and delivery systems relate in some embodiments to replacement heart valves, such as replacement mitral heart valves or replacement tricuspid heart valves.

### BACKGROUND

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner.

Delivering a prosthesis to a desired location in the human body, for example delivering a replacement heart valve to the mitral valve, can also be challenging. Obtaining access to perform procedures in the heart or in other anatomical locations may require delivery of devices percutaneously through tortuous vasculature or through open or semi-open surgical procedures. The ability to control the deployment of the prosthesis at the desired location can also be challenging.

### SUMMARY

Embodiments of the present disclosure are directed to a delivery system, such as but not limited to a delivery system for a replacement heart valve. Further embodiments are directed to methods of use to deliver and/or controllably deploy a prosthesis, such as but not limited to a replacement heart valve, to a desired location within the body. In some embodiments, a replacement heart valve and methods for delivering a replacement heart valve to a native heart valve, such as a mitral valve, an aortic valve, or a tricuspid valve, are provided.

In some embodiments, a delivery system and method are provided for delivering a replacement heart valve to a native mitral valve location. The delivery system and method may utilize a transseptal approach. In some embodiments, components of the delivery system facilitate bending of the delivery system to steer a prosthesis from the septum to a location within the native mitral valve. In some embodiments, a capsule is provided for containing the prosthesis for delivery to the native mitral valve location. In other embodiments, the delivery system and method may be adapted for delivery of implants to locations other than the native mitral valve.

The present disclosure includes, but is not limited to, the following embodiments.

Embodiment 1: A delivery system for a replacement heart valve, the delivery system comprising:
an inner shaft having a proximal end and a distal end,
wherein the distal end of the inner shaft comprises a manifold,
wherein the manifold comprises a plurality of circumferentially-spaced apart apertures, each of the apertures having a tooth extending from a distal edge of the aperture toward a proximal edge of the aperture;
an outer shaft member configured to cover the apertures of the manifold when in a distal position to prevent release of the looped end of the attachment tether,
wherein the outer shaft member comprises at least one aperture configured to be aligned with the plurality of circumferentially-spaced apart apertures of the manifold;
at least one attachment tether configured to releasably connect to at least one eyelet of the replacement heart valve,
wherein a first looped end of the at least one attachment tether is configured to be attached to the distal end of the inner shaft,
wherein a second looped end of the at least one attachment tether is configured to extend through the at least one eyelet of the replacement heart valve, through the at least one aperture of the outer sleeve into a respective one of the apertures of the manifold, and then looped over a free proximal end of the tooth,
wherein at least one of the outer shaft member and the inner manifold is configured to translate axially relative to the other to uncover the second looped end of the at least one attachment tether such that the second looped end can be removed from the tooth, thereby facilitating release of the replacement heart valve from the delivery system.

Embodiment 2: The delivery system of Embodiment 1, wherein the at least one aperture of the outer shaft member comprises a plurality of circumferentially-spaced apart apertures.

Embodiment 3: The delivery system of Embodiment 1 or 2, wherein the outer shaft member is configured to be moved proximally relative to the inner shaft to uncover the second looped end of the at least one attachment tether.

Embodiment 4: The delivery system of Embodiment 1 or 2, wherein the inner shaft is configured to be moved distally relative to the outer shaft member to uncover the second looped end of the at least one attachment tether.

Embodiment 5: The delivery system of any of Embodiments 1-4, wherein the outer shaft member comprises a sleeve.

Embodiment 6: The delivery system of any of Embodiments 1-5, wherein the at least one attachment member comprises a plurality of attachment tethers, wherein a first looped end of each of the plurality of attachment tethers is configured to be attached to the distal end of the inner shaft, and wherein a second looped end of each of the plurality of attachment tethers is configured to configured to extend through the at least one eyelet of the replacement heart valve, through the at least one aperture of the outer sleeve into a respective one of the apertures of the manifold, and then looped over a free proximal end of the tooth of the respective one of the apertures of the manifold.

Embodiment 7: A method of facilitating delivery of a replacement heart valve within a body of a patient, the method comprising:
advancing a distal portion of a delivery system to a desired implantation location within a heart of the patient,
wherein the delivery system comprises an inner shaft and an outer shaft member,
wherein the distal end of the inner shaft comprises at least one radial aperture, the at least one radial aperture having a tooth extending from a distal edge of the at least one radial aperture toward a proximal edge of the at least one radial aperture,
wherein the outer shaft member comprises at least one radial aperture configured to be aligned with the at least one radial aperture of the inner shaft,
wherein a first looped end of an attachment tether is attached to a distal end of the inner shaft,
wherein a second looped end of the attachment tether is removably coupled to the tooth of the at least one radial aperture of the inner shaft after having been inserted through an eyelet of the replacement heart valve;
causing the outer shaft member and the inner shaft to transition from a locked configuration in which the second looped end of the attachment member cannot be removed from the tooth to an unlocked configuration in which the second looped end of the attachment member can be removed from the tooth, thereby decoupling the replacement heart valve from the delivery system and allowing the replacement heart valve to remain in the desired implantation location; and
removing the delivery system from the patient.

Embodiment 8: The method of Embodiment 7, wherein the desired implantation location is a native mitral valve.

Embodiment 9: The method of Embodiment 7, wherein the desired implantation location is a native tricuspid valve.

Embodiment 10: The method of any of Embodiments 7-9, wherein causing the outer shaft member and the inner shaft to transition from the locked configuration to the unlocked configuration comprises moving the inner shaft in a distal direction.

Embodiment 11: The method of any of embodiments 7-9, wherein causing the outer shaft member and the inner shaft to transition from the locked configuration to the unlocked configuration comprises moving the outer shaft member in a proximal direction.

Embodiment 12: A delivery system for a replacement heart valve, the delivery system comprising a shaft having a proximal end and a distal end, a manifold on a distal end of the shaft, wherein the manifold comprises a plurality of radially extending apertures, each aperture having a tooth, at least one attachment tether configured to releasably connect to the replacement heart valve, wherein looped portions of the at least one attachment tether extend through the radially extending apertures of the manifold to surround the tooth, and a sleeve configured to cover the radially extending apertures in a distal position to prevent release of the looped portions, wherein the sleeve is configured to be proximally translated to a proximal position to uncover the looped portions so that the replacement heart valve is released from the at least one attachment tether.

Embodiment 13: A delivery system for a replacement heart valve, the delivery system comprising:
a shaft having a proximal end and a distal end;
a manifold on a distal end of the shaft, wherein the manifold comprises a plurality of radially extending apertures;
at least one attachment tether configured to releasably connect to the replacement heart valve, wherein looped portions of the at least one attachment tether extend through the radially extending apertures of the manifold; and
a release tether configured to extend through the looped portions of the at least one attachment tether;
wherein when the release tether is withdrawn from the looped portions, the replacement heart valve is released from the at least one attachment tether.

Embodiment 14: The delivery system of Embodiment 13, wherein the manifold comprises an inner manifold and an outer manifold.

Embodiment 15: The delivery system of Embodiment 14, wherein the outer manifold comprises the radially extending apertures for receiving the looped portions of a distal end of the at least one attachment tether.

Embodiment 16: The delivery system of Embodiment 14 or 15, wherein a proximal end of the at least one attachment tether is attached to the inner manifold.

Embodiment 17: The delivery system of any one of Embodiments 13-15, wherein the at least one attachment tether comprises one and only one attachment tether.

Embodiment 18: The delivery system of any one of Embodiments 13-15, wherein the at least one attachment tether comprises a plurality of attachment tethers.

Embodiment 19: A handle for a replacement heart valve delivery system, the handle comprising, a housing, at least one knob located on the housing, at least one ring gear in communication with the at least one knob, wherein the at least one knob is configured to rotate the at least one ring gear, at least one planet gear located within the at least one ring gear and in communication with the at least one ring gear, wherein the at least one planet gear remains in the same circumferential position with relation to the at least one ring gear during rotation of the at least one ring gear, and a linear travel screw in communication with the at least one planet gear, wherein the linear travel screw is configured to move in an axial direction upon rotation of the at least one knob.

Embodiment 20: The handle of Embodiment 19, wherein the at least one ring gear comprises a plurality of planet gears, each planet gear of the plurality of planet gears in communication with one of a plurality of linear travel screws.

Embodiment 21: A delivery system for use with a replacement heart valve, the system comprising a bendable nose cone shaft having a proximal end and a distal end, a nose cone attached to a distal end of the nose cone shaft; and a rigid shaft at least partially covering the bendable nose cone shaft, wherein the rigid shaft is configured to axially translate with respect to the bendable nose cone shaft to cover or uncover the bendable nose cone shaft, wherein, when the bendable nose cone shaft is uncovered by the rigid shaft, the bendable nose cone shaft is configured to allow the replacement heart valve attached to the system to conform to a native anatomy of a native heart valve.

Embodiment 22: The delivery system of Embodiment 21, wherein the bendable nose cone shaft comprises a bendable polymer.

Embodiment 23: A delivery system for use with a replacement heart valve, the system comprising an inner retention member configured to releasably retain a replacement heart valve, and an outer retention member configured to at least partially cover a portion of the replacement heart valve and the inner retention member, wherein the outer retention member and the inner retention member are configured to have a locked and an unlocked configuration, wherein when in the unlocked configuration the outer retention member is configured to move axially with respect to the inner retention member, and when in the locked configuration the outer retention member is prevented from moving axially with respect to the inner retention member.

Embodiment 24: The delivery system of Embodiment 23, wherein the inner retention member comprises an outer threading and the outer retention member comprises an inner threading, and wherein the locked position occurs when the outer threading is threaded onto the inner threading.

Embodiment 25: The delivery system of Embodiment 23 or 24, wherein the inner retention member and outer retention member comprise locking features that an operator can unlock when the operator wants to move the outer retention member axially with respect to the inner retention member.

Embodiment 26: A delivery system for use with a replacement heart valve, the system comprising an inner shaft having a proximal end and a distal end, the inner shaft having a cut pattern to allow bending of the inner shaft, and a spine, an outer shaft surrounding the inner shaft and having a proximal end and a distal end, the outer shaft having a cut pattern to allow bending of the outer shaft, and a spine, and at least one pull wire configured to bend one or more of the inner shaft and the outer shaft, wherein one of the inner shaft and the outer shaft are configured to rotate with respect to one another between a flexing configuration and an unflexing configuration, wherein, in the flexing configuration, the cut pattern of the inner shaft is aligned with the cut pattern of the outer shaft, and wherein, in the unflexing configuration, the spine of the inner shaft is aligned with the cut pattern of the outer shaft.

Embodiment 27: The delivery system of Embodiment 26, wherein the cut pattern of the inner shaft is the same as the cut pattern of the outer shaft.

Embodiment 28: The delivery system of Embodiment 26, wherein the cut pattern of the inner shaft is different than the cut pattern of the outer shaft.

Embodiment 29: The delivery system of any of Embodiments 26-28, wherein the at least one pull wire comprises a plurality of pull wires, wherein a first pull wire is configured to cause bending of the inner shaft and wherein a second pull wire is configured to cause bending of the outer shaft.

Embodiment 30: A method of facilitating controlled bending of bendable coaxial shafts of a delivery system, the method comprising:
providing a delivery system comprising:
   an inner shaft having a proximal end and a distal end, the inner shaft having a cut pattern to allow bending of the inner shaft, and a spine;
   an outer shaft surrounding the inner shaft and having a proximal end and a distal end, the outer shaft having a cut pattern to allow bending of the outer shaft, and a spine; and
   at least one pull wire configured to bend one or more of the inner shaft and the outer shaft;
rotating one of the inner shaft and the outer shaft with respect to the other between a flexing configuration and an unflexing configuration;
wherein, in the flexing configuration, the cut pattern of the inner shaft is aligned with the cut pattern of the outer shaft to allow bending of the outer shaft upon actuation of the at least one pull wire; and
wherein, in the unflexing configuration, the spine of the inner shaft is aligned with the cut pattern of the outer shaft to prevent bending of the outer shaft upon actuation of the at least one pull wire.

Embodiment 31: The method of Embodiment 30, wherein the cut pattern of the inner shaft is the same as the cut pattern of the outer shaft.

Embodiment 32: The method of Embodiment 31, wherein the cut pattern of the inner shaft is different than the cut pattern of the outer shaft.

Embodiment 33: The method of any of Embodiments 30-32, wherein the at least one pull wire comprises a plurality of pull wires, wherein a first pull wire is configured to cause bending of the inner shaft and wherein a second pull wire is configured to cause bending of the outer shaft.

Embodiment 34: A delivery system for use with a replacement heart valve, the system comprising an inner shaft having a proximal end and a distal end, and an outer shaft surrounding the inner shaft and having a proximal end and a distal end, wherein the inner shaft and the outer shaft are keyed together at the distal end of the outer shaft and the distal end of the inner shaft to prevent rotation of the inner shaft with respect to the outer shaft.

Embodiment 35: The delivery system of Embodiment 34, wherein the inner shaft and the outer shaft each have an ovaloid cross-section.

Embodiment 36: The delivery system of Embodiment 34 or 35, wherein at least the distal end of the inner shaft comprises a locking tab and at least the distal end of the outer shaft comprises a notch or slot configured to receive the locking tab so as to prevent rotation of the inner shaft with respect to the outer shaft.

Embodiment 37: The delivery system of Embodiment 34 or 35, wherein at least the distal end of the outer shaft comprises a locking tab and at least the distal end of the inner shaft comprises a notch or slot configured to receive the locking tab so as to prevent rotation of the inner shaft with respect to the outer shaft.

Embodiment 38: The delivery system of any of Embodiment 34-37, wherein the outer shaft comprises an outer pull wire lumen and wherein the inner shaft comprises an inner pull wire lumen.

Embodiment 39: A delivery system for a replacement heart valve, the delivery system comprising an inner shaft having a proximal end and a distal end, a mid shaft surrounding the inner shaft and having a proximal end and a distal end and a lumen, the mid shaft having a disc on the distal end, the diameter of the disc being greater than a diameter of the inner shaft, wherein the disc comprises a longitudinally extending aperture radially outward of the lumen, and an outer shaft surrounding the mid shaft, the outer shaft comprising a radially extending aperture having a tooth, and an attachment tether configured to releasably connect to the replacement heart valve, wherein the attachment tether has a first end connected to the inner shaft, wherein the attachment tether extends through the lumen of the mid shaft and out the distal end of the mid shaft, and wherein the attachment tether extends proximally through the longitudinally extending aperture and attaches to the tooth of the outer shaft, wherein, when the disc is proximally translated it prevents release of the attachment tether from the tooth, and wherein, when the disc is distally translated it releases the tether from the tooth so that the replacement heart valve is released.

Embodiment 40: A delivery system for a replacement heart valve, the delivery system comprising a capsule configured to surround the replacement heart valve and configured to radially compress the replacement heart valve, wherein the capsule comprises a distal portion, wherein the distal portion comprises a hypotube with an interrupted spiral cut pattern, an outer polymer jacket configured to at least partially cover a radially outwards surface of the hypotube, a fluoropolymer liner on a radially inner surface of the hypotube, and a porous fluoropolymer outer coating on one of the hypotube or the outer polymer jacket, and a proximal portion having a smaller diameter than the distal portion, wherein the proximal portion comprises a hypotube with a cut pattern, the hypotube surrounded by an outer polymer jacket.

Embodiment 41: The delivery system of Embodiment 40, wherein the outer polymer jacket of the distal portion covers greater than 90% of a length of the hypotube of the distal portion.

Embodiment 42: The delivery system of Embodiment 40 or 41, wherein the hypotube of the distal portion is configured to provide compression resistance and the liner and/or outer polymer jacket of the distal portion is configured to provide tension resistance.

Embodiment 43: The delivery system of any of Embodiments 40-42, wherein the liner of the distal portion is porous.

Embodiment 44: The delivery system of any of Embodiments 40-43, wherein the liner of the distal portion is bonded to a metal structure of the hypotube of the distal portion using a reflow process.

Embodiment 45: A handle for a replacement heart valve delivery system, the handle comprising:
a housing comprising a threaded portion;
at least one adapter; and
at least one knob located on the housing, the at least one knob comprising:
   a first portion configured to be coupled to the at least one adapter, and
   a second portion configured to be detachably coupled to the first portion, wherein the first and second portions are configured to detach when a threshold force is exerted on the at least one adapter.

Embodiment 46: The handle of Embodiment 45, wherein the first portion comprises a recess and an internal spring within the recess.

Embodiment 47: The handle of Embodiment 46, wherein the second portion comprises a projection configured to be received by the recess of the first portion.

Embodiment 48: The handle of Embodiment 47, wherein the internal spring of the first portion releasably retains the projection of the second portion when the first and second portions are coupled.

Embodiment 49: The handle of Embodiment 45, wherein the first portion comprises a hole and the second portion comprises a spring tab, the hole of the first portion being configured to receive the spring tab of the second portion when the first and second portions are coupled.

Embodiment 50: The handle of Embodiment 49, wherein the spring tab of the second portion is configured to deflect from the hole of the first portion when the threshold force is exerted on the at least one adapter to decouple the first and second portions.

Embodiment 51: The handle of Embodiment 45, wherein the first portion comprises a ramped recess and the second portion comprises a ramped projection, the ramped recess of the first portion being configured to receive the ramped projection of the second portion when the first and second portions are coupled.

Embodiment 52: The handle of Embodiment 51, wherein the ramped projection of the second portion is configured to deflect from the ramped recess of the first portion when the threshold force is exerted on the at least one adapter to decouple the first and second portions.

Embodiment 53: The handle of Embodiment 45, wherein the first portion comprises a recess and the second portion comprises a pin, the recess of the first portion being configured to receive the pin of the second portion when the first and second portions are coupled.

Embodiment 54: The handle of Embodiment 53, wherein the pin of the second portion is configured to break from the second portion when the threshold force is exerted on the at least one adapter to decouple the first and second portions.

Embodiment 55: The handle of Embodiment 45, wherein the first portion comprises a recess and the second portion comprises a spring plunger, the recess of the first portion being configured to receive a portion of the spring plunger of the second portion when the first and second portions are coupled.

Embodiment 56: The handle of Embodiment 55, wherein the portion of the spring plunger of the second portion is configured to decouple from the ramped recess of the first portion when the threshold force is exerted on the at least one adapter to decouple the first and second portions.

Embodiment 57: The handle of any of Embodiments 45-56, wherein the second portion is configured to engage with the threaded portion.

Embodiment 58: A handle for a replacement heart valve delivery system, the handle comprising:
a housing comprising a threaded portion;
at least one adapter; and
at least one knob located on the housing, the at least one knob comprising:
   a first portion configured to be coupled to the at least one adapter, and
   a second portion coupled to the first portion, wherein a distance between the first portion and the second portion is correlated with a force exerted on the at least one adapter.

Embodiment 59: The handle of Embodiment 58, wherein the at least one knob further comprises a connector configured to extend between and couple the first and second portions, the connector comprising a distal portion and a proximal portion.

Embodiment 60: The handle of Embodiment 59, wherein the first portion is configured to receive the distal portion of the connector and the second portion is configured to receive the proximal portion of the connector.

Embodiment 61: The handle of Embodiment 59 or 60, wherein the connector comprises one or more indicators.

Embodiment 62: The handle of Embodiment 61, wherein the one or more indicators indicate the force exerted on the adapter.

Embodiment 63: The handle of any of Embodiments 58-62, wherein the distance between the first and second portions increases as the force exerted on the at least one adapter increases.

Embodiment 64: The handle of any of Embodiments 58-63, wherein the distance between the first and second portions decreases as the force exerted on the at least one adapter decreases.

Embodiment 65: The handle of any of Embodiments 58-64, wherein the first and second portions are configured to detach when the force exerted on the adapter reaches a threshold force.

Embodiment 66: The handle of any of Embodiments 58-65, wherein the second portion is configured to engage with the threaded portion.

### BRIEF DESCRIPTION OF THE DRAWINGS

FIG. 1 shows an embodiment of a delivery system.

FIG. 2A shows a partial cross-sectional view of the distal end of the delivery system of FIG. 1 loaded with the valve prosthesis of FIG. 3A.

FIG. 2B shows a partial cross-sectional view of the distal end of the delivery system of FIG. 1 without the valve prosthesis of FIG. 3A.

FIG. 2C shows a partial cross-sectional view of the distal end of the delivery system of FIG. 1 without with certain shaft assemblies translated along the rail assembly.

FIG. 3A shows a side view of an embodiment of a valve prosthesis that may be delivered using the delivery systems described herein.

FIG. 3B shows a side view of an embodiment of an aortic valve prosthesis that may be delivered using the delivery systems described herein.

FIG. 4 shows a perspective view of the distal end of the delivery system of FIG. 1.

FIG. 5 show components of the delivery system of FIG. 4 with the outer sheath assembly moved proximally and out of view.

FIG. 6A show components of the delivery system of FIG. 5 with the mid shaft assembly moved proximally and out of view.

FIG. 6B illustrates a cross-section of the rail assembly.

FIG. 7 show components of the delivery system of FIG. 6A with the rail assembly moved proximally and out of view.

FIG. 8 show components of the delivery system of FIG. 7 with the inner assembly moved proximally and out of view.

FIGS. 9A and 9B illustrate embodiments of a guide wire shield.

FIG. 10 illustrates an embodiment of an outer hypotube.

FIG. 11 illustrates an embodiment of a mid shaft hypotube.

FIG. 12A illustrates an embodiment of the mid shaft hypotube of FIG. 11 as a flat pattern.

FIG. 12B illustrates an embodiment of an outer retention ring.

FIG. 13 illustrates an embodiment of a rail assembly.

FIG. 14 illustrates an embodiment of an inner assembly.

FIG. 15 illustrates a cross-section of a capsule.

FIG. 16 illustrates an embodiment of a delivery system handle.

FIG. 17 illustrates a cross-section of the delivery system handle of FIG. 16.

FIG. 18 illustrates a schematic representation of a transseptal delivery approach.

FIG. 19 illustrates a schematic representation of a valve prosthesis positioned within a native mitral valve.

FIG. 20 shows the valve prosthesis frame located within a heart.

FIGS. 21-23 show steps of a method for delivery of the valve prosthesis to an anatomical location.

FIGS. 24A-B illustrate the methodology of the rail delivery system.

FIGS. 25A-25B illustrate embodiments of a planetary gear design.

FIGS. 26A-26B illustrates embodiments of a flexible shaft and stiffened sheath.

FIG. 27 illustrates an embodiment of threaded components.

FIGS. 28A-28C illustrate bendable aligned shafts.

FIGS. 29A-29C illustrate embodiments of disclosed rotatable shafts.

FIGS. 30, 31, 31A and 31B illustrate unkeyed (FIG. 30) and keyed (FIGS. 31, 31A and 31B) shaft configurations.

FIGS. 32A-36B illustrate embodiments of tethering release mechanisms.

FIGS. 37-39B illustrate an embodiment of a capsule construction.

FIG. 40 illustrates another embodiment of a delivery system handle with an embodiment of a failsafe knob.

FIG. 41A illustrates a cross-section of the failsafe knob of FIG. 40 in a first configuration.

FIG. 41B illustrates a cross-section of the failsafe knob of FIG. 40 in a second configuration.

FIGS. 41C-41F illustrate schematic illustrations of example connection mechanisms.

FIG. 42A illustrates a cross-section of an embodiment of an indicator knob in a first configuration.

FIG. 42B illustrates a cross-section of the indicator knob of FIG. 42A in a second configuration.

### DETAILED DESCRIPTION

The present specification and drawings provide aspects and features of the disclosure in the context of several embodiments of replacement heart valves, delivery systems and methods that are configured for use in the vasculature of a patient, such as for replacement of natural heart valves in a patient. These embodiments may be discussed in connection with replacing specific valves such as the patient's aortic, tricuspid, or mitral valve. However, it is to be understood that the features and concepts discussed herein can be applied to products other than heart valve implants. For example, the controlled positioning, deployment, and securing features described herein can be applied to medical implants, for example other types of expandable prostheses, for use elsewhere in the body, such as within an artery, a vein, or other body cavities or locations. In addition, particular features of a valve, delivery system, etc. should not be taken as limiting, and features of any one embodiment discussed herein can be combined with features of other embodiments as desired and when appropriate. While certain of the embodiments described herein are described in connection with a transfemoral delivery approach, it should be understood that these embodiments can be used for other delivery approaches such as, for example, transapical or transjugular approaches. Moreover, it should be understood that certain of the features described in connection with some embodiments can be incorporated with other embodiments, including those which are described in connection with different delivery approaches.

### Delivery System

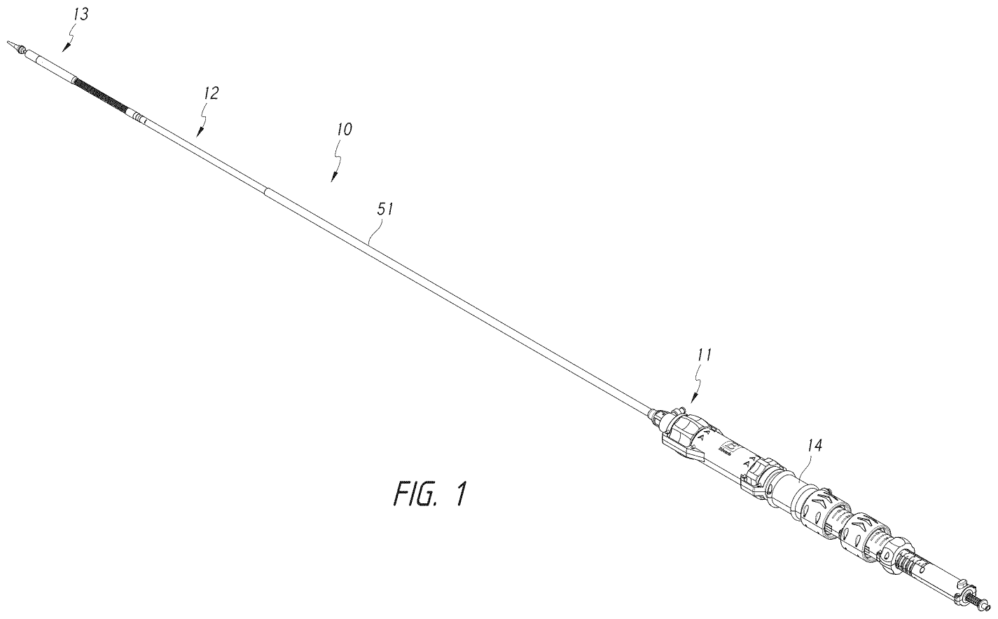

FIG. 1 illustrates an embodiment of a delivery device, system, or assembly 10. The delivery system 10 can be used to deploy a prosthesis, such as a replacement heart valve, within the body. In some embodiments, the delivery system 10 can use a dual plane deflection approach to properly delivery the prosthesis. Replacement heart valves can be delivered to a patient's heart mitral or tricuspid valve annulus or other heart valve location in various manners, such as by open surgery, minimally-invasive surgery, and percutaneous or transcatheter delivery through the patient's vasculature. Example transfemoral approaches may be found in U.S. Pat. Pub. No. 2015/0238315, filed Feb. 20, 2015, the entirety of which is hereby incorporated by reference in its entirety. While the delivery system 10 is described in connection with a percutaneous delivery approach, and more specifically a transfemoral delivery approach, it should be understood that features of delivery system 10 can be applied to other delivery system, including delivery systems for a transapical delivery approach.

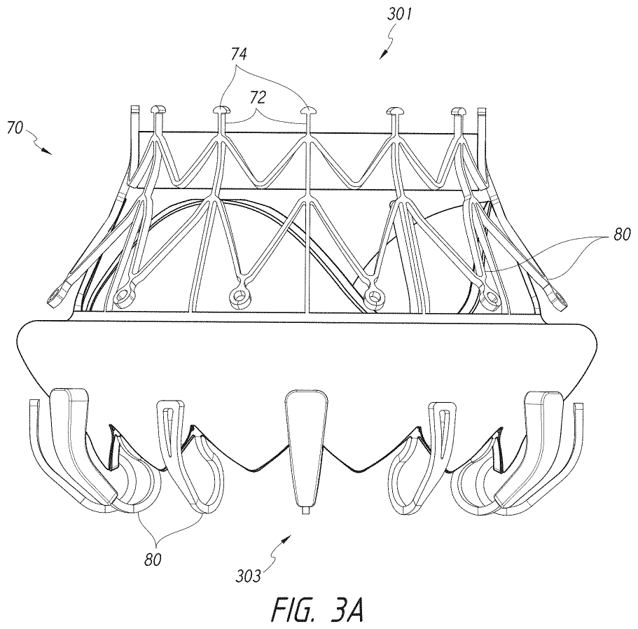

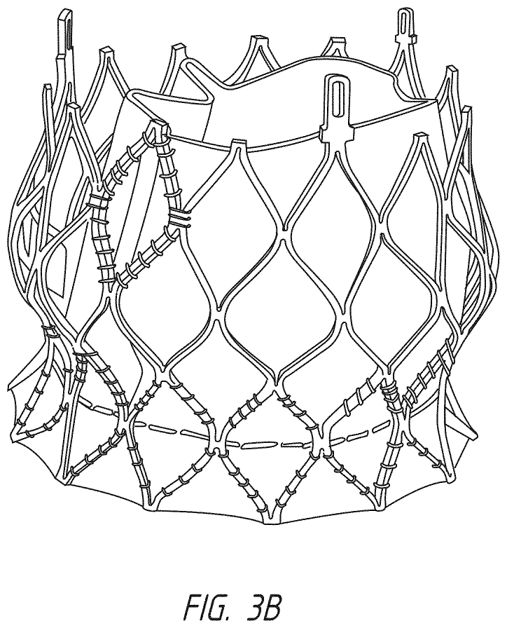

The delivery system 10 can be used to deploy a prosthesis, such as a replacement heart valve as described elsewhere in this specification, within the body. The delivery system 10 can receive and/or cover portions of the prosthesis such as a first end 301 and second end 303 of the prosthesis 70 illustrated in FIG. 3A below. For example, the delivery system 10 may be used to deliver an expandable implant or prosthesis 70, where the prosthesis 70 includes the first end 301 and the second end 303, and wherein the second 303 end is configured to be deployed or expanded before the first end 301.

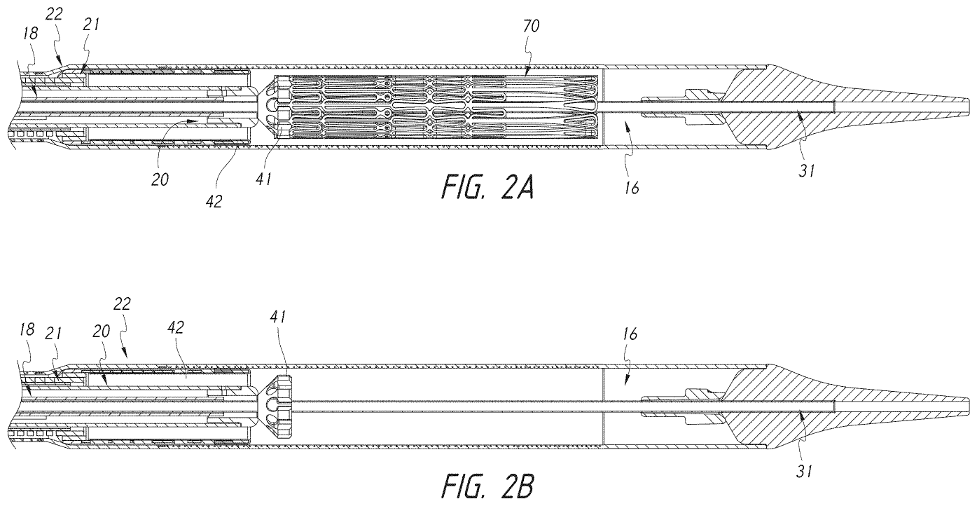

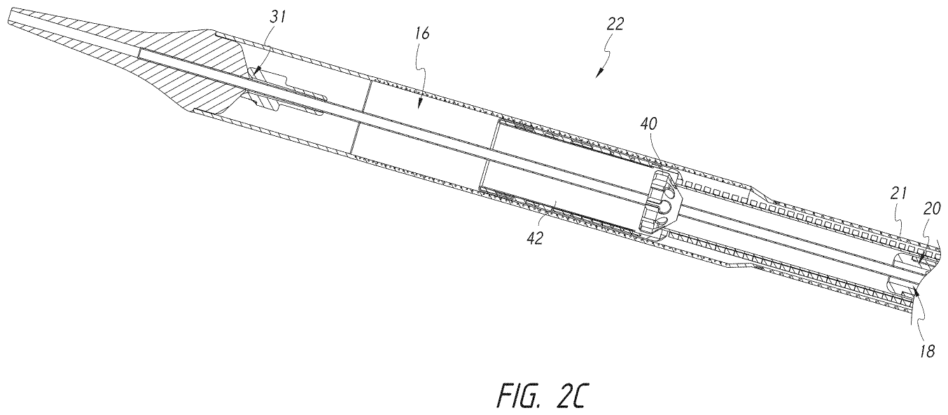

FIG. 2A further shows an example of the prosthesis 70 that can be inserted into the delivery system 10, specifically into the implant retention area 16. For ease of understanding, in FIG. 2A, the prosthesis is shown with only the bare metal frame illustrated. The implant or prosthesis 70 can take any number of different forms. A particular example of frame for a prosthesis is shown in FIG. 3A, though it will be understood that other designs can also be used. The prosthesis 70 can include one or more sets of anchors, such as distal (or ventricular) anchors 80 extending proximally when the prosthesis frame is in an expanded configuration and proximal (or atrial) anchors 82 extending distally when the prosthesis frame is in an expanded configuration. The prosthesis can further include struts 72 which may end in mushroom-shaped tabs 74 at the first end 301. Further discussion can be found in U.S. Publication No. 2015/0328000A1, published Nov. 19, 2015, hereby incorporated by reference in its entirety.

In some embodiments, the delivery system 10 can be used in conjunction with a replacement aortic valve, such as shown in FIG. 3B. In some embodiments the delivery system 10 can be modified to support and delivery the replacement aortic valve. However, the procedures and structures discussed below can similarly be used for a replacement mitral, tricuspid, or aortic valve.

Additional details and example designs for a prosthesis are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, and 2018/0055629, the entirety of these patents and publications are hereby incorporated by reference and made a part of this specification. Further details and embodiments of a replacement heart valve or prosthesis and its method of implantation are described in U.S. Publication Nos. 2015/0328000 and 2016/0317301 the entirety of each of which is hereby incorporated by reference and made a part of this specification.

The delivery system 10 can be relatively flexible. In some embodiments, the delivery system 10 is particularly suitable for delivering a replacement heart valve to a mitral valve location through a transseptal approach (e.g., between the right atrium and left atrium via a transseptal puncture).

As shown in FIG. 1, the delivery system 10 can include a shaft assembly 12 comprising a proximal end 11 and a distal end 13, wherein a handle 14 is coupled to the proximal end of the assembly 12. The shaft assembly 12 can be used to hold the prosthesis for advancement of the same through the vasculature to a treatment location. The delivery system 10 can further comprise a relatively rigid live-on (or integrated) sheath 51 surrounding the shaft assembly 12 that can prevent unwanted motion of the shaft assembly 12. The live-on sheath 51 can be attached at a proximal end of the shaft assembly 12 proximal to the handle 14, for example at a sheath hub. The shaft assembly 12 can include an implant retention area 16 (shown in FIGS. 2A-B with FIG. 2A showing the prosthesis 70 and FIG. 2B with the prosthesis 70 removed) at its distal end that can be used for this purpose. In some embodiments, the shaft assembly 12 can hold an expandable prosthesis in a compressed state at implant retention area 16 for advancement of the prosthesis 70 within the body. The shaft assembly 12 may then be used to allow controlled expansion of the prosthesis 70 at the treatment location. In some embodiments, the shaft assembly 12 may be used to allow for sequential controlled expansion of the prosthesis 70 as discussed in detail below. The implant retention area 16 is shown in FIGS. 2A-B at the distal end of the delivery system 10, but may also be at other locations. In some embodiments, the prosthesis 70 may be rotated in the implant retention area 16, such as through the rotation of the inner shaft assembly 18 discussed herein.

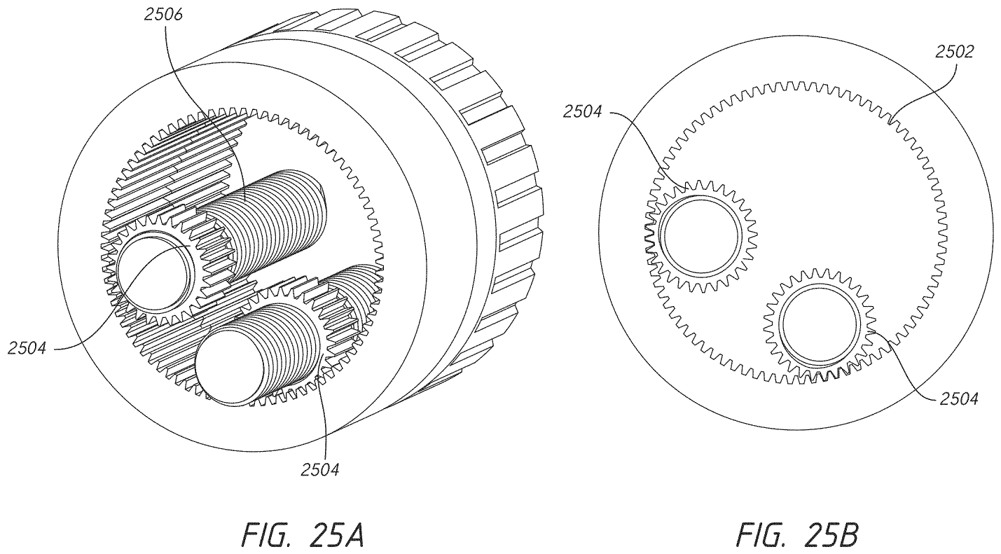

As shown in cross-sectional view of FIGS. 2A-B, the distal end of the delivery system 10 can include one or more subassemblies such as an outer sheath assembly 22, a mid shaft assembly 21, a rail assembly 20, an inner shaft assembly 18, and a nose cone assembly 31 as will be described in more detail below. In some embodiments, the delivery system 10 may not have all of the assemblies disclosed herein. For example, in some embodiments a full mid shaft assembly may not be incorporated into the delivery system 10, such as described in the embodiment of FIGS. 25-36 below. In some embodiments, the assemblies disclosed below may be in a different radial order than is discussed.

In particular, embodiments of the disclosed delivery system 10 can utilize a steerable rail in the rail assembly 20 for steering the distal end of the delivery system 10, allowing the implant to be properly located in a patient's body. As discussed in detail below, the steerable rail can be, for example, a rail shaft that extends through the delivery system 10 from the handle 14 generally to the distal end. In some embodiments, the steerable rail has a distal end that ends proximal to the implant retention area 16. A user can manipulate the bending of the distal end of the rail, thereby bending the rail in a particular direction. In preferred embodiments, the rail has more than one bend along its length, thereby providing multiple directions of bending. As the rail is bent, it presses against the other assemblies to bend them as well, and thus the other assemblies of the delivery system 10 can be configured to steer along with the rail as a cooperating single unit, thus providing for full steerability of the distal end of the delivery system.

Once the rail is steered into a particular location in a patient's body, the prosthesis 70 can be advanced along or relative to the rail through the movement of the other sheaths/shafts relative to the rail and released into the body. For example, the rail can be bent into a desired position within the body, such as to direct the prosthesis 70 towards the native mitral valve. The other assemblies (e.g., the outer sheath assembly 22, the mid shaft assembly 21, the inner assembly 18, and the nose cone assembly 31) can passively follow the bends of the rail. Further, the other assemblies (e.g., the outer sheath assembly 22, the mid shaft assembly 21, the inner assembly 18, and the nose cone assembly 31) can be advanced together (e.g., relatively together, sequentially with one actuator, simultaneously, almost simultaneously, at the same time, closely at the same time) relative to the rail while maintaining the prosthesis 70 in the compressed position without releasing or expanding the prosthesis 70 (e.g., within the implant retention area 16). The other assemblies (e.g., the outer sheath assembly 22, the mid shaft assembly 21, the inner assembly 18, and the nose cone assembly 31) can be advanced distally or proximally together relative to the rail. In some embodiments, only the outer sheath assembly 22, mid shaft assembly 21, and inner assembly 18 are advanced together over the rail. Thus, the nose cone assembly 31 may remain in the same position. The assemblies can be individually, sequentially, or simultaneously, translated relative to the inner assembly 18 in order to release the implant 70 from the implant retention area 16.

FIG. 2C illustrates the sheath assemblies, specifically the outer sheath assembly 22, the mid shaft assembly 21, the inner shaft assembly 18, and the nose cone assembly 31 having translated distally together along the rail assembly 20, further details on the assemblies are below. In some embodiments, the outer sheath assembly 22, the mid shaft assembly 21, the inner shaft assembly 18, and the nose cone assembly 31 translate together (e.g., relatively together, sequentially with one actuator, simultaneously, almost simultaneously, at the same time, closely at the same time). This distal translation can occur while the implant 70 remains in a compressed configuration within the implant retention area 16.

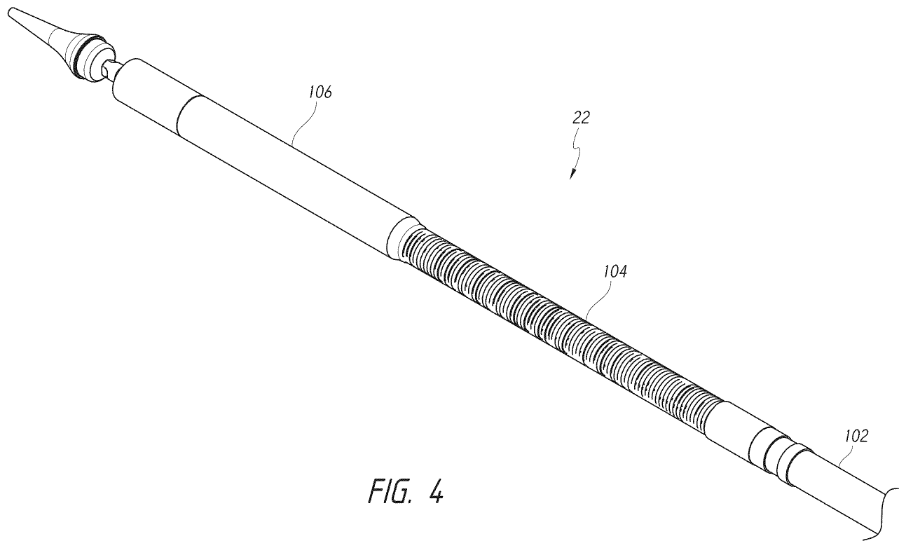

As shown in FIGS. 2A-2C and as further shown in FIGS. 4-8, starting with the outermost assembly, the delivery system can include an outer sheath assembly 22 forming a radially outer covering, or sheath, to surround an implant retention area 16 and prevent the implant from radially expanding. Specifically, the outer sheath assembly 22 can prevent radial expansion of the distal end of the implant from radially expanding. Moving radially inward, the mid shaft assembly 21 can be composed of a mid shaft hypotube 43 with its distal end attached to an outer retention member or outer retention ring 42 for radially retaining a portion of the prosthesis in a compacted configuration, such as a proximal end of the prosthesis 70. The mid shaft assembly 21 can be located within a lumen of the outer sheath assembly 22. Moving further inwards, the rail assembly 20 can be configured for steerability, as mentioned above and further described below. The rail assembly 20 can be located within a lumen of the mid shaft assembly 21. Moving further inwards, the inner shaft assembly 18 can be composed of an inner shaft with its distal end attached to inner retention member or inner retention ring 40 (such as a PEEK ring) for axially retaining the prosthesis, for example the proximal end of the prosthesis. The inner shaft assembly 18 can be located within a lumen of the rail assembly 20. Further, the most radially-inward assembly is the nose cone assembly 31 which includes the nose cone shaft 27 having its distal end connected to the nose cone 28. The nose cone 28 can have a tapered tip. The nose cone assembly 31 is preferably located within a lumen of the inner shaft assembly 18. The nose cone assembly 31 can include a lumen for a guide wire to pass therethrough.

The shaft assembly 12, and more specifically the nose cone assembly 31, inner assembly 18, rail assembly 20, mid shaft assembly 21, and outer sheath assembly 22, can be collectively configured to deliver a prosthesis 70 positioned within the implant retention area 16 (shown in FIG. 2A) to a treatment location. One or more of the subassemblies can then be moved to allow the prosthesis 70 to be released at the treatment location. For example, one or more of the subassemblies may be movable with respect to one or more of the other subassemblies. The handle 14 can include various control mechanisms that can be used to control the movement of the various subassemblies as will also be described in more detail below. In this way, the prosthesis 70 can be controllably loaded onto the delivery system 10 and then later deployed within the body. Further, the handle 14 can provide steering to the rail assembly 20, providing for bending/flexing/steering of the distal end of the delivery system 10.

As will be discussed below, the inner retention member 40, the outer retention ring 42, and the outer sheath assembly 22 can cooperate to hold the prosthesis 70 in a compacted configuration. The inner retention member 40 is shown engaging struts 72 at the proximal end 301 of the prosthesis 70 in FIG. 2A. For example, slots located between radially extending teeth on the inner retention member 40 can receive and engage the struts 72 which may end in mushroom-shaped tabs 74 on the proximal end of the prosthesis 70. The mid shaft assembly 21 can be positioned over the inner retention member 40 so that the first end 301 of the prosthesis 70 is trapped between the inner retention member 40 and the outer retention ring 42, thereby securely attaching it to the delivery system 10 between the mid shaft assembly 21 and the inner retention member 40. The outer sheath assembly 22 can be positioned to cover the second end 303 of the prosthesis 70.

The outer retention member 42 may be attached to a distal end of the mid shaft hypotube 43 which can in turn be attached to a proximal tube 44 at a proximal end, which in turn can be attached at a proximal end to the handle 14. The outer retention member 42 can provide further stability to the prosthesis 70 when in the compressed position. The outer retention member 42 can be positioned over the inner retention member 40 so that the proximal end of the prosthesis 70 is trapped therebetween, securely attaching it to the delivery system 10. The outer retention member 42 can encircle a portion of the prosthesis 70, in particular the first end 301, thus preventing the prosthesis 70 from expanding. Further, the mid shaft assembly 21 can be translated proximally with respect to the inner assembly 18 into the outer sheath assembly 22, thus exposing a first end 301 of the prosthesis 70 held within the outer retention member 42. In this way the outer retention member 42 can be used to help secure a prosthesis 70 to or release it from the delivery system 10. The outer retention member 42 can have a cylindrical or elongate tubular shape, and may be referred to as an outer retention ring, though the particular shape is not limiting.

The mid shaft hypotube 43 itself can be made of, for example, high density polyethylene (HDPE), as well as other appropriate materials as described herein. The mid shaft hypotube 43 can be formed of a longitudinally pre-compressed HDPE tube, which can provide certain benefits. For example, the pre-compressed HDPE tube can apply a force distally onto the outer retention member 42, thus preventing accidental, inadvertent, and/or premature release of the prosthesis 70. Specifically, the distal force by the mid shaft hypotube 43 keeps the distal end of the outer retention member 42 distal to the inner retention member 40, thus preventing the outer retention member 42 from moving proximal to the inner retention member 40 before it is desired by a user to release the prosthesis 70. This can remain true even when the delivery system 10 is bent/deflected at a sharp angle. Further disclosure for the outer retention member 42 and mid shaft hypotube 43 can be found in U.S. Pat. Pub. No. 2016/0317301, hereby incorporated by reference in its entirety.

As shown in FIG. 2A, the distal anchors 80 can be located in a delivered configuration where the distal anchors 80 point generally distally (as illustrated, axially away from the main body of the prosthesis frame and away from the handle of the delivery system). The distal anchors 80 can be restrained in this delivered configuration by the outer sheath assembly 22. Accordingly, when the outer sheath 22 is withdrawn proximally, the distal anchors 80 can flip positions (e.g., bend approximately 180 degrees) to a deployed configuration (e.g., pointing generally proximally). FIG. 2A also shows the proximal anchors 82 extending distally in their delivered configuration within the outer sheath assembly 22. In other embodiments, the distal anchors 80 can be held to point generally proximally in the delivered configuration and compressed against the body of the prosthesis frame.

The delivery system 10 may be provided to users with a prosthesis 70 preinstalled. In other embodiments, the prosthesis 70 can be loaded onto the delivery system shortly before use, such as by a physician or nurse.

### Delivery System Assemblies

FIGS. 4-8 illustrate further views of delivery system 10 with different assemblies translated proximally and described in detail.

Starting with the outermost assembly shown in FIG. 4, the outer sheath assembly 22 can include an outer proximal shaft 102 directly attached to the handle 14 at its proximal end and an outer hypotube 104 attached at its distal end. A capsule 106 can then be attached generally at the distal end of the outer hypotube 104. In some embodiments, the capsule 106 can be 28 French or less in size. These components of the outer sheath assembly 22 can form a lumen for the other subassemblies to pass through.

The outer proximal shaft 102 may be a tube and is preferably formed of a plastic, but could also be a metal hypotube or other material. The outer hypotube 104 can be a metal hypotube which in some embodiments may be cut or have slots, as discussed in detail below. The outer hypotube 104 can be covered or encapsulated with a layer of ePTFE, PTFE, or other polymer/material so that the outer surface of the outer hypotube 104 is generally smooth.

A capsule 106 can be located at a distal end of the outer proximal shaft 102. The capsule 106 can be a tube formed of a plastic or metal material. In some embodiments, the capsule 106 is formed of ePTFE or PTFE. In some embodiments, this capsule 106 is relatively thick to prevent tearing and to help maintain a self-expanding implant in a compacted configuration. In some embodiments the material of the capsule 106 is the same material as the coating on the outer hypotube 104. As shown, the capsule 106 can have a diameter larger than the outer hypotube 104, though in some embodiments the capsule 106 may have a similar diameter as the hypotube 104. In some embodiments, the capsule 106 may include a larger diameter distal portion and a smaller diameter proximal portion. In some embodiments, there may be a step or a taper between the two portions. The capsule 106 can be configured to retain the prosthesis 70 in the compressed position within the capsule 106. Further construction details of the capsule 106 are discussed below.

The outer sheath assembly 22 is configured to be individually slidable with respect to the other assemblies. Further, the outer sheath assembly 22 can slide distally and proximally relative to the rail assembly 22 together with the mid shaft assembly 21, inner assembly 18, and nose cone assembly 31.

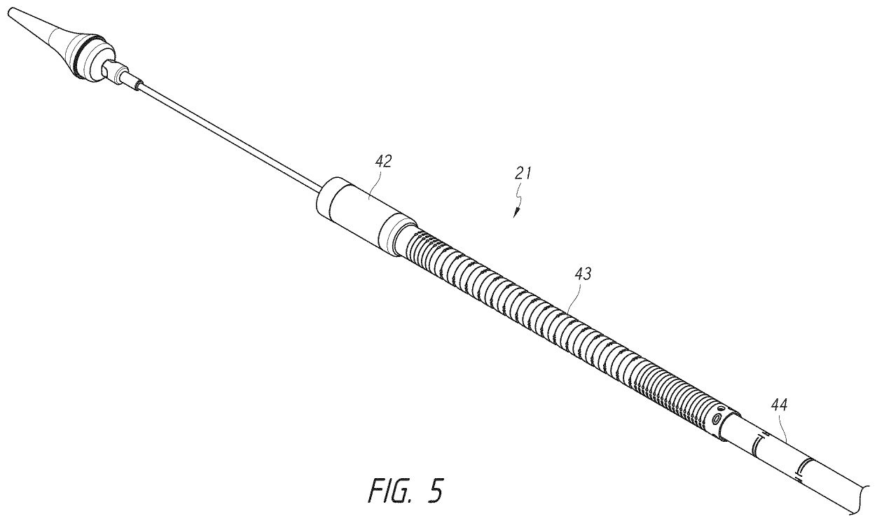

Moving radially inwardly, the next assembly is the mid shaft assembly 21. FIG. 5 shows a similar view as FIG. 4, but with the outer sheath assembly 22 removed, thereby exposing the mid shaft assembly 21.

The mid shaft assembly 21 can include a mid shaft hypotube 43 generally attached at its proximal end to a mid shaft proximal tube 44, which in turn can be attached at its proximal end to the handle 14, and an outer retention ring 42 located at the distal end of the mid shaft hypotube 43. Thus, the outer retention ring 42 can be attached generally at the distal end of the mid shaft hypotube 43. These components of the mid shaft assembly 21 can form a lumen for other subassemblies to pass through.

Similar to the other assemblies, the mid shaft hypotube 43 and/or mid shaft proximal tube 44 can comprise a tube, such as a hypodermic tube or hypotube (not shown). The tubes can be made from one of any number of different materials including Nitinol, stainless steel, and medical grade plastics. The tubes can be a single piece tube or multiple pieces connected together. Using a tube made of multiple pieces can allow the tube to provide different characteristics along different sections of the tube, such as rigidity and flexibility. The mid shaft hypotube 43 can be a metal hypotube which in some embodiments may be cut or have slots as discussed in detail below. The mid shaft hypotube 43 can be covered or encapsulated with a layer of ePTFE, PTFE, or other material so that the outer surface of the mid shaft hypotube 43 is generally smooth.

The outer retention ring 42 can be configured as a prosthesis retention mechanism that can be used to engage with the prosthesis 70, as discussed with respect to FIG. 2A. For example, the outer retention ring 42 may be a ring or covering that is configured to radially cover the struts 72 on the prosthesis 70. The outer retention ring 42 can also be considered to be part of the implant retention area 16, and may be at the proximal end of the implant retention area 16. With struts or other parts of a prosthesis 70 engaged with the inner retention member 40, discussed below the outer retention ring 42 can cover both the prosthesis 70 and the inner retention member 40 to secure the prosthesis 70 on the delivery system 10. Thus, the prosthesis 70 can be sandwiched between the inner retention member 40 of the inner shaft assembly 18 and the outer retention ring 42 of the mid shaft assembly 21.

The mid shaft assembly 21 is disposed so as to be individually slidable with respect to the other assemblies. Further, mid shaft assembly 21 can slide distally and proximally relative to the rail assembly 22 together with the outer sheath assembly 22, mid inner assembly 18, and nose cone assembly 31.

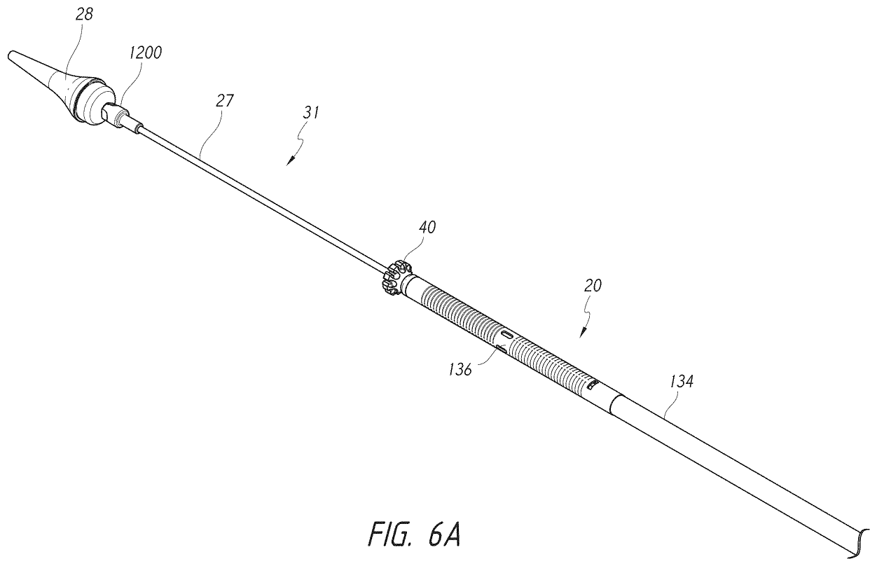

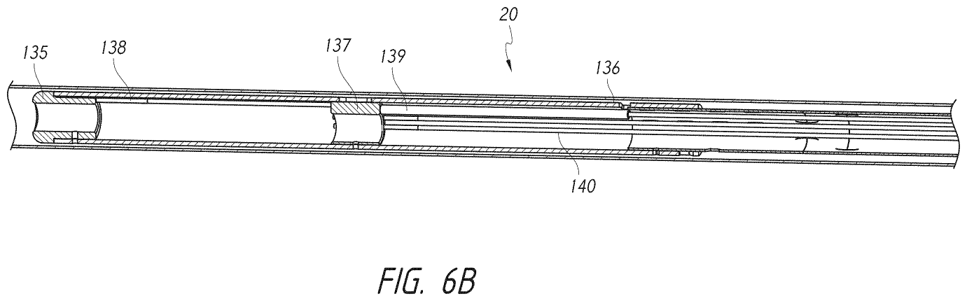

Next, radially inwardly of the mid shaft assembly 21 is the rail assembly 20. FIG. 6A shows approximately the same view as FIG. 5, but with the mid shaft assembly 21 removed, thereby exposing the rail assembly 20. FIG. 6B further shows a cross-section of the rail assembly 20 to view the pull wires. The rail assembly 20 can include a rail shaft 132 (or rail) generally attached at its proximal end to the handle 14. The rail shaft 132 can be made up of a rail proximal shaft 134 directly attached to the handle at a proximal end and a rail hypotube 136 attached to the distal end of the rail proximal shaft 134. The rail hypotube 136 can further include an atraumatic rail tip at its distal end. Furth, the distal end of the rail hypotube 136 can abut a proximal end of the inner retention member 40, as shown in FIG. 6. In some embodiments, the distal end of the rail hypotube 136 can be spaced away from the inner retention member 40. These components of the rail shaft assembly 20 can form a lumen for the other subassemblies to pass through.

As shown in FIG. 6B, attached to an inner surface of the rail hypotube 136 are one or more pull wires which can be used apply forces to the rail hypotube 136 and steer the rail assembly 20. The pull wires can extend distally from the knobs in the handle 14, discussed below, to the rail hypotube 136. In some embodiments, pull wires can be attached at different longitudinal locations on the rail hypotube 136, thus providing for multiple bending locations in the rail hypotube 136, allowing for multidimensional steering.

In some embodiments, a distal pull wire 138 can extend to a distal section of the rail hypotube 136 and two proximal pull wires 140 can extend to a proximal section of the rail hypotube 136, however, other numbers of pull wires can be used, and the particular amount of pull wires is not limiting. For example, a two pull wires can extend to a distal location and a single pull wire can extend to a proximal location. In some embodiments, ring-like structures attached inside the rail hypotube 136, known as pull wire connectors, can be used as attachment locations for the pull wires, such as proximal ring 137 and distal ring 135. In some embodiments, the rail assembly 20 can include a distal pull wire connector 135 and a proximal pull wire connector 139. In some embodiments, the pull wires can directly connect to an inner surface of the rail hypotube 136.

The distal pull wire 138 can be connected (either on its own or through a connector 135) generally at the distal end of the rail hypotube 136. The proximal pull wires 140 can connect (either on its own or through a connector 137) at a location approximately one quarter, one third, or one half of the length up the rail hypotube 136 from the proximal end. In some embodiments, the distal pull wire 138 can pass through a small diameter pull wire lumen 139 (e.g., tube, hypotube, cylinder) attached on the inside of the rail hypotube 136. This can prevent the wires 138 from pulling on the rail hypotube 136 at a location proximal to the distal connection. Further, the lumen 139 can act as compression coils to strengthen the proximal portion of the rail hypotube 136 and prevent unwanted bending. Thus, in some embodiments the lumen 139 is only located on the proximal half of the rail hypotube 136. In some embodiments, multiple lumens 139, such as spaced longitudinally apart or adjacent, can be used per distal wire 139. In some embodiments, a single lumen 139 is used per distal wire 139. In some embodiments, the lumen 139 can extend into the distal half of the rail hypotube 136. In some embodiments, the lumen 139 is attached on an outer surface of the rail hypotube 136. In some embodiments, the lumen 139 is not used.

For the pair of proximal pull wires 140, the wires can be spaced approximately 180° from one another to allow for steering in both directions. Similarly, if a pair of distal pull wires 138 is used, the wires can be spaced approximately 180° from one another to allow for steering in both directions. In some embodiments, the pair of distal pull wires 138 and the pair of proximal pull wires 140 can be spaced approximately 90° from each other. In some embodiments, the pair of distal pull wires 138 and the pair of proximal pull wires 140 can be spaced approximately 0° from each other. However, other locations for the pull wires can be used as well, and the particular location of the pull wires is not limiting. In some embodiments, the distal pull wire 138 can pass through a lumen 139 attached within the lumen of the rail hypotube 136. This can prevent an axial force on the distal pull wire 138 from creating a bend in a proximal section of the rail hypotube 136.

The rail assembly 20 is disposed so as to be slidable over the inner shaft assembly 18 and the nose cone assembly 31. In some embodiments, the outer sheath assembly 22, the mid shaft assembly 21, the inner shaft assembly 22, and the nose cone assembly 31 can be configured to slide together along or relative to the rail assembly 20, such as proximally and distally with or without any bending of the rail assembly 20. In some embodiments, the outer sheath assembly 22, the mid shaft assembly 21, the inner shaft assembly 22, and the nose cone assembly 31 can be configured to retain the implant 70 in a compressed position when they are simultaneously slid along or relative to the rail assembly 20.

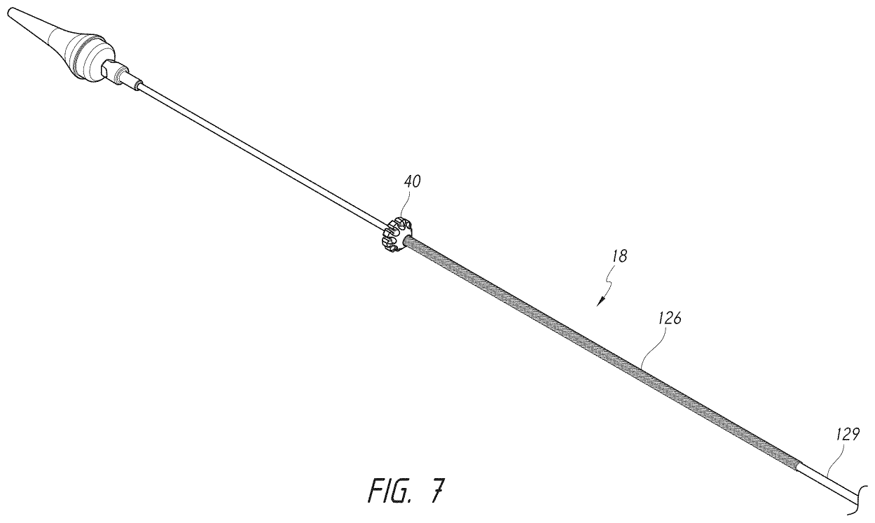

Moving radially inwards, the next assembly is the inner shaft assembly 18. FIG. 7 shows approximately the same view as FIG. 6A, but with the rail assembly 20 removed, thereby exposing the inner shaft assembly 18.

The inner shaft assembly 18 can include an inner shaft 122 generally attached at its proximal end to the handle 14, and an inner retention ring 40 located at the distal end of the inner shaft 122. The inner shaft 122 itself can be made up of an inner proximal shaft 124 directly attached to the handle 14 at a proximal end and a distal section 126 attached to the distal end of the inner proximal shaft 124. Thus, the inner retention ring 40 can be attached generally at the distal end of the distal section 126. These components of the inner shaft assembly 18 can form a lumen for the other subassemblies to pass through.

Similar to the other assemblies, the inner proximal shaft 124 can comprise a tube, such as a hypodermic tube or hypotube (not shown). The tube can be made from one of any number of different materials including Nitinol, cobalt chromium, stainless steel, and medical grade plastics. The tube can be a single piece tube or multiple pieces connected together. A tube comprising multiple pieces can provide different characteristics along different sections of the tube, such as rigidity and flexibility. The distal section 126 can be a metal hypotube which in some embodiments may be cut or have slots as discussed in detail below. The distal section 126 can be covered or encapsulated with a layer of ePTFE, PTFE, or other material so that the outer surface of the distal section 126 is generally smooth.

The inner retention member 40 can be configured as a prosthesis retention mechanism that can be used to engage with the prosthesis 70, as discussed with respect to FIG. 2A. For example, the inner retention member 40 may be a ring and can include a plurality of slots configured to engage with struts 72 on the prosthesis 70. The inner retention member 40 can also be considered to be part of the implant retention area 16, and may be at the proximal end of the implant retention area 16. With struts or other parts of a prosthesis 70 engaged with the inner retention member 40, the outer retention ring 42 can cover both the prosthesis and the inner retention member 40 to secure the prosthesis on the delivery system 10. Thus, the prosthesis 70 can be sandwiched between the inner retention member 40 of the inner shaft assembly 18 and the outer retention ring 42 of the mid shaft assembly 21.

The inner shaft assembly 18 is disposed so as to be individually slidable with respect to the other assemblies. Further, the inner assembly 18 can slide distally and proximally relative to the rail assembly 22 together with the outer sheath assembly 22, mid shaft assembly 21, and nose cone assembly 31.

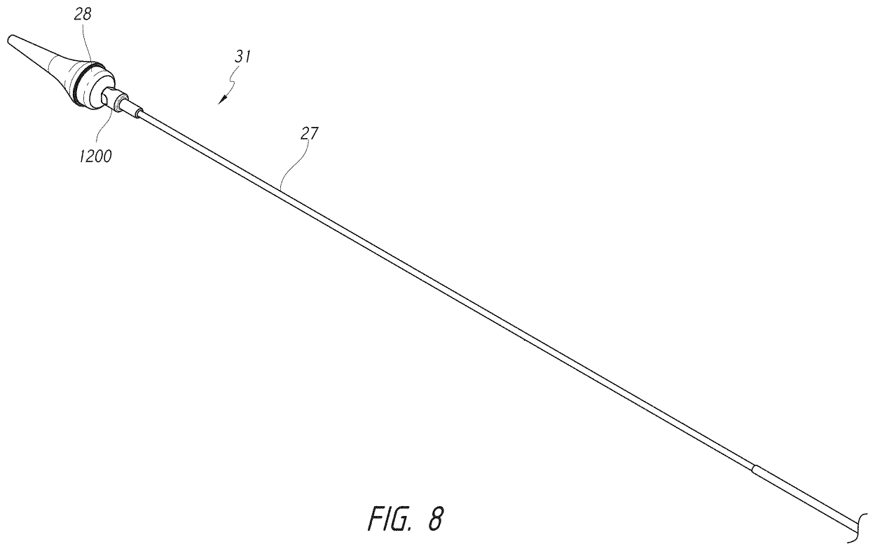

Moving further inwardly from the inner shaft assembly 18 is the nose cone assembly 31 also seen in FIG. 8. This may be a nose cone shaft 27, and in some embodiments, may have a nose cone 28 on its distal end. The nose cone 28 can be made of polyurethane for atraumatic entry and to minimize injury to venous vasculature. The nose cone 28 can also be radiopaque to provide for visibility under fluoroscopy.

The nose cone shaft 27 may include a lumen sized and configured to slidably accommodate a guide wire so that the delivery system 10 can be advanced over the guide wire through the vasculature. However, embodiments of the system 10 discussed herein may not use a guide wire and thus the nose cone shaft 27 can be solid. The nose cone shaft 27 may be connected from the nose cone 28 to the handle, or may be formed of different segments such as the other assemblies. Further, the nose cone shaft 27 can be formed of different materials, such as plastic or metal, similar to those described in detail above.

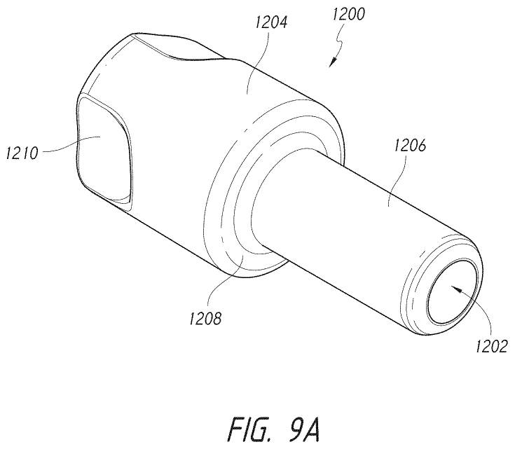

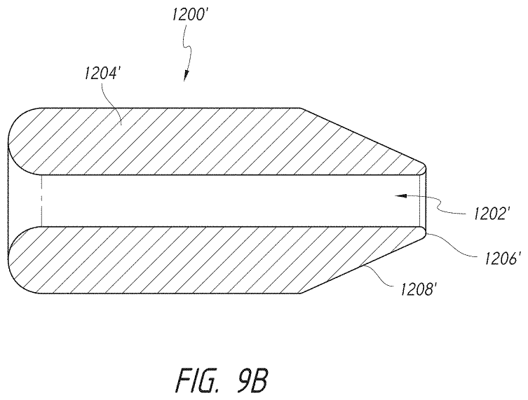

In some embodiments, the nose cone shaft 27 includes a guide wire shield 1200 located on a portion of the nose cone shaft 27. Examples of such a guide wire shield can be found in FIGS. 9A-B. In some embodiments, the guide wire shield 1200 can be proximal to the nose cone 28. In some embodiments, the guide wire shield 1200 can be translatable along the nose cone shaft 27. In some embodiments, the guide wire shield 1200 can be locked in place along the nose cone shaft 27. In some embodiments, the guide wire shield 1200 can be at least partially located within the nose cone 28.

Advantageously, the guide wire shield 1200 can allow for smooth tracking of the guide wire with the implant 70 loaded, and can provide a large axial diameter landing zone for a distal end of the implant so that the distal end of the implant 70 may spread out properly and be arranged in a uniform radial arrangement. This uniformity allows for proper expansion. Furthermore, the guide wire shield 1200 can prevent kinking or damaging of the nose cone shaft 27 during compression/crimping of the prosthesis 70, which can exert a large compressive force on the nose cone shaft 27. As the prosthesis 70 can be crimped onto the guide wire shield 1200 instead of directly on the nose cone shaft 27, the guide wire shield 1200 can provide a protective surface.

As shown, the guide wire shield 1200 can include a lumen 1202 configured to surround the nose cone shaft 27. The guide wire shield 1200 can include a larger diameter distal end 1204 and a smaller diameter proximal end 1206. In some embodiments, the dimension change between the two ends can be tapered, or can be a step 1208 such as shown in FIG. 9A. The distal end 1204 can include a number of indents 1210 for easier gripping by a user, but may not be included in all embodiments. The proximal end 1206 and the distal end 1204 can both be generally cylindrical, but the particular shape of the guide wire shield 1200 is not limiting.

The distal end of the prosthesis 70 can be crimped so that it is radially in contact with the proximal end 1206 of the guide wire shield 1200. This can allow the prosthesis 70 to be properly spread out around an outer circumference of the proximal end 1206 of the guide wire shield 1200. In some embodiments, the distal end of the prosthesis 70 can longitudinally abut against the proximal end of the distal end 1204 (e.g., at the step 1208), thus providing a longitudinal stop.

FIG. 9B shows an alternate embodiment of a guide wire shield 1200′ having a more tapered configuration. As shown, the proximal end 1206′ of the guide wire shield 1200′ can be a single radially outward taper 1208′ to the distal end 1204′ of the guide wire shield 1200′, which can be generally cylindrical. The guide wire shield 1200′ can also include a lumen 1202′ for receiving the nose cone shaft 27.

The nose cone assembly 31 is disposed so as to be individually slidable with respect to the other assemblies. Further, the nose cone assembly 31 can slide distally and proximally relative to the rail assembly 22 together with the outer sheath assembly 22, mid shaft assembly 21, and inner assembly 18.

In some embodiments, one or more spacer sleeves (not shown) can be used between different assemblies of the delivery system 10. For example, a spacer sleeve can be located concentrically between the mid shaft assembly and the rail assembly 20, generally between the mid hypotube 43 and rail hypotube 136. In some embodiments, the spacer sleeve can be generally embedded in the hypotube 43 of the mid shaft assembly 21, such as on an inner surface of the mid shaft assembly 21. In some embodiments, a spacer sleeve can be located concentrically between the rail assembly 20 and the inner assembly 18, generally within the rail hypotube 136. In some embodiments, a spacer sleeve can be used between the outer sheath assembly 22 and the mid shaft assembly 21. In some embodiments, a spacer sleeve can be used between the inner assembly 18 and the nose cone assembly 31. In some embodiments, 4, 3, 2, or 1 of the above-mentioned spacer sleeves can be used. The spacer sleeves can be used in any of the above positions.

The spacer sleeve can be made of a polymer material such as braided Pebax® and can be lined, for example with PTFE, on the inner diameter, though the particular material is not limiting. The spacer sleeve can advantageously reduce friction between the steerable rail assembly 20 and its surrounding assemblies. Thus, the spacer sleeves can act as a buffer between the rail assembly 20 and the inner/nose cone assembly 18/30. Further, the spacer sleeve can take up any gap in radius between the assemblies, preventing compressing or snaking of the assemblies during steering. In some embodiments, the spacer sleeve may include cuts or slots to facilitate bending of the spacer sleeve. In some embodiments, the spacer sleeve may not include any slots, and may be a smooth cylindrical feature.

The spacer sleeve can be mechanically contained by the other lumens and components, and is thus not physically attached to any of the other components, allowing the spacer sleeve to be “floating” in that area. The floating aspect of the spacer sleeve allows it to move where needed during deflection and provide a support and/or lubricious bear surface/surfaces. Accordingly, the floating aspect allows the delivery system 10 to maintain flex forces. However, in some embodiments, the spacer sleeve can be connected to other components.

### Hypotube/Shaft Construction

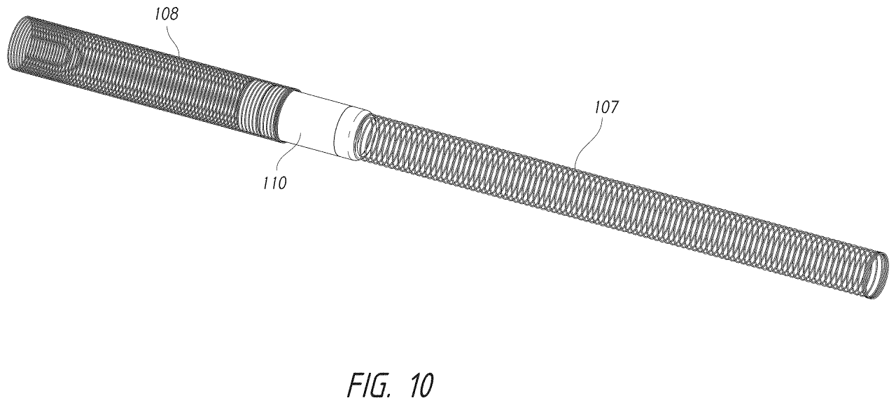

As discussed above, the outer sheath assembly 22, the mid shaft assembly 21, the inner assembly 18, and the rail assembly 20 can contain an outer hypotube 104, a mid shaft hypotube 43, a distal section 126, and a rail hypotube 136, respectively. Each of these hypotubes/sections/shafts can be laser cut to include a number of slots, thereby creating a bending pathway for the delivery system to follow. While different slot assemblies are discussed below, it will be understood that any of the hypotubes can have any of the slot configurations discussed below. FIGS. 10-14 show the different hypotubes in isolated format.

The outer hypotube 104, shown in FIG. 10, can be generally formed of a metal coil or a plurality of metal coils. In some embodiments, the outer hypotube 104 can be formed of a proximal metal coil 107 and a distal metal coil 108. The proximal metal coil 107 and the distal metal coil 108 can be longitudinally separated by a tube portion 110, such as shown in FIG. 10. However, in some embodiments the proximal metal coil 107 and the distal metal coil 108 connect. The proximal metal coil 107 and the distal metal coil 108 can be connected to an outer surface of the tube portion 110, for example at the distal end of the proximal metal coil 107 and a proximal end of the distal metal coil 108, in order to form the full outer hypotube 104. In some embodiments, the proximal metal coil 107 and the distal metal coil 108 are generally the same. In some embodiments, the proximal metal coil 107 and the distal metal coil 110 are different, for example in spacing between coils, curvature, diameter, etc. In some embodiments, the distal metal coil 108 has a larger diameter than the proximal metal coil 107, such as when the distal metal coil 108 forms the large diameter of the capsule 106. In some embodiments, they have the same diameter. In some embodiments, one or both of the metal coils 108/107 can form the capsule 106. The coils can be coated by polymer layers, such as described in detail below regarding the capsule construction. The coil construction can allow the outer hypotube 104 to follow the rail in any desired direction.

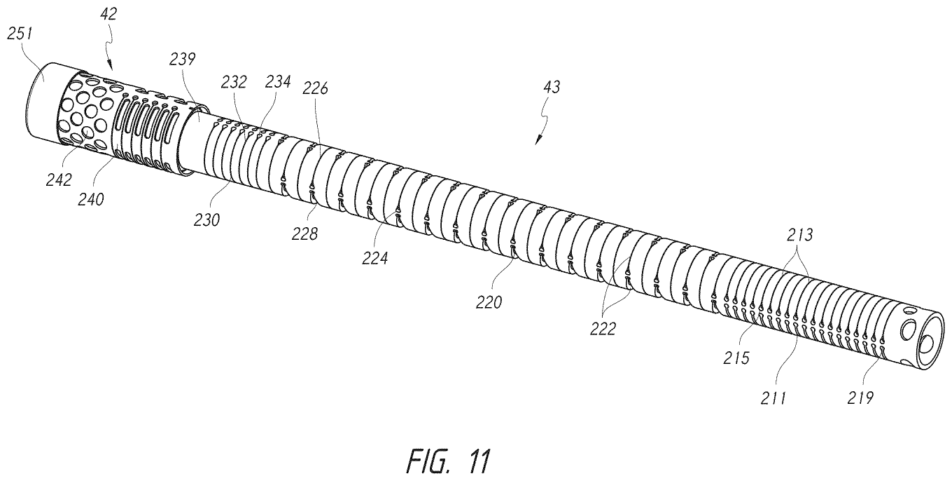

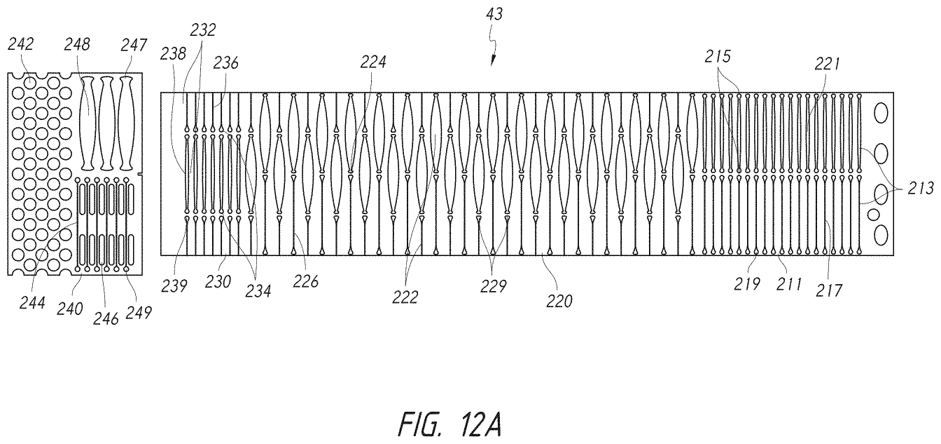

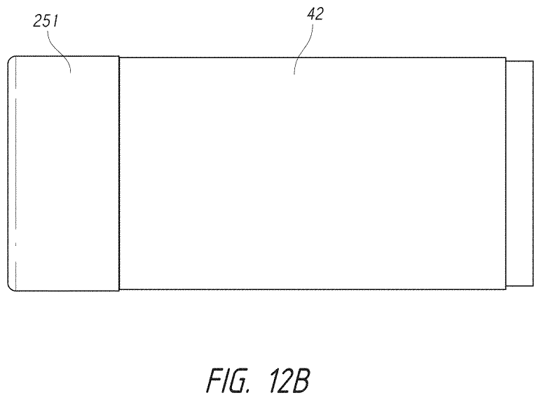

Moving radially inwardly, FIGS. 11-12B shows that the mid shaft hypotube 43 can be a metal laser cut hypotube, such as a lasercut Nitinol hypotube. FIG. 12A illustrates a flat pattern of FIG. 11. As shown in the figures, the hypotube 43 can have a number of slots/apertures cut into the hypotube. In some embodiments, the cut pattern can be the same throughout. In some embodiments, the mid shaft hypotube 43 can have different sections having different cut patterns.

For example, the proximal end of the mid shaft hypotube 43 can be a first section 210 having a plurality circumferentially extending slot pairs 213 spaced longitudinally along the first section 211. Generally, two slots are cut around each circumferential location forming almost half of the circumference. Accordingly, two backbones 215 are formed between the slots 213 extending up the length of the first section 211. The slot pairs 213 can be composed of a first thin slot 217. A second slot 221 of each of the slot pairs 213 can be thicker than the first slot 217, such as 1, 2, 3, 4, or 5 times thicker. In some embodiments, the second slot 217 can be generally the same longitudinal thickness throughout the slot. Each of the slots of the slot pair 213 can end in a teardrop shape 219 in some embodiments to facilitate bending.

Moving distally, the mid shaft hypotube 43 can include a second section 220 having a number of slot pairs 222. Similar to the first section 211, the second section 220 can have a plurality of circumferentially extending slots spaced longitudinally along the second section 220. Generally, two slots (e.g., one slot pair 222) are cut around each circumferential location, forming almost half of a circumference. Accordingly, “backbones” 224 can be formed between the slots extending up the length of the second section 220. Each slot pair 222 can include a first slot 226 that is generally thin and has no particular shape (e.g., it can look the same as the slots 213 in the first section 211), and a second slot 228 that is significantly longitudinally thicker than the first slot 226. The second slot 228 can be narrower at its ends and longitudinally thicker in its middle portion, thereby forming a curved slot. Moving longitudinally along the second section 220, each slot pair 222 can be offset approximately 45 or 90 degrees as compared to longitudinally adjacent slot pairs 222. In some embodiments, a second slot pair 222 is offset 90 degrees from an adjacent first slot pair 222, and a third slot pair 222 adjacent the second slot pair 222 can have the same configuration of the first slot pair 222. This repeating pattern can extend along a length of the second section 220, thereby providing a particular bending direction induced by the second slot 228 of the slot pairs 222. Accordingly, the “backbone” 224 shifts circumferential position due to the offsetting of adjacent shifting slot pairs 222. Each of the slots of the slot pair 222 can end in a teardrop shape 229 in some embodiments to facilitate bending.

Moving distally, the mid shaft hypotube 43 can have a third section 230 having a number of slots. The outer retention ring 240 can be attached to a distal end of the third section 230. The third section 230 can have circumferentially extending slot pairs 232, each slot on the slot pair extending about half way along the circumference to form the two backbones 234. The slot pairs 232 can be composed of a first thin slot 236, similar to the slots 213 discussed in the first section 211. A second slot 238 of each of the slot pairs 232 can be thicker than the first slot 236, such as 1, 2, 3, 4, or 5 times thicker. In some embodiments, the second slot 238 can be generally the same longitudinal thickness throughout the slot, unlike the second slot 228 of the second section 220. The first slots 236 and the second slots 238 can be circumferentially aligned along a length of the third section 230 so that all of the first slots 236 are in the same circumferential position and all of the second slots 238 are in the same circumferential position. The second slots 238 can be aligned with one of the circumferential positions of the second slots 228 of the second section 220. Each of the slots of the slot pair 232 can end in a teardrop shape 239 in some embodiments to facilitate bending.

In some embodiments, an outer retention ring strengthener 240 which can partially or fully circumferentially surround the outer retention member 40 can have a number of slots/holes/apertures as well, such as shown in FIGS. 11-12. This can allow it to bend over curves, especially tight curves. In some embodiments, the distal end of the strengthener 240 includes a number of generally circular/elliptical holes 242. This can last for approximately half of the length of the strengthener 240. On the proximal half, one circumferential half of the strengthener 240 can include repeating thin slots 244 spaced by elongate ovoid holes 246. For example, two circumferentially spaced apart elongate ovoid holes 246 can be between each thin slot 244. Each of the slots 244 can end in a teardrop shape 249 in some embodiments to facilitate bending. On the other circumferential half of the proximal section, the strengthener 240 can include a number of large slots 248, for example 1, 2, 3, 4, or 5 large slots 248 spaced longitudinally apart. The large slots 248 can be larger in the middle and narrow towards each circumferential end. The large slots 248 may include ending expansions 247 to facilitate flexibility.

Additionally, the outer retention strengthener 240 can provide strength to lower deployment forces, protect the prosthesis 70 from any metal layers, and can add strength. In some embodiments, the liner can 240 be a polymer, such as PTFE, though the type of polymer or material is not limiting. In some embodiments, the strengthener 240 can be a metal. In some embodiments, the strengthener 240 can further include an outer polymer layer/jacket, such as a Pebax® jacket. This prevents the strengthener 240 from catching on the outer sheath assembly 22.

In certain embodiments, the outer retention ring 42 can further include an inner liner for smoothly transitioning over the prosthesis 70. The inner liner can be PTFE or etched PTFE, though the particular material is not limiting and other reduced friction polymers can be used. As shown in FIG. 12B, to prevent delamination during loading of the implant 70, the liner 251 may not be flush at the distal end of the outer retention ring 42. Instead, the liner 251 can be extended and inverted at the distal end in order to cover the distal end of the outer retention ring 42. In some embodiments, the liner 251 can cover an outer surface of the strengthener 240 as well. This can create a seamless rolled reinforced tip of the liner 251. The liner 251 can fully or partially cover an outer surface of the outer retention ring 42, for example ¼, ⅓, ½, ⅔, ¾ (or greater than ¼, ⅓, ½, ¾), or all of the outer retention ring 42. This solution is advantageous over previously known methods, such as disclosed in U.S. Pat. No. 6,622,367, incorporated by reference in its entirety, as PTFE lined applications do not adhere particularly well to reinforcements or the outer jacket. By inverting the liner 251 and fusing it to the outer retention ring 42 and/or the strengthener 240 and/or an outer polymer jacket on the strengthener 240/outer retention ring 42, this creates a seamless reinforced tip that can mitigate delamination. Delamination is a serious concern because the delaminated liner can tear and embolize during deployment, and the delaminated layer can cause extremely high loading and deployment forces. Delaminated layers can also cause lumen translation problems by locking up shafts thereby adding translational force requirements.

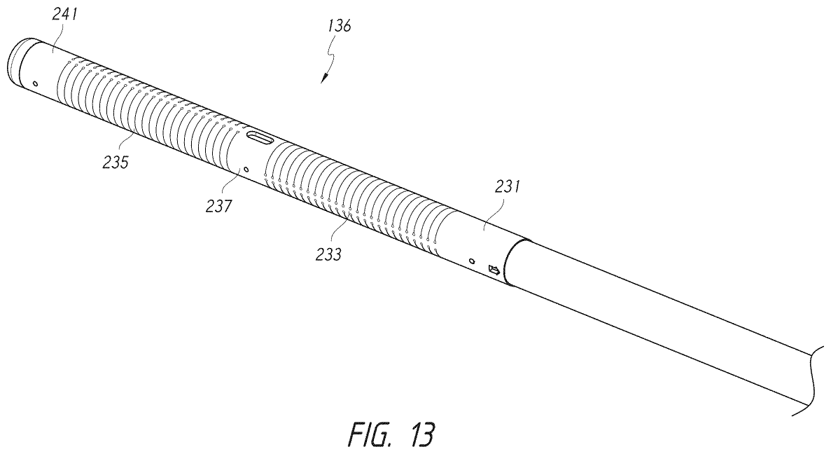

Next, again moving radially inward, FIG. 13 shows an embodiment of the rail hypotube 136 (distal end towards the right). The rail hypotube 136 can also contain a number of circumferential slots. The rail hypotube 136 can generally be broken into a number of different sections. At the most proximal end is an uncut (or unslotted) hypotube section 231. Moving distally, the next section is the proximal slotted hypotube section 133. This section includes a number of circumferential slots cut into the rail hypotube 136. Generally, two slots are cut around each circumferential location forming almost half of the circumference. Accordingly, two backbones are formed between the slots extending up the length of the hypotube 136. This is the section that can be guided by the proximal pull wires 140. Moving further distally is the location 237 where the proximal pull wires 140 connect, and thus slots can be avoided. Thus section is just distal of the proximally slotted section.

Distally following the proximal pull wire connection area is the distal slotted hypotube section 235. This section is similar to the proximal slotted hypotube section 233, but has significantly more slots cut out in an equivalent length. Thus, the distally slotted hypotube section 235 provides easier bending than the proximally slotted hypotube section 233. In some embodiments, the proximal slotted section 233 can be configured to experience a bend of approximately 90 degrees with a half inch radius whereas the distal slotted section 135 can bend at approximately 180 degrees within a half inch. Further, as shown in FIG. 13, the spines of the distally slotted hypotube section 235 are offset from the spines of the proximally slotted hypotube section 233. Accordingly, the two sections will achieve different bend patterns, allowing for three-dimensional steering of the rail assembly 20. In some embodiments, the spines can be offset 30, 45, or 90 degrees, though the particular offset is not limiting. In some embodiments, the proximally slotted hypotube section 233 can include compression coils. This allows for the proximally slotted hypotube section 233 to retain rigidity for specific bending of the distally slotted hypotube section 235.

At the distalmost end of the distal slotted hypotube section 235 is the distal pull wire connection area 241 which is again a non-slotted section of the rail hypotube 136.

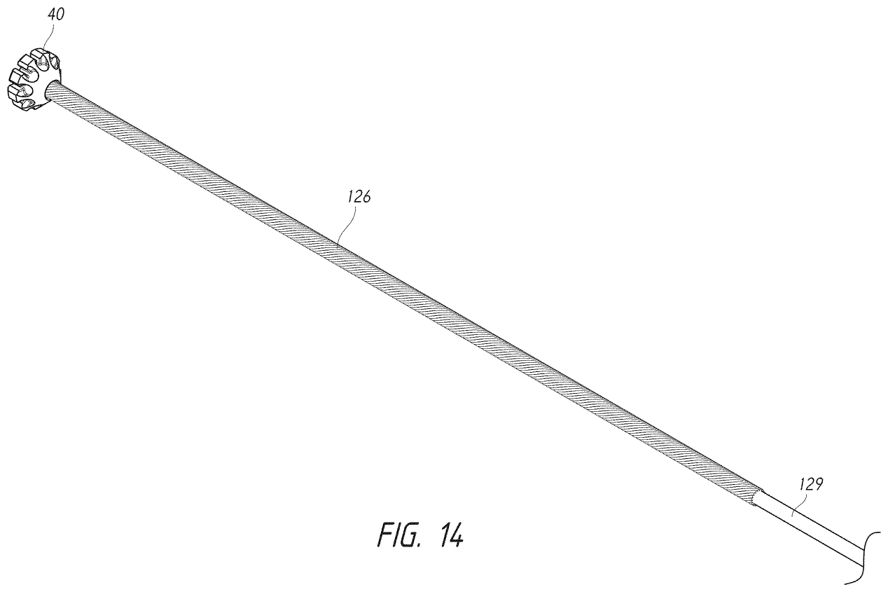

Moving radially inwardly in FIG. 14, the inner assembly 18 is composed generally of two sections. The proximal section is a hypotube 129, either slotted or non-slotted. The distal section 126, which at least partially overlaps an outer surface of the proximal hypotube 129, can be designed to be particularly flexible. For example, the distal section 126 can be more flexible than any of the other shafts discussed herein. In some embodiments, the distal section 126 can be more flexible than any shaft discussed herein other than the nose cone shaft 27. In some embodiments, the distal section 126 can be a flexible tube or hypotube. In some embodiments, the distal section 126 can be a cable, such as a flexible cable. For example, the cable can several strands of wire, such as metal, plastic, polymer, ceramic, etc., wound together to form a rope or cable. Because the cable is so flexible, it can more easily bend with the rail assembly 20. Further, the cable can be smooth, which allows the rail assembly 20 to track over a smooth surface, eliminating the need for any inner liner on the rail assembly 20.

### Capsule Construction

The capsule 106 can be formed from one or more materials, such as PTFE, ePTFE, polyether block amide (Pebax®), polyetherimide (Ultem®), PEEK, urethane, Nitinol, stainless steel, and/or any other biocompatible material. The capsule 106 is preferably compliant and flexible while still maintaining a sufficient degree of radial strength to maintain a replacement valve 70 within the capsule 106 without substantial radial deformation, which could increase friction between the capsule 106 and a replacement valve 70 contained therein. The capsule 106 also preferably has sufficient column strength to resist buckling of the capsule 106, and sufficient tear resistance to reduce or eliminate the possibility of the replacement valve 70 tearing and/or damaging the capsule 106. Flexibility of the capsule 106 can be advantageous, particularly for a transseptal approach. For example, while being retracted along a curved member, for example while tracking over a rail assembly as described herein, the capsule 106 can flex to follow the curved member without applying significant forces upon the curved member, which may cause the curved member to decrease in radius. More specifically, the capsule 106 can bend and/or kink as it is being retracted along such a curved member such that the radius of the curved member is substantially unaffected.

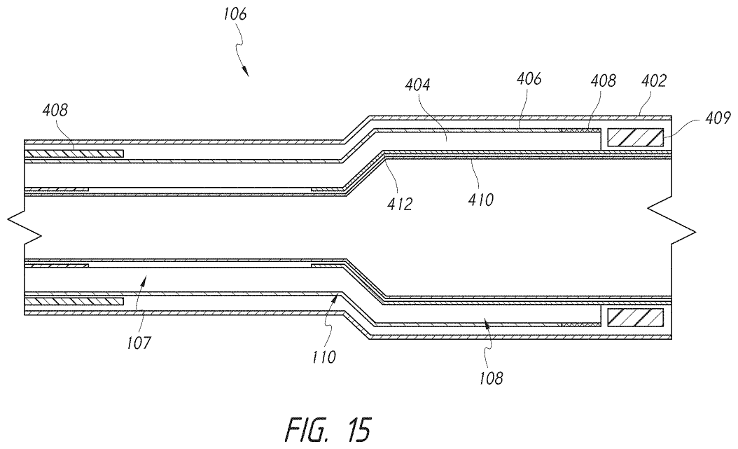

FIG. 15 shows embodiments of a capsule 106 that can be used with embodiments of the delivery system 10. The capsule 106 may include any of the materials and properties discussed above. With many implant capsules, compression resistance and flexibility are typically balanced together, as improved flexibility can lead to worse compression resistance. Thus, there tends to be a choice made between compression resistance and flexibility. However, disclosed are embodiments of a capsule 106 that can achieve both high compression resistance as well as high flexibility. Specifically, the capsule 106 can bend in multiple directions.

In particular, a metal hypotube can provide radial strength and compression resistance, while specific slots/cuts in the hypotube can enable the flexibility of the capsule 106. In some embodiments, a thin liner and a jacket can surround the capsule 106, such as a polymer layer, to prevent any negative interactions between the implant 70 and the capsule 106.

In some embodiments, the capsule 106 can have a particular construction to allow for it to achieve advantageous properties, as shown in FIG. 15. The capsule 106 can be made of several different layers to provide such properties.

In some embodiments, the capsule 106 can be formed of a metal layer 404, which gives the capsule 106 its structure. This metal layer 404 can include the coils discussed with respect to FIG. 10, or could be one or more hypotubes. The capsule 106 is then covered on an outer surface by a polymer layer and on an inner surface by a liner. All of these features are discussed in detail below.

As mentioned, the metal layer 404 can be, for example, a metal hypotube or laser cut hypotube. In some embodiments, the metal layer 404 can be a metal coil or helix, as discussed in detail above with respect to FIG. 10. Though not limiting, the metal layer 404 can have a thickness of 0.007 inches (or about 0.007 inches).

If a metal coil, such as shown in FIG. 10, is used, the coil dimensions can stay the same throughout a length of the metal layer 404. However, in some embodiments the coil dimensions can vary along a length of the metal layer 404. For example, the coils can vary between coils having a 0.014-inch gap with a 0.021-inch pitch (e.g., small coils), coils having a 0.020 inch-gap with a 0.02-inch pitch (e.g., large coils), and coils having a 0.020-inch gap with a 0.027-inch pitch (e.g., spaced large coils). However, these particular dimensions are merely examples, and other designs can be used as well.

The distalmost end of the metal layer 404 can be formed out of the small coils. Moving proximally, the metal layer 404 may then transition to a section of large coils, followed again by a section of small coils, and then finally the proximalmost section can be the spaced large coils. As an example set of lengths, though not limiting, the distalmost small coil section may have a length of 10 mm (or about 10 mm). Moving proximally, the adjacent large coil section may extend 40 mm (or about 40 mm) to 60 mm (or about 60 mm) in length. These two sections would be found in the distal metal coil 108 shown in FIG. 10. Moving to the proximal metal coil 107 shown in FIG. 10, the small coil section can have a length of 10 mm (or about 10 mm). The remaining portion of the proximal metal coil 107 can be the spaced large coil section. The spaced large coil section can have a length of 40 mm (or about 40 mm) to 60 mm (or about 60 mm) or greater.

As mentioned, the metal layer 404 (either coil or hypotube) can be covered by an outer polymer layer or jacket 402. In some embodiments, the outer polymer 402 layer is an elastomer, though the particular material is not limiting. In some embodiments, the outer polymer layer 402 can comprise polytetrafluoroethylene (PTFE) or expanded polytetrafluoroethylene (ePTFE). The ePTFE can have very different mechanical properties that PTFE. For example, ePTFE can be much more flexible while still maintaining good tensile/elongation properties. In some embodiments, the outer polymer layer 402 can comprise a thermoplastic elastomer, such as PEBAX®. In some embodiments, the outer polymer layer 402 can be pre-axially stressed before applying to the capsule. The outer polymer layer 402 can be approximately 0.006 to 0.008 inches in thickness, but the particular thickness is not limiting.

The outer polymer layer 402 can be applied to the metal layer 404 to form an outer jacket, such as by reflowing the polymer. In some embodiments, the outer polymer layer 402 can be directly applied to the metal layer 404. In some embodiments, an adhesive layer 406 can be disposed between the metal layer 404 and the outer polymer layer 402 to promote attachment of the outer polymer layer to the metal layer. For example, a fluoropolymer, or other soft durometer fluoroelastomer, can be applied between the metal layer 404 and the outer layer 402 in order to attach the two layers together and prevent delamination. In some embodiments, the adhesive layer 406 is not used.

In some implementations, other materials can be included between the metal layer 404 and the outer polymer layer 402 in order to improve properties. For example, fluorinated ethylene propylene (FEP) sections 408 can improve radial strength, in particular when the implant is under compression. While an FEP layer 408 is discussed as a particular material, other high strength polymers, metals, or ceramics can be used as well, and the particular material is not limiting. The FEP layer 408 can also act as an adhesive in some instances.

FEP sections 408 can be included at the distal and proximal ends of the capsule 106. The FEP sections 408 can either overlap the adhesive layer 406. Thus, FEP sections 408 can be located between the adhesive layer 406 and the metal layer 404 or between the adhesive layer 406 and the outer polymer layer 402. In some embodiments, the FEP sections 408 may be located in sections of the capsule 106 that do not include an adhesive layer 406.

The FEP section 408 located at the distal end of the capsule 106 can have a length of 10 mm (or about 10 mm), thought he particular length is not limiting. In some embodiments, the FEP section 408 is approximately 0.003 inches in thickness, but the thickness may vary and is not limited by this disclosure. In some embodiments, different FEP sections 408 (e.g., a proximal section and a distal section) can have different thicknesses. In some embodiments, all FEP 408 layers have the same thickness. Example thicknesses can be 0.006 inches or 0.003 inches.

Moving to the inside of the metal layer 404, a liner 410 can be included on its radially inner surface. The liner 410 can be formed of a low friction and/or high lubricity material that allows for the capsule 106 to be translated over the prosthesis 70 without catching or damaging portions of the prosthesis 70. In some embodiments, the liner 410 can be PTFE, which can resist radial expansion and decrease friction with the prosthesis 70.

In some embodiments, the liner 410 is made from ePTFE. However, it can be difficult to reflow a standard ePTFE liner 410 on the inner layer of the capsule 106. Accordingly, the ePTFE liner layer 410 can be pre-compressed before applying onto the inner layer of the capsule 106. In some embodiments, portions of the outer polymer layer 402 and the liner 410 can be in contact with one another. Thus, prior to bonding the two layers together, the ePTFE liner 410 and/or outer polymer layer 402 can be axially compressed. Then, the layers can be bonded together with reflow techniques during manufacturing. For example, the ePTFE liner 410 can be axially compressed, such as over a mandrel, while the outer polymer layer 402 can be placed over it. These two layers can then be reflowed (e.g., melting under pressure) to connect. The combined layers can be slid into and/or around the metal layer 404 discussed herein, and can be melted under pressure again to form the final capsule 106. This technique can allow for the capsule 106 to maintain flexibility and prevent breakage/tearing.

As mentioned above, the inner liner 410 can be ePTFE in some embodiments. The surface friction of ePTFE can be about 15% less than standard PTFE, and can be about 40% less than standard extruded thermoplastics that are used in the art.

In certain embodiments, the liner layer 410 can extend only along an inner surface of the capsule 106 and terminate at a distal end. However, to prevent delamination during loading of the implant 70, the liner 410 may not be flush at the distal end of the capsule 106. Instead, the liner 410 can be extended and inverted at the distal end in order to cover the distal end of the capsule 106 as well as an outer diameter of a portion of the outer polymer layer 402. This can create a seamless rolled reinforced tip of the liner 410. This solution is advantageous over previously known methods, such as disclosed in U.S. Pat. No. 6,622,367, incorporated by reference in its entirety, as PTFE lined applications do not adhere particularly well to reinforcements or the outer jacket. By inverting the liner 410 and fusing it with the outer polymer layer 402, this creates a seamless reinforced capsule tip that can mitigate delamination. Delamination is a serious concern because the delaminated liner can tear and embolize during deployment, and the delaminated layer can cause extremely high loading and deployment forces. Delaminated layers can also cause lumen translation problems by locking up shafts thereby adding translational force requirements.

In some embodiments, another FEP section 412 can be included between the liner 410 and the metal layer 404. The FEP section 412 can be located on distal metal coil 108, as well as the tube 110 transitioning between the distal metal coil 108 and the proximal metal coil 107. In some embodiments, the FEP section 412 may continue partially or fully into the proximal metal coil 107.

In some embodiments, an FEP section 412 can be included in the proximalmost portion of the proximal metal coil 107. This FEP section 412 be approximately 0.5 inches in length. In some embodiments, there is a longitudinal gap between the proximalmost FEP section 412 and the FEP section 412 that extends over the distal metal coil 108. In some embodiments, the previously mentioned FEP sections 412 are continuous.

As shown in FIG. 15, the metal layer 404 may stop proximal to the edges of the outer polymer layer 402, liner 410, and FEP section 412. If so, a thicker portion of an adhesive layer 409 can be applied at the distal end of the metal layer 404 to match the distal end of the other layers. However, this section can be removed during manufacture, so the distal end of the metal layer 404 is the distal end of the capsule 106, which can then be covered by the liner 410. In some embodiments, the extended sections distal to the metal layer 404 are not used.

### Handle

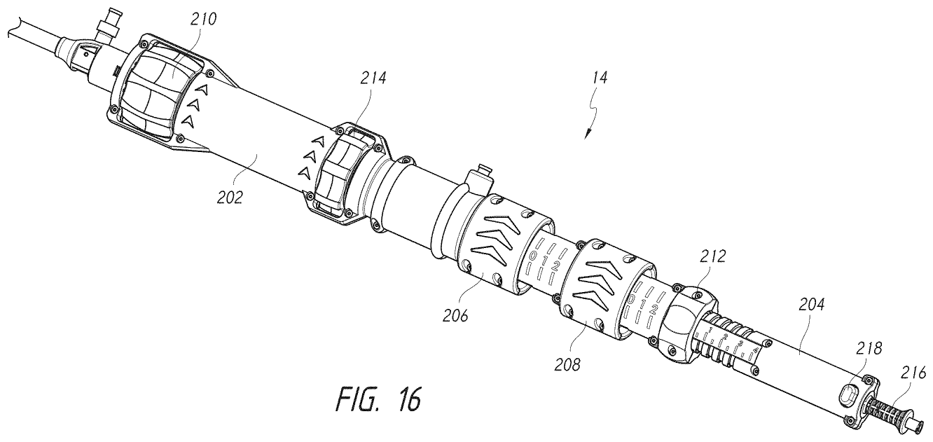

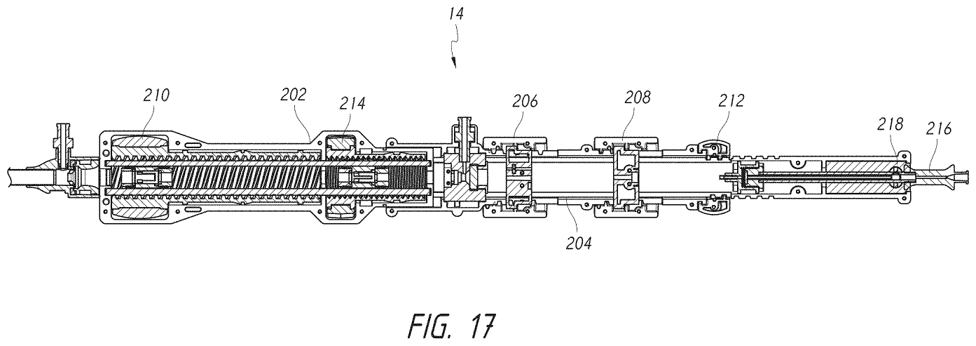

The handle 14 is located at the proximal end of the delivery system 10 and is shown in FIG. 16. A cross-section of the handle 14 is shown in FIG. 17. The handle 14 can include a number of actuators, such as rotatable knobs, that can manipulate different components of the delivery system 10. The operation of the handle 10 is described with reference to delivery of a replacement mitral valve prosthesis 70, though the handle 10 and delivery system 10 can be used to deliver other devices as well.

The handle 14 is generally composed of two housings, a rail housing 202 and a delivery housing 204, the rail housing 202 being circumferentially disposed around the delivery housing 204. The inner surface of the rail housing 202 can include a screwable section configured to mate with an outer surface of the delivery housing 204. Thus, the delivery housing 204 is configured to slide (e.g., screw) within the rail housing 202, as detailed below. The rail housing 202 generally surrounds about one half the length of the delivery housing 204, and thus the delivery housing 204 extends both proximally and distally outside of the rail housing 202.

The rail housing 202 can contain two rotatable knobs, a distal pull wire knob 206 and a proximal pull wire knob 208. However, the number of rotatable knobs on the rail housing 202 can vary depending on the number of pull wires used. Rotation of the distal pull wire knob 206 can provide a proximal force, thereby providing axial tension on the distal pull wires 138 and causing the distal slotted section 135 of the rail hypotube 136 to bend. The distal pull wire knob 206 can be rotated in either direction, allowing for bending in either direction, which can control anterior-posterior angles. Rotation of the proximal pull wire knob 208 can provide a proximal force, and thus axial tension, on the proximal pull wires 140, thereby causing the proximal slotted section 133 of the rail hypotube 136 to bend, which can control the medial-lateral angle. The proximal pull wire knob 108 can be rotated in either direction, allowing for bending in either direction. Thus, when both knobs are actuated, there can be two bends in the rail hypotube 136, thereby allowing for three-dimensional steering of the rail shaft 132, and thus the distal end of the delivery system 10. Further, the proximal end of the rail shaft 132 is connected on an internal surface of the rail housing 202.

The bending of the rail shaft 132 can be used to position the system, in particular the distal end, at the desired patient location, such as at the native mitral valve. In some embodiments, rotation of the pull wire knobs 206/208 can help steer the distal end of the delivery system 10 through the septum and left atrium and into the left ventricle so that the prosthesis 70 is located at the native mitral valve.

Moving to the delivery housing 204, the proximal ends of the inner shaft assembly 19, outer sheath assembly 22, mid shaft assembly 21, and nose cone shaft assembly 30 can be connected to an inner surface of the delivery housing 204 of the handle 14. Thus, they can move axially relative to the rail assembly 20 and rail housing 202.

A rotatable outer sheath knob 210 can be located on the distal end of the delivery housing 204, being distal to the rail housing 202. Rotation of the outer sheath knob 210 will pull the outer sheath assembly 22 in an axial direction proximally, thus pulling the capsule 106 away from the implant 70 and releasing the distal end 301 of implant 70. Thus the outer sheath assembly 22 is individually translated with respect to the other shafts in the delivery system 10. The distal end 303 of the implant 70 can be released first, while the proximal end 301 of the implant 70 can remain radially compressed between the inner retention member 40 and the outer retention member 42.

A rotatable mid shaft knob 214 can be located on the delivery housing 204, in some embodiments proximal to the rotatable outer sheath knob 210, being distal to the rail housing 202. Rotation of the mid shaft knob 214 will pull the mid shaft assembly 21 in an axial direction proximally, thus pulling the outer retention ring 42 away from the implant 70 and uncovering the inner retention member 40 and the proximal end 301 of the implant 70, thereby releasing the implant 70. Thus, the mid shaft assembly 21 is individually translated with respect to the other shafts in the delivery system 10.

Located on the proximal end of the delivery housing 204, and thus proximal to the rail housing 202, can be a rotatable depth knob 212. As the depth knob 212 is rotated, the entirety of the delivery housing 204 moves distally or proximally with respect to the rail housing 202 which will remain in the same location. Thus, at the distal end of the delivery system 10, the inner shaft assembly 18, outer sheath assembly 22, mid shaft assembly 21, and nose cone shaft assembly 31 together (e.g., simultaneously) move proximally or distally with respect to the rail assembly 20 while the implant 70 remains in the compressed configuration. In some embodiments, actuation of the depth knob 212 can sequentially move the inner shaft assembly 18, outer sheath assembly 22, mid shaft assembly 21, and nose cone shaft assembly 31 relative to the rail assembly 20. In some embodiments, actuation of the depth knob 212 can together move the inner shaft assembly 18, outer sheath assembly 22, and mid shaft assembly 21 relative to the rail assembly 20. Accordingly, the rail shaft 132 can be aligned at a particular direction, and the other assemblies can move distally or proximally with respect to the rail shaft 132 for final positioning while not releasing the implant 70. The components can be advanced approximately 1, 2, 3, 5, 6, 7, 8, 9, or 10 cm along the rail shaft 132. The components can be advanced more than approximately 1, 2, 3, 5, 6, 7, 8, 9, or 10 cm along the rail shaft 132. An example of this is shown in FIG. 2C. The capsule 106 and outer retention ring 42 can then be individually withdrawn with respect to the inner assembly 18 as discussed above, in some embodiments sequentially, releasing the implant 70. The assemblies other than the rail assembly 20 can then be withdrawn back over the rail shaft 132 by rotating the depth knob 212 in the opposite direction.

The handle 14 can further include a mechanism (knob, button, handle) 216 for moving the nose cone shaft 27, and thus the nose cone 28. For example, a knob 216 can be a portion of the nose cone assembly 31 that extends from a proximal end of the handle 14. Thus, a user can pull or push on the knob 216 to translate the nose cone shaft 27 distally or proximally individually with respect to the other shafts. This can be advantageous for proximally translating the nose cone 28 into the outer sheath assembly 22/capsule 106, thus facilitating withdraw of the delivery system 10 from the patient.

In some embodiments, the handle 14 can provide a lock 218, such as a spring lock, for preventing translation of the nose cone shaft 27 by the knob 216 discussed above. In some embodiments, the lock 218 can be always active, and thus the nose cone shaft 27 will not move without a user disengaging the lock 218. The lock can be, for example, a spring lock that is always engaged until a button 218 on the handle 14 is pressed, thereby releasing the spring lock and allowing the nose cone shaft 27 to translate proximally/distally. In some embodiments, the spring lock 218 allows one-way motion, either proximal or distal motion, of the nose cone shaft 27 but prevents motion in the opposite direction.

The handle 14 can further include a communicative flush port for flushing out different lumens of the delivery system 10. In some embodiments, a single flush port on the handle 14 can provide fluid connection to multiple assemblies. In some embodiments, the flush port can provide fluid connection to the outer sheath assembly 22. In some embodiments, the flush port can provide fluid connection to the outer sheath assembly 22 and the mid shaft assembly 21. In some embodiments, the flush port can provide fluid connection to the outer sheath assembly 22, the mid shaft assembly 21, and the rail assembly 20. In some embodiments, the flush port can provide fluid connection to the outer sheath assembly 22, the mid shaft assembly 21, the rail assembly 20, and the inner assembly 18. Thus, in some embodiments, the rail shaft 132, the outer retention ring 42, and the capsule 406 can all be flushed by a single flush port.

### Valve Delivery Positioning

Methods of using the delivery system 10 in connection with a replacement mitral valve will now be described. In particular, the delivery system 10 can be used in a method for percutaneous delivery of a replacement mitral valve to treat patients with moderate to severe mitral regurgitation. The below methods are merely examples of the how the delivery system may be used. It will be understood that the delivery systems described herein can be used as part of other methods as well.

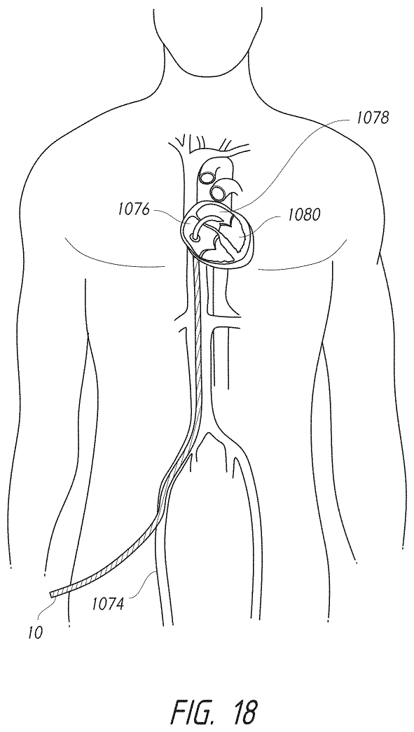

As shown in FIG. 18, in one embodiment the delivery system 10 can be placed in the ipsilateral femoral vein 1074 and advanced toward the right atrium 1076. A transseptal puncture using known techniques can then be performed to obtain access to the left atrium 1078. The delivery system 10 can then be advanced in to the left atrium 1078 and then to the left ventricle 1080. FIG. 18 shows the delivery system 10 extending from the ipsilateral femoral vein 1074 to the left atrium 1078. In embodiments of the disclosure, a guide wire is not necessary to position the delivery system 10 in the proper position, although in other embodiments, one or more guide wires may be used.

Accordingly, it can be advantageous for a user to be able to steer the delivery system 10 through the complex areas of the heart in order to position a replacement mitral valve in line with the native mitral valve. This task can be performed with or without the use of a guide wire with the above disclosed system. The distal end of the delivery system can be advanced into the left atrium 1078. A user can then manipulate the rail assembly 20 to target the distal end of the delivery system 10 to the appropriate area. A user can then continue to pass the bent delivery system 10 through the transseptal puncture and into the left atrium 1078. A user can then further manipulate the delivery system 10 to create an even greater bend in the rail assembly 20. Further, a user can torque the entire delivery system 10 to further manipulate and control the position of the delivery system 10. In the fully bent configuration, a user can then place the replacement mitral valve in the proper location. This can advantageously allow delivery of a replacement valve to an in-situ implantation site, such as a native mitral valve, via a wider variety of approaches, such as a transseptal approach.

The rail assembly 20 can be particularly advantageous for entering into the native mitral valve. As discussed above, the rail assembly 20 can form two bends, both of which can be located in the left atrium 1078. The bends in the rail assembly 20 can position the prosthesis 70, located in the implant retention area 16, so that it is coaxial with the native mitral valve. Once the prosthesis 70 is coaxial, the outer sheath assembly 22, mid shaft assembly 21, inner assembly 18, and nose cone assembly 31 can together be advanced (e.g., using the depth knob 212 of the handle 14) distally relative to the rail assembly 20. These assemblies advance straight off of the rail assembly 20, thus advancing them coaxial with the native mitral valve until the prosthesis 70 is to be released while maintain the prosthesis 70 in the compressed configuration, as discussed below. Thus, the rail assembly 20 provides the ability for a user to lock the angular position in place, so that the user then has to just longitudinally advance the other assemblies over the rail assembly 20 while not needed to make any angular changes, greatly simplifying the procedure. The rail assembly 20 acts as an independent steering assembly, where all the assembly does is provide steerability and no further prosthesis release functionality. Further, the construction of the rail assembly 20 as described above is sufficiently rigid so that when the rail assembly is actuated to its bent shape, movement of the other components, e.g., the outer sheath assembly 22, mid shaft assembly 21, inner assembly 18, and/or nose cone assembly 31, the rail assembly 20 maintains its shape. Thus, the rail assembly 20 can remain in the desired bent position during the sliding of the other assemblies relative to the rail assembly 20, and the rail assembly 20 can help direct the other assemblies to the final position. The proximal/distal translation of the other assemblies over the rail assembly 20 allows for ventricular-atrial motion. In addition, once the distal anchors 80 of the prosthesis 70 have been released in the left ventricle 1080, but prior to full release, the other assemblies can be proximally retracted over the rail assembly 20 to capture any leaflets or chordae.

Reference is now made to FIG. 19 which illustrates a schematic representation of a portion of an embodiment of a replacement heart valve (prosthesis 70) positioned within a native mitral valve of a heart 83. Further details regarding how the prosthesis 70 may be positioned at the native mitral valve are described in U.S. Publication No. 2015/0328000A1, the entirety of which is hereby incorporated by reference, including but not limited to FIGS. 13A-15 and paragraphs [0036]-[0045]. A portion of the native mitral valve is shown schematically and represents typical anatomy, including a left atrium 1078 positioned above an annulus 1106 and a left ventricle 1080 positioned below the annulus 1106. The left atrium 1078 and left ventricle 1080 communicate with one another through a mitral annulus 1106. Also shown schematically in FIG. 19 is a native mitral leaflet 1108 having chordae tendineae 1110 that connect a downstream end of the mitral leaflet 1108 to the papillary muscle of the left ventricle 1080. The portion of the prosthesis 70 disposed upstream of the annulus 1106 (toward the left atrium 1078) can be referred to as being positioned supra-annularly. The portion generally within the annulus 1106 is referred to as positioned intra-annularly. The portion downstream of the annulus 1106 is referred to as being positioned sub-annularly (toward the left ventricle 1080).

As shown in FIG. 19, the replacement heart valve (e.g., prosthesis 70) can be positioned so that the mitral annulus 1106 is located the distal anchors 80 and the proximal anchors 82. In some situations, the prosthesis 70 can be positioned such that ends or tips of the distal anchors 80 contact the annulus 1106 as shown, for example, in FIG. 19. In some situations, the prosthesis 70 can be positioned such that ends or tips of the distal anchors 80 do not contact the annulus 1106. In some situations, the prosthesis 70 can be positioned such that the distal anchors 80 do not extend around the leaflet 1108.

As illustrated in FIG. 19, the replacement heart valve 70 can be positioned so that the ends or tips of the distal anchors 80 are on a ventricular side of the mitral annulus 1106 and the ends or tips of the proximal anchors 82 are on an atrial side of the mitral annulus 1106. The distal anchors 80 can be positioned such that the ends or tips of the distal anchors 80 are on a ventricular side of the native leaflets beyond a location where chordae tendineae 1110 connect to free ends of the native leaflets. The distal anchors 80 may extend between at least some of the chordae tendineae 1110 and, in some situations such as those shown in FIG. 19, can contact or engage a ventricular side of the annulus 1106. It is also contemplated that in some situations, the distal anchors 80 may not contact the annulus 1106, though the distal anchors 80 may still contact the native leaflet 1108. In some situations, the distal anchors 80 can contact tissue of the left ventricle 104 beyond the annulus 1106 and/or a ventricular side of the leaflets.

During delivery, the distal anchors 80 (along with the frame) can be moved toward the ventricular side of the annulus 1106, such as by translating the other assemblies (e.g., outer sheath assembly 22, mid shaft assembly 21, inner assembly 18, and nose cone assembly 31) proximally with respect to the rail assembly 20, with the distal anchors 80 extending between at least some of the chordae tendineae 1110 to provide tension on the chordae tendineae 1110. The degree of tension provided on the chordae tendineae 1110 can differ. For example, little to no tension may be present in the chordae tendineae 1110 where the leaflet 1108 is shorter than or similar in size to the distal anchors 80. A greater degree of tension may be present in the chordae tendineae 1110 where the leaflet 1108 is longer than the distal anchors 80 and, as such, takes on a compacted form and is pulled proximally. An even greater degree of tension may be present in the chordae tendineae 1110 where the leaflets 1108 are even longer relative to the distal anchors 80. The leaflet 1108 can be sufficiently long such that the distal anchors 80 do not contact the annulus 1106.

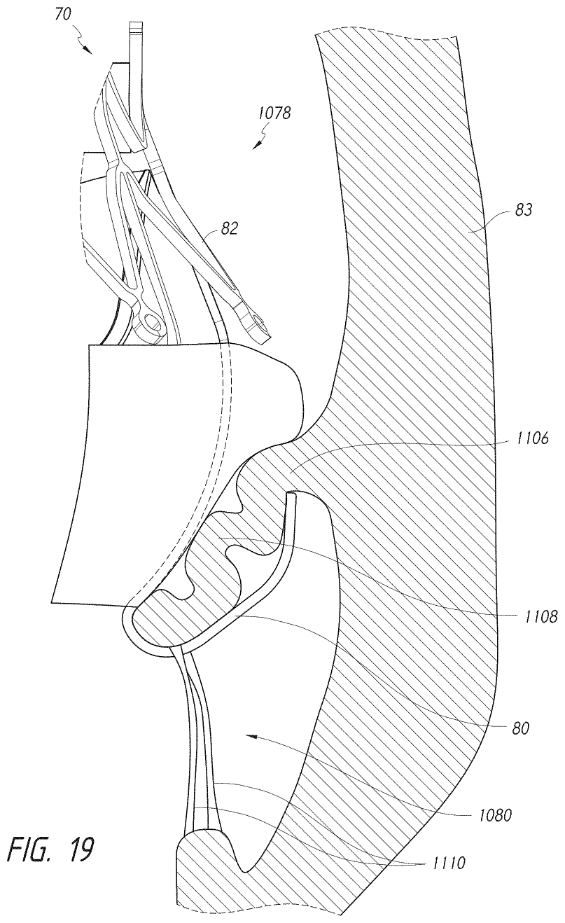

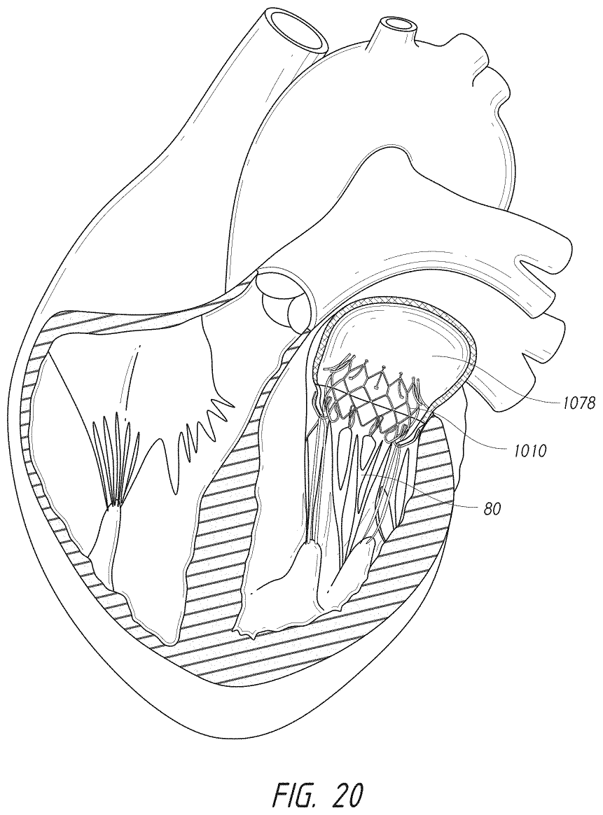

The proximal anchors 82, if present, can be positioned such that the ends or tips of the proximal anchors 82 are adjacent the atrial side of the annulus 1106 and/or tissue of the left atrium 1078 beyond the annulus 1106. In some situations, some or all of the proximal anchors 82 may only occasionally contact or engage atrial side of the annulus 1106 and/or tissue of the left atrium 1078 beyond the annulus 1106. For example, as illustrate in FIG. 19, the proximal anchors 82 may be spaced from the atrial side of the annulus 1106 and/or tissue of the left atrium 1078 beyond the annulus 1106. The proximal anchors 82 could provide axial stability for the prosthesis 70. It is also contemplated that some or all of the proximal anchors 82 may contact the atrial side of the annulus 1106 and/or tissue of the left atrium 1078 beyond the annulus 1106. FIG. 20 illustrates the prosthesis 70 implanted in the heart. Although the illustrated replacement heart valve includes both proximal and distal anchors, it will be appreciated that proximal and distal anchors are not required in all cases. For example, a replacement heart valve with only distal anchors may be capable of securely maintaining the replacement heart valve in the annulus. This is because the largest forces on the replacement heart valve are directed toward the left atrium during systole. As such, the distal anchors are most important for anchoring the replacement heart valve in the annulus and preventing migration.

### Delivery Method

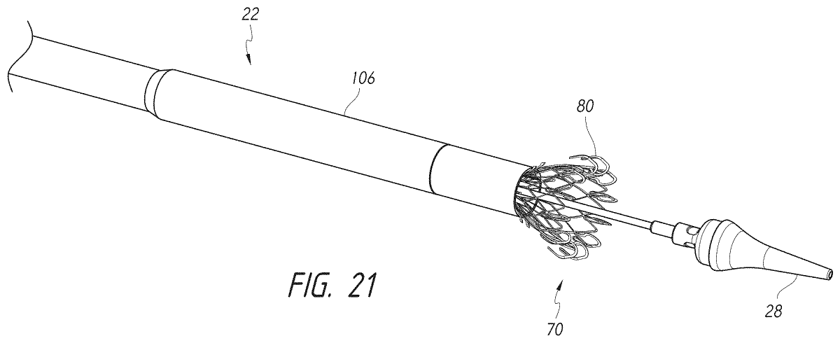

FIGS. 21-23 illustrate the release mechanism of the delivery system 10. During the initial insertion of the prosthesis 70 and the delivery system 10 into the body, the prosthesis 70 can be located within the system 10, similar to as shown in FIG. 2A. The distal end 303 of the prosthesis 70, and specifically the distal anchors 80, are restrained within the capsule 106 of the outer sheath assembly 22, thus preventing expansion of the prosthesis 70. Similar to what is shown in FIG. 2A, the distal anchors 80 can extend distally when positioned in the capsule. The proximal end 301 of the prosthesis 70 is restrained within the capsule 106 and within a portion of the inner retention member 40 and thus is generally constrained between the capsule 106 and the inner retention member 40.

The system 10 can first be positioned to a particular location in a patient's body, such as at the native mitral valve, through the use of the steering mechanisms discussed herein or other techniques.

Once the prosthesis 70 is loaded into the delivery system 10, a user can thread a guide wire into a patient to the desired location. The guide wire passes through the lumen of the nose cone assembly 31, and thus the delivery system 10 can be generally advanced through the patient's body following the guide wire. The delivery system 10 can be advanced by the user manually moving the handle 14 in an axial direction. In some embodiments, the delivery system 10 can be placed into a stand while operating the handle 14 controls.

Once generally in heart, the user can begin the steering operation of the rail assembly 20 using the distal pull wire knob 206 and/or the proximal pull wire knob 208. By turning either of the knobs, the user can provide flexing/bending of the rail assembly 20 (either on the distal end or the proximal end), thus bending the distal end of the delivery system 10 in one, two, or more locations into the desired configuration. As discussed above, the user can provide multiple bends in the rail assembly 20 to direct the delivery system 10 towards the mitral valve. In particular, the bends of the rail assembly 20 can direct a distal end of the delivery system 10, and thus the capsule 106, along the center axis passing through the native mitral valve. Thus, when the outer sheath assembly 22, mid shaft assembly 21, inner assembly 18, and nose cone assembly 31 are together advanced over the rail assembly 20 with the compressed prosthesis 70, the capsule 106 proceed directly in line with the axis for proper release of the prosthesis 70.

The user can also rotate and/or move the handle 14 itself in a stand for further fine tuning of the distal end of the delivery system 10. The user can continually turn the proximal and/or distal pull wire knobs 208/206, as well as moving the handle 14 itself, to orient the delivery system 10 for release of the prosthesis 70 in the body. The user can also further move the other assemblies relative to the rail assembly 20, such as proximally or distally.

In a next step, the user can rotate the depth knob 212. As discussed, rotation of this knob 212 together advances the inner shaft assembly 18, mid shaft assembly 21, outer sheath assembly 22, and nose cone assembly 31 over/through the rail assembly 20 while the prosthesis 70 remains in the compressed configuration within the implant retention area 16. Due to the rigidity of, for example, either the inner shaft assembly 18, the mid shaft assembly 21, and/or the outer sheath assembly 22, these assemblies proceed straight forward in the direction aligned by the rail assembly 20.

Once in the release position, the user can rotate the outer sheath knob 210, which individually translates the outer sheath assembly 22 (and thus the capsule 106) with respect to the other assemblies, in particular the inner assembly 18, in a proximal direction towards the handle 14 as shown in FIG. 21. By doing so, the distal end 303 of prosthesis 70 is uncovered in the body, allowing for the beginning of expansion. At this point, the distal anchors 80 can flip proximally and the distal end 303 begins to expand radially outwardly. For example, if the system 10 has been delivered to a native mitral valve location through a transseptal approach, the nose cone is positioned in the left ventricle, preferably aligning the prosthesis 70 such that it is generally perpendicular to the plane of the mitral annulus. The distal anchors 80 expand radially outwardly within the left ventricle. The distal anchors 80 can be located above the papillary heads, but below the mitral annulus and mitral leaflets. In some embodiments, the distal anchors 80 may contact and/or extend between the chordae in the left ventricle, as well as contact the leaflets, as they expand radially. In some embodiments, the distal anchors 80 may not contact and/or extend between the chordae or contact the leaflets. Depending on the position of the prosthesis 70, the distal ends of the distal anchors 80 may be at or below where the chordae connect to the free edge of the native leaflets.

As shown in the illustrated embodiment, the distal end 303 of the prosthesis 70 is expanded outwardly. It should be noted that the proximal end 301 of the prosthesis 70 can remain covered by the outer retention ring during this step such that the proximal end 301 remains in a radially compacted state. At this time, the system 10 may be withdrawn proximally so that the distal anchors 80 capture and engage the leaflets of the mitral valve, or may be moved proximally to reposition the prosthesis 70. For example, the assemblies may be proximally moved relative to the rail assembly 20. Further, the system 10 may be torqued, which may cause the distal anchors 80 to put tension on the chordae through which at least some of the distal anchors may extend between. However, in some embodiments the distal anchors 80 may not put tension on the chordae. In some embodiments, the distal anchors 80 may capture the native leaflet and be between the chordae without any further movement of the system 10 after withdrawing the outer sheath assembly 22.

During this step, the system 10 may be moved proximally or distally to cause the distal or ventricular anchors 80 to properly capture the native mitral valve leaflets. This can be done by moving the outer sheath assembly 22, mid shaft assembly 21, inner assembly 18, and nose cone assembly 31 with respect to the rail assembly 20. In particular, the tips of the ventricular anchors 80 may be moved proximally to engage a ventricular side of the native annulus, so that the native leaflets are positioned between the anchors 80 and the body of the prosthesis 70. When the prosthesis 70 is in its final position, there may or may not be tension on the chordae, though the distal anchors 80 can be located between at least some of the chordae.

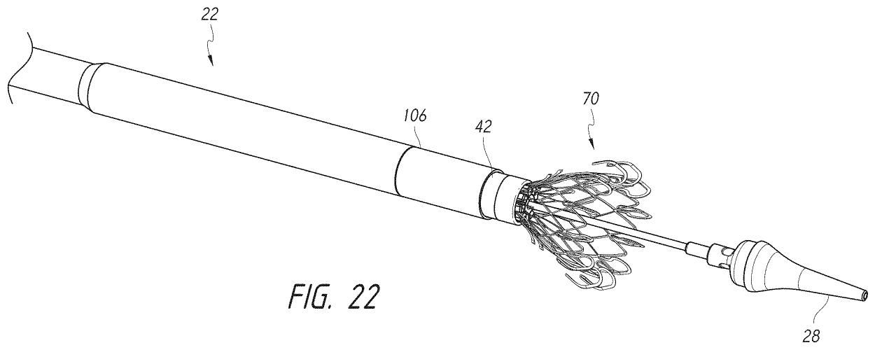

The proximal end 301 of the prosthesis 70 will remain in the outer retention ring 42 after retraction of the capsule 106. As shown in FIG. 22, once the distal end 303 of the prosthesis 70 is fully expanded (or as fully expanded as possible at this point), the outer retention ring 42 can be individually withdrawn proximally with respect to the other assemblies, in particular relative to the inner assembly 18, to expose the inner retention member 40, thus beginning the expansion of the proximal end 301 of the prosthesis 70. For example, in a mitral valve replacement procedure, after the distal or ventricular anchors 80 are positioned between at least some of the chordae tendineae and/or engage the native mitral valve annulus, the proximal end 301 of the prosthesis 70 may be expanded within the left atrium.

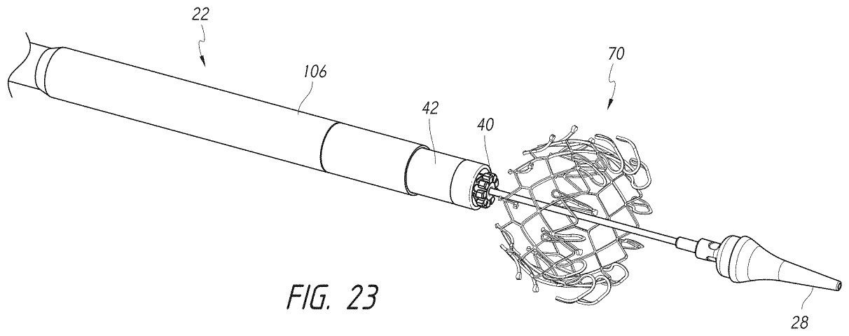

The outer retention ring 42 can be moved proximally such that the proximal end 310 of the prosthesis 70 can radially expand to its fully expanded configuration as shown in FIG. 23. After expansion and release of the prosthesis 70, the inner assembly 18, nose cone assembly 31, mid shaft assembly 21, and outer sheath assembly 22 can be simultaneously withdrawn proximally along or relative to the rail assembly 20 back to their original position. In some embodiments, they are not withdrawn relative to the rail assembly 20 and remain in the extended position. Further, the nose cone 28 can be withdrawn through the center of the expanded prosthesis 70 and into the outer sheath assembly 22, such as by proximally translating the knob 216. The system 10 can then be removed from the patient.

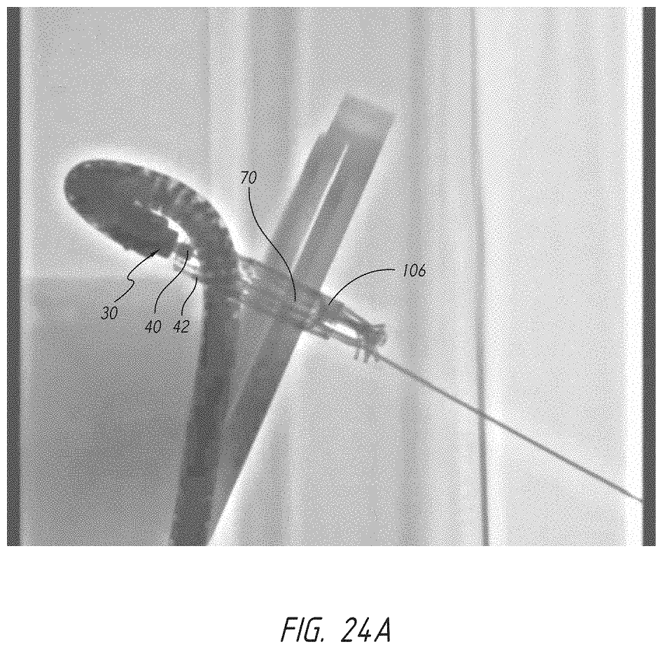

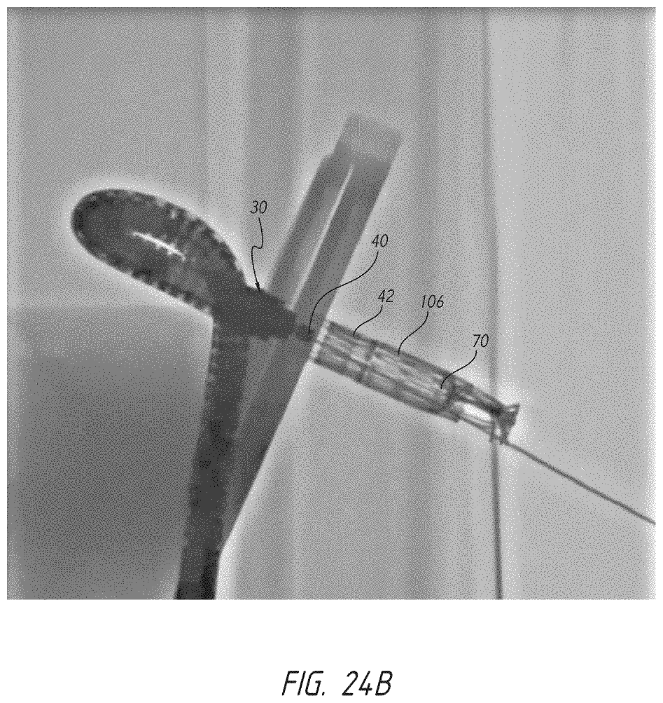

FIGS. 24A-B illustrate the advancement of the different assemblies over the rail assembly 20. FIG. 24A illustrates the assemblies in their proximalmost position over the rail assembly 20. FIG. 24B illustrates the assemblies in their distalmost position as compared to the rail assembly 20, such as shown in FIG. 2C. Thus, the assemblies snake along the rail assembly 20 and extend distally away.

In some embodiments, the prosthesis 70 can be delivered under fluoroscopy so that a user can view certain reference points for proper positioning of the prosthesis 70. Further, echocardiography can be used for proper positioning of the prosthesis 70.

Following is a discussion of an alternative implantation method for delivering a replacement mitral valve to a mitral valve location. Elements of the below can be incorporated into the above discussion and vice versa. Prior to insertion of the delivery system 10, the access site into the patient can be dilated. Further, a dilator can be flushed with, for example, heparinized saline prior to use. The delivery system 10 can then be inserted over a guide wire. In some embodiments, any flush ports on the delivery system 10 can be pointed vertically. Further, if an introducer tube is used, integrated or otherwise, this can be stabilized. The delivery system 10 can be advanced through the septum until a distal end of the delivery system 10 is positioned across the septum into the left atrium 1078. Thus, the distal end of the delivery system 10 can be located in the left atrium 1078. In some embodiments, the delivery system 10 can be rotated, such as under fluoroscopy, into a desired position. The rail can be flex so that direct a distal end of the delivery system 10 towards the septum and mitral valve. The position of the delivery system 10, and the prosthesis 70 inside, can be verified using echocardiography and fluoroscopic guidance.

In some embodiments, the prosthesis 70 can be located, prior to release, above the mitral annulus 1106, in line with the mitral annulus 1106, or below the mitral annulus 1106. In some embodiments, the prosthesis 70 can be located, prior to expansion, fully above the mitral annulus 1106, in line with the mitral annulus 1106, just below the mitral annulus 1106, or fully below the mitral annulus 1106. In some embodiments, the prosthesis 70 can be located, prior to expansion, partially above the mitral annulus 1106, in line with the mitral annulus 1106, or partially below the mitral annulus 1106. In some embodiments, a pigtail catheter can be introduced into the heart to perform a ventriculogram for proper viewing.

In some embodiments, the position of the mitral plane and the height of any papillary muscles on the fluoroscopy monitor can be marked to indicate an example target landing zone. If needed, the delivery system 10 can be unflexed, reduced in rotation, and retracted to reduce tension on the delivery system 10 as well as reduce contact with the left ventricular wall, the left atrial wall, and/or the mitral annulus 1106.

Further, the delivery system 10 can be positioned to be coaxial to the mitral annulus 1106, or at least as much as possible, while still reducing contact with the left ventricular wall, the left atrial wall, and/or the mitral annulus 1106 and reducing delivery system tension. An echo probe can be positioned to view the anterior mitral leaflet (AML), the posterior mitral leaflet (PML) (leaflets 1108), mitral annulus 1106, and outflow tract. Using fluoroscopy and echo imaging, the prosthesis 1010 can be confirmed to be positioned at a particular depth and coaxiality with the mitral annulus 1106.

Afterwards, the outer sheath assembly 22 can be retracted to expose the ventricular anchors 80, thereby releasing them. In some embodiments, once exposed, the outer sheath assembly 22 can be reversed in direction to relieve tension on the outer sheath assembly 22. In some embodiments, reversing the direction could also serve to partially or fully capture the prosthesis 70.

The distal anchors 80 can be released in the left atrium 1078. Further, the proximal anchors 82, if included in the prosthesis 70, are not yet exposed. Moreover, the body of the prosthesis 70 has not undergone any expansion at this point. However, in some embodiments, one or more of the distal anchors 80 can be released in either the left atrium 1078 (e.g., super-annular release) or generally aligned with the mitral valve annulus 1106 (e.g., intra-annular release), or just below the mitral valve annulus 1106 (e.g., sub-annular release). In some embodiments, all of the distal anchors 80 can be released together. In other embodiments, a subset of the distal anchors 80 can be released while at a first position and another subset of the distal anchors 80 can be released while at a second position. For example, some of the distal anchors 80 can be released in the left atrium 1078 and some of the distal anchors 80 can be released while generally aligned with the mitral valve annulus 1106 or just below the mitral valve annulus 1106.

If the distal anchors 80 are released “just below” the mitral valve annulus 1106, the may be released at 1 inch, ¾ inch, ½ inch, ¼ inch, ⅛ inch, 1/10 inch or 1/20 inch below the mitral valve annulus 1106. In some embodiments, the distal anchors 80 the may be released at less than 1 inch, ¾ inch, ½ inch, ¼ inch, ⅛ inch, 1/10 inch or 1/20 inch below the mitral valve annulus 1106. This may allow the distal anchors 80 to snake through the chordae upon release. This can advantageously allow the prosthesis 70 to slightly contract when making the sharp turn down toward the mitral valve. In some embodiments, this may eliminate the need for a guide wire assisting to cross the mitral valve. In some embodiments, the guide wire may be withdrawn into the delivery system 10 before or following release of the distal anchors 80.

In some embodiments, the distal anchors 80 can be released immediately after crossing the septum, and then the final trajectory of the delivery system 10 can be determined. Thus, the delivery system 10 can cross the septum, release the ventricular anchors 80, establish a trajectory, and move into the left ventricle to capture the leaflets.

As discussed in detail above, upon release from the delivery system 10, the distal anchors 80 can flip from extending distally to extending proximally. This flip can be approximately 180°. Accordingly, in some embodiments, the distal anchors 80 can be flipped in either the left atrium 1078 (e.g., super-annular flip), generally aligned with the mitral valve annulus 1106 (e.g., intra-annular flip), or just below the mitral valve annulus 1106 (e.g., sub-annular flip). The proximal anchors 82, if any, can remain within the delivery system 10. In some embodiments, all of the distal anchors 80 can be flipped together. In other embodiments, a subset of the distal anchors 80 can be flipped while at a first position and another subset of the distal anchors 80 can be released while at a second position. For example, some of the distal anchors 80 can be flipped in the left atrium 1078 and some of the distal anchors 80 can be flipped while generally aligned with the mitral valve annulus 1106 or just below the mitral valve annulus 1106.

In some embodiments, the distal anchors 80 may be positioned in line with the annulus 1106 or just below the annulus 1106 in the non-flipped position. In some embodiments, the distal anchors 80 may be position in line with the annulus 1106 or just below the annulus 1106 in the flipped position. In some embodiments, prior to flipping the distalmost portion of the prosthesis 70 can be located within or below the mitral valve annulus 1106, such as just below the mitral valve annulus 1106. However, flipping the anchors can cause, without any other movement of the delivery system 10, the distalmost portion of the prosthesis 70/anchors 80 to move upwards, moving it into the left atrium 1078 or moving it in line with the mitral annulus 1106. Thus, in some embodiments the distal anchors 80 can begin flipping at the annulus 1106 but be fully within the left atrium 1078 upon flipping. In some embodiments the distal anchors 80 can begin flipping below the annulus 1106 but be fully within the annulus 1106 upon flipping.

In some embodiments, the distal anchors 80 can be proximal (e.g., toward the left atrium 1078) of a free edge of the mitral leaflets 1108 upon release and flipping. In some embodiments, the distal anchors 80 can be aligned with (e.g., toward the left atrium 1078) a free edge of the mitral leaflets 1108 upon release and flipping. In some embodiments, the distal anchors 80 can be proximal (e.g., toward the left atrium 1078) of a free edge of the mitral valve annulus 1106 upon release and flipping. In some embodiments, the distal anchors 80 can be aligned with (e.g., toward the left atrium 1078) a free edge of the mitral valve annulus 1106 upon release and flipping.

Thus, in some embodiments the distal anchors 80 can be released/flipped above where the chordae 1110 attach to the free edge of the native leaflets 1108. In some embodiments the distal anchors 80 can be released/flipped above where some the chordae 1110 attach to the free edge of the native leaflets 1108. In some embodiments the distal anchors 80 can be released/flipped above where all the chordae 1110 attach to the free edge of the native leaflets 1108. In some embodiments, the distal anchors 80 can be released/flipped above the mitral valve annulus 1106. In some embodiments, the distal anchors 80 can be released/flipped above the mitral valve leaflets 1108. In some embodiments, the distal anchors 80 can be released/flipped generally in line with the mitral valve annulus 1106. In some embodiments, the distal anchors 80 can be released/flipped generally in line with the mitral valve leaflets 1108. In some embodiments, the tips of the distal anchors 80 can be released/flipped generally in line with the mitral valve annulus 1106. In some embodiments, the tips of the distal anchors 80 can be released/flipped generally in line with the mitral valve leaflets 1108. In some embodiments the distal anchors 80 can be released/flipped below where some the chordae 1110 attach to the free edge of the native leaflets 1108. In some embodiments the distal anchors 80 can be released/flipped below where all the chordae 1110 attach to the free edge of the native leaflets 1108. In some embodiments, the distal anchors 80 can be released/flipped below the mitral valve annulus 1106. In some embodiments, the distal anchors 1024 can be released/flipped below the mitral valve leaflets 1108.

Once the distal anchors 80 are released and flipped, the delivery system 10 can be translated towards the left ventricle 1080 through the mitral valve annulus 1106 so that the distal anchors 80 enter the left ventricle 1080. In some embodiments, the distal anchors 80 can compress when passing through the mitral valve annulus 1106. In some embodiments, the prosthesis 70 can compress when passing through the mitral valve annulus 1106. In some embodiments, the prosthesis 70 does not compress when it passes through the mitral annulus 1106. The distal anchors 80 can be delivered anywhere in the left ventricle 1080 between the leaflets 1108 and the papillary heads.

In some embodiments, the distal anchors 80 are fully expanded prior to passing through the mitral valve annulus 1106. In some embodiments, the distal anchors 80 are partially expanded prior to passing through the mitral valve annulus 1106 and continued operation of the delivery system 10 can fully expand the distal anchors 80 in the left ventricle 1080.

When the distal anchors 80 enter the left ventricle 1080, the distal anchors 80 can pass through the chordae 1110 and move behind the mitral valve leaflets 1108, thereby capturing the leaflets 1108. In some embodiments, the distal anchors 80 and/or other parts of the prosthesis 1010 can push the chordae 1110 and/or the mitral valve leaflets 1108 outwards.

Thus, after release of the distal anchors 80, the delivery system 10 can then be repositioned as needed so that the ends of the left distal anchors 80 are at the same level of the free edge of the native mitral valve leaflets 1108. The delivery system 10 can also be positioned to be coaxial to the mitral annulus 1106 if possible while still reducing contact with the left ventricular wall, the left atrial wall, and/or the annulus 1106.

In some embodiments, only the distal anchors 80 are released in the left atrium 1078 before the prosthesis 70 is move to a position within, or below, the annulus. In some alternate embodiments, the distal end of the prosthesis 70 can be further expanded in the left atrium 1078. Thus, instead of the distal anchors 80 flipping and no portion of the prosthesis 70 body expanding, a portion of the prosthesis 70 can be exposed and allowed to expand in the left atrium 1078. This partially exposed prosthesis 1010 can then be passed through the annulus 1106 into the left ventricle 1080. Further, the proximal anchors, if any, can be exposed. In some embodiments, the entirety of the prosthesis 70 can be expanded within the left atrium 1078.

To facilitate passage through the annulus 1106, the delivery system 10 can include a leader element (not shown) which passes through the annulus 1106 prior to the prosthesis 70 passing through the annulus 1106. For example, the leader element can include an expandable member, such as an expandable balloon, which can help maintain the shape, or expand, the annulus 1106. The leader element can have a tapered or rounded shape (e.g., conical, frustoconical, semispherical) to facilitate positioning through and expansion of the annulus 1106. In some embodiments, the delivery system 10 can include an engagement element (not shown) which can apply a force on the prosthesis 70 to force the prosthesis 70 through the annulus 1106. For example, the engagement element can include an expandable member, such as an expandable balloon, positioned within or above the prosthesis 70.

In some embodiments, to facilitate passage through the annulus 1106, a user can re-orient the prosthesis 70 prior to passing the prosthesis 70 through the annulus 1106. For example, a user can re-orient the prosthesis 70 such that it passes through the annulus 1106 sideways.

However, if only the distal anchors 80 are flipped, and no other expansion occurs, the prosthesis can be partially expanded in the ventricle 1080. Thus, when the prosthesis 70 is in the proper location, the distal end can be allowed to expand to capture the leaflets 1108. If the distal end is already expanded, no more expansion may take place or the distal end can be further expanded.

Further, the PML and AML 1106 can be captured, for example by adjusting the depth and angle of the prosthesis 70. If a larger prosthesis diameter is needed to capture the leaflets 1106, the outer sheath assembly 22 can be retracted until the desired diameter of the prosthesis 70 is achieved. Capture of the leaflets 1106 can be confirmed through echo imaging. In some embodiments, a user can confirm that the prosthesis 70 is still in the appropriate depth and has not advanced into the left ventricle 1080. The position can be adjusted as needed.

In some embodiments, once the distal anchors 80 enter the left ventricle 1080 the system 10 can be pulled backwards (e.g., towards the left atrium 1078) to fully capture the leaflets 1108. In some embodiments, the system 10 does not need to be pulled backwards to capture the leaflets 1108. In some embodiments, systolic pressure can push the leaflets 1108 upwards to be captured by the distal anchors 80. In some embodiments, systolic pressure can push the entire prosthesis 70 up towards the mitral annulus 1106 after the leaflets 1108 are captured and the prosthesis 70 is fully or partially released. In some embodiments, a user can rotate the delivery system 10 and/or prosthesis 70 prior to and/or while pulling the delivery system 10 backwards. In some instances, this can beneficially engage a greater number of chordae tendineae.

The outer sheath assembly 22 can be further retracted to fully expand the prosthesis. Once the prosthesis 70 is fully exposed, the delivery system 10 can be maneuvered to be coaxial and height relative to the mitral annulus 1106, such as by flexing, translating, or rotating the delivery system 10. As needed, the prosthesis 70 can be repositioned to capture the free edge of the native mitral valve leaflets 1108. Once full engagement of the leaflets 1108 is confirmed, the prosthesis 70 can be set perpendicular (or generally perpendicular) to the mitral annular plane.

Following, the mid shaft assembly 21 can be withdrawn. The mid shaft assembly 21 can then be reversed in direction to relieve any tension on the delivery system 10.

Below is a discussion of proximal anchors 82, though some embodiments of the prosthesis 70 may not include them. In some embodiments, proximal anchors 82 may not be released from the system 10 until the distal anchors 80 have captured the leaflets 1108. In some embodiments, proximal anchors 82 may be released from the system 10 prior to the distal anchors 80 capturing the leaflets 1108. In some embodiments, the proximal anchors 82 can be released when the distal anchors 80 are super or intra annular and the expanded prosthesis 70 (either partially or fully expanded) can be translated through the mitral annulus 1106. In some embodiments, the proximal anchors 82 could be released when the distal anchors 80 are sub-annular and the entire prosthesis 70 can be pulled up into the left atrium 1078 such that the proximal anchors 82 are supra-annular prior to release. In some embodiments, the proximal anchors 82 could be intra-annular prior to release and the systolic pressure could push the prosthesis 70 atrially such that the proximal anchors 82 end up supra-annular.

After, the leaflet capture and positioning of the prosthesis 70 can be confirmed, along with the relatively perpendicular position with respect to the mitral annular plane. In some embodiments, the nosecone 28 can then be withdrawn until it is within the prosthesis 70. The mid shaft assembly 21 can be further retracted until the prosthesis 70 is released from the delivery system 10. Proper positioning of the prosthesis 70 can be confirmed using TEE and fluoroscopic imaging.

Following, the delivery system 10 can be centralized within the prosthesis 70. The nosecone 28 and delivery system 10 can then be retracted into the left atrium 1078 and removed.

This intra-super annulus release can have a number of advantages. For example, this allows the distal anchors 82 to be properly aligned when contacting the chordae 1110. If the distal anchors 82 were released in the left ventricle 1080, this could cause misalignment or damage to heart tissue, such as the leaflets 1108 or chordae 1110.

In an alternate delivery approach, the delivery system 10 can be translated into the left ventricle 1080 prior to release of the prosthesis 70. Thus, the distal end of the prosthesis 70, and thus the distal anchors 82, can be released and flipped partially, or fully within the left ventricle 1080. Accordingly, in some embodiments the anchors 70 can be released/flipped below the mitral annulus 1106, just below the mitral annulus 1106, and/or below the free edges of the leaflets 1108. Further, the anchors 70 can be released above the papillary heads. Similar methodology as discussed above can then be used to properly position the prosthesis 70 and remove the delivery system 10 to deliver the prosthesis 1010. Further, in some embodiments the distal anchors 82 can be released without expanding the prosthesis initially in the ventricle 1080.

### Planetary Handle Gear

As discussed above, the delivery system 10 can include multiple shafts and one or more pull wires, some or all of which may be translated longitudinally/axially with respect to one another. However, having more movable components requires some sort of axial translation, which means that the handle 14 tends to become longer. This occurs because the different components within the handle 14 are generally moving in series, and thus there must be sufficient longitudinal space between handle components.

In order to prevent further lengthening of the handle 14, or even to provide a reduction in size of the handle 14, a planetary gear system can be used in the handle 14. This planetary handle gear system can be used in conjunction with, or instead of, the current linear screws in the delivery system 10. Embodiments of the planetary gears are shown in FIGS. 25A and 25B, wherein the components can be located within a handle housing, such as handle 14. By incorporating the disclosed planetary gears, rotary motion can be converted into linear motion in such a way that it allows room for multiple linear translation members to occupy the same area in the handle 14, thereby shortening the handle design. Thus, instead of the different components being in series, they are in parallel. The disclosed planetary gear system can allow knobs or other rotatable actuating mechanisms on the handle 14 that control axial movement or bending of various shafts or lumens of the delivery system 10 to be placed much closer to one another.

In some embodiments, the handle 14 may include one or more outermost gears (e.g., ring gear 2502), which can be connected to a rotatable knob on the handle 14 such as disclosed above. The ring gear 2502 can encompass an entire circumference or substantially an entire circumference of the handle 14 and thus be connected directly to a knob. Alternatively, the ring gear 2502 may be smaller than the handle 14 and connected to a knob by one or more intermediate components.

Further, the handle 14 can include one or more planet gears 2504 within the ring gear 2502. The planet gears 2504 can be smaller in circumference/diameter than the ring gear 2502 and may be interlocked within an inner surface of the ring gear 2504. The planet gear 2504 can be axially and circumferentially fixed in place, though in some embodiments it may not be. If fixed in place, the planet gear 2504 is only allowed to rotate in place by rotation of the ring gear 2504, and thus does not translate around a circumference of the ring gear 2502. This can prevent any components attached to the gear system from circumferentially moving around the ring gear 2504.

Additionally, the planet gear 2504 can have an internal thread which can mate with a linear travel screw 2506, which can be attached to any of the axially translating components (e.g., shaft assemblies such as the outer sheath assembly 22, the mid shaft assembly 21, the inner assembly 18, and/or the nose cone assembly 31) discussed above. Thus, as the ring gear 2502 is rotated by the handle 14, the ring gear 2502 will rotate the planet gear 2504 in place, which will each in turn translate the linear travel screw 2506 in an axial direction. Thus, the rotational motion of the ring gear 2502 can be converted into linear translation of the travel screw 2506, providing the linear translation discussed above.

As shown in FIGS. 25A and 25B, the planetary gear system could control multiple planet gears 2504 of the ring gear 2502. In some embodiments, the handle 14 can contain multiple planetary gear systems, such as a 2, 3, 4, or 5 gear system. In some embodiments, each ring gear 2502 can include 1, 2, 3, 4, 5, or 6 planet gears 2504.

### Valve Deployment Simulation Catheter

As discussed above, the delivery system 10 (or other delivery system) can include a rigid component attached to the prosthesis 70 prior to full implant deployment. For example, the prosthesis 70 generally surrounds nose cone shaft 27, which can be a generally rigid shaft. When still partially or fully within the delivery system 10, the prosthesis 70 may be forced into the proper position by the delivery system 10, which may be against the natural anatomy of the valve area. However, the prosthesis 70 can jump or move inside the patient's anatomy once detached from the delivery system 10, such as when uncovered by the outer retention ring 42 or capsule 106. This can occur because when the prosthesis 70 is removed from the delivery system 10, it loses the support of the delivery system 10 and moves to a non-resistant position inside the natural valve anatomy.

Thus, if the prosthesis 70 is not co-axial with the native anatomy, or perpendicular to the native annulus, when the prosthesis 70 is deployed, the prosthesis 70 loses the rigid support the delivery system 10 is exerting. If the prosthesis 70 does not capture the leaflets, then the valve can move into a position where a paravalvular leak forms around the prosthetic valve leaflets.

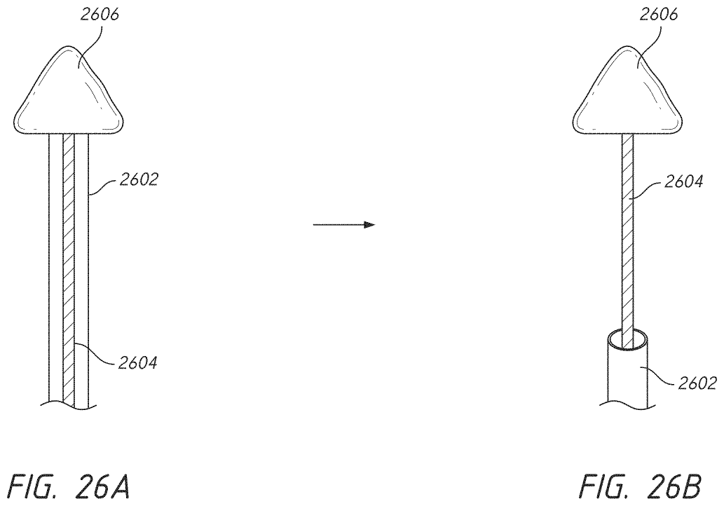

For example, as shown in FIGS. 26A-26B, the delivery system 10 can include both a rigid shaft 2602 and a bendable (e.g., flexible) shaft 2604 for connection to the nosecone 2606. These components can replace the nose cone shaft 27 discussed above. The rigid shaft 2602 can still be somewhat flexible but is just less bendable than the bendable shaft 2604.

As shown, the rigid shaft 2602 can surround the bendable shaft 2604 so that the bendable shaft 2604 extends within a lumen of the rigid shaft 2602. Thus, the rigid shaft 2602 prevents significant bending of the bendable shaft 2604. The rigid shaft 2602 can extend all the way to the nosecone 2606, or may extend only partially over the bendable shaft 2604. The bendable shaft 2604 may attach to the nosecone 2606 as discussed in conjunction with the nose cone shaft 27. The bendable shaft 2604 may be, for example, wires, cut shafts, a bendable polymer, or other materials and the particular material is not limiting.

As shown in FIG. 26A, while delivering the prosthesis 70, the rigid shaft 2602 may be in a fully distal position covering the bendable shaft 2604. Once in the desired position, the rigid shaft 2602 can be proximally withdrawn as shown in FIG. 26B and the prosthesis 70 may be partially released from the delivery system 10. Thus, the bendable shaft 2604 will allow the prosthesis 70 to conform with the natural patient anatomy while the prosthesis 70 is still attached to the delivery system 10. If the positioning is not proper, an operator can cover the prosthesis 70 and/or distally translate the rigid shaft 2602 and try another position. The bendable shaft 2604 can eliminate most of the rigid support the delivery system exerts on the prosthesis 70 once uncovered by the rigid shaft 2602.

In some embodiments, instead of using a rigid shaft 2602, the bendable shaft 2604 can transition from a “rigid” structure to a less rigid “bendable” structure. In some embodiments, the rigid shaft 2602 may be located within a lumen of the bendable shaft 2604.

### Locking Shaft Release

While the above-disclosed delivery system 10 includes a number of shafts and components that are fixed at their proximal end until movement of different actuators, such as at the handle 14, it can be advantageous for the components to be locked (or fixed) instead at their distal end. In order to engage/disengage with one another, the layers can be rotated relative to one another.

Specifically, the inner retention member 40 and the outer retention member 42 can be locked together until any motion is desired. In some embodiments, the members 40/42 can be threaded together. In some embodiments, the components can have additional locking features, which an operator can unlock when the operator wants to move the outer retention member 42 axially with respect to the inner retention member 40.

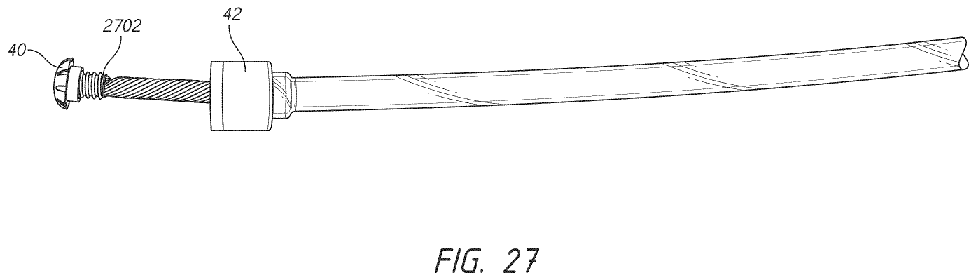

As shown in FIG. 27, the inner retention member 40 can include an outward (or outer or external) threading 2702 on its proximal end. Alternatively, or in conjunction with, the inner shaft 126 can include an outer threading. The outer retention member 42 can include an inner threading for attachment to the inner retention member 40. Alternatively, or in conjunction with any of the above configurations, the mid shaft 43 may include an inner threading.

Thus, an operator can translate the prosthesis 70 within the delivery system 10 to a location while the inner retention member 40 and the outer retention member 42 are locked together (e.g., threaded together). The operator can torque or rotate either one of the inner retention member 40 and the outer retention member 42, or both in some embodiments, in order to unlock the members 40/42 from one another. The operator can then axially withdraw the outer retention ring 42 to release the prosthesis 70. By connecting the two components at their distal ends, the risk of unintentional release is mitigated as the members 40/42 cannot move with respect to one another when locked together.

While the inner retention member 40 and the outer retention member 42 are disclosed with the torqueing features discussed herein, other components such as the rail assembly 20, nose cone assembly 31, or outer sheath assembly 22 may additionally include such features to connect different shaft assemblies.

### Spine Alignment Deflection Stabilization

In some embodiments, it can be advantageous to further include additional structural elements that can be configured to reduce deflection about a flex plane of certain shafts in the delivery system 10. This can improve the delivery system 10 under compressive loads by preventing uncontrolled deflection. For example, the delivery system 10 can inadvertently deflect in the direction of a flex plane during advancement of the delivery system 10 or when the delivery system 10 is under a compressive load, which can lead to procedural complications or accidental release of the prosthesis 70.

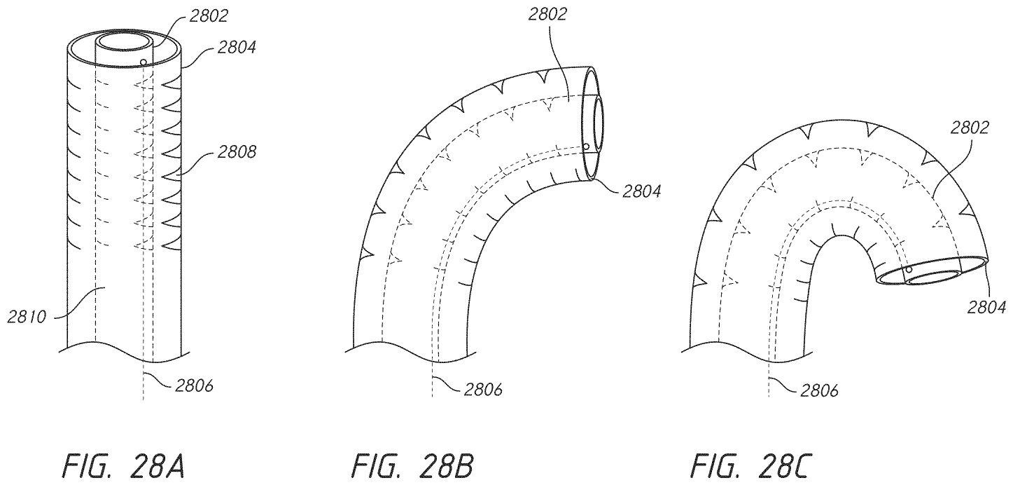

FIGS. 28A-28C illustrate a design which does not incorporate a stabilization feature as discussed herein. As shown, the spines 2810 of the different shafts 2802/2804 are aligned in FIG. 28A, so that when tension is applied to a pull wire 2806 the shafts 2802/2804 bend along the cut pattern 2808 as shown in FIG. 28B. However, if a compressive force is applied, the shafts 2802/2804 can transition into an uncontrolled bend as shown in FIG. 28C, which can lead to inadvertent release or movement of the prosthesis 70.

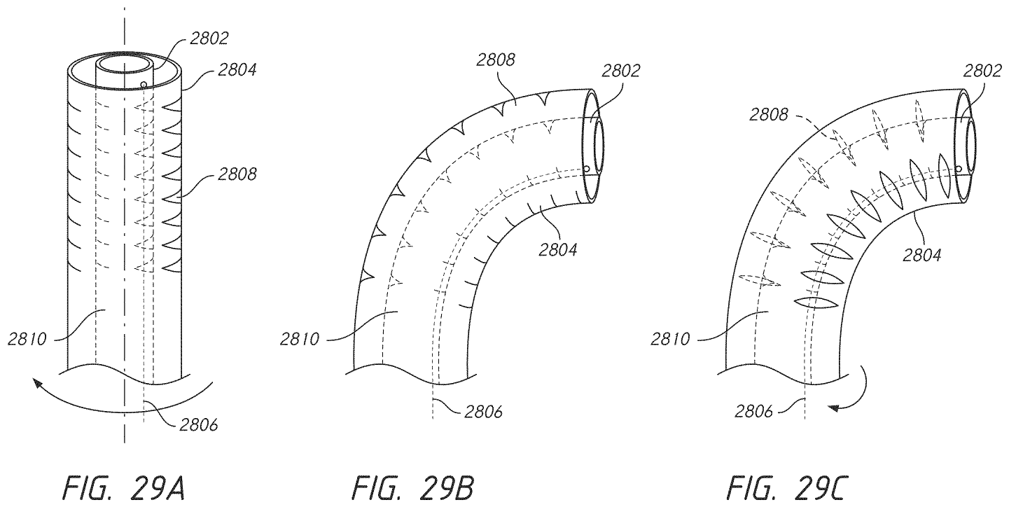

In order to avoid the uncontrolled bend shown in FIG. 28C, as shown in FIGS. 29A-29C, the delivery system 10 can allow for rotation of the different shafts 2802/2804 with respect to one another. Either one of the shafts 2802/2804 can be configured to rotate, or both can rotate. During delivery, the shafts 2802/2804 can be rotated with respect to one another so that the spine 2810 of one shaft (e.g., shaft 2802 is aligned with the cut pattern 2808 of another shaft (e.g., shaft 2804). Thus, when a load is applied, the shafts 2802/2804 stabilize each other, preventing uncontrolled flexing as shown in FIG. 28C, and instead only partially bend such as shown in FIG. 29C. When a flex is desired, the shafts 2802/2804 can be rotated so that the cut patterns 2808 align, allowing a pull wire 2806 to bend the shafts 2802/2804 in the desired direction as shown in FIG. 29B.

The above disclosed shaft configuration can be used in the inner assembly 18, mid shaft assembly 21, the rail assembly 20, nose cone assembly 31, and/or outer sheath assembly 22 and may additionally include such features to connect different shaft assemblies of other delivery systems not specifically illustrated herein.

### Keyed Delivery Components

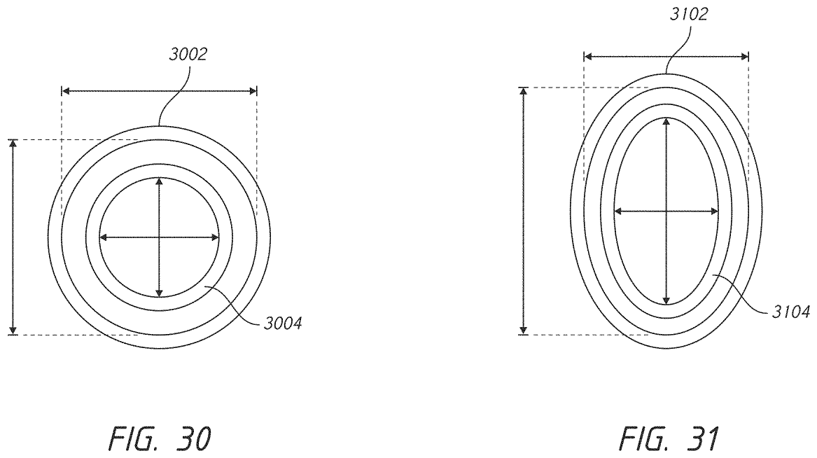

In some delivery systems, inner and outer shafts/lumens are not “keyed”, meaning that the shafts or lumens can rotate with respect to one another, as shown in FIG. 30. This can cause incidental (e.g., undesired) rotation of the shafts while using the delivery system 10 as an inner shaft 3004 can freely rotate within an outer shaft 3002 without constraint. To avoid this rotation, the handle 14 may utilize different connectors to prevent rotation. However, as the handle is at a proximal end of the shafts or lumens, this type of delivery system does not ensure that the two shafts are clocked or keyed along the lengths of the shafts all the way to the distal ends, or at least at the distal ends.

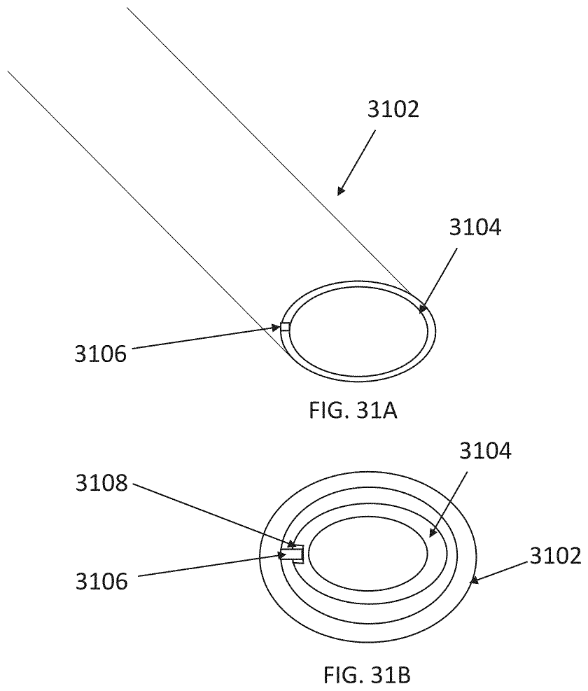

Accordingly, in some embodiments, the shafts or lumens (e.g., pull wire lumens 139 of the rail assembly 20) disclosed herein may be “keyed” as shown in FIGS. 31, 31A and 31B. The “keyed” configurations can prevent accidental (e.g., undesired, inadvertent, incidental) rotation of one shaft or lumen with respect to another shaft or lumen while still allowing translation (e.g., axial translation) with respect to each other. The keying can occur at a discrete portion of the shafts, such as at a distal end or a middle section, or may extend partially or fully along the length of the shafts.

As shown in FIGS. 31, 31A and 31B, in order to key two concentrically-arranged shafts or lumens together, one axis of the shafts 3102/3014 may be larger (e.g., have a longer length) than an orthogonal axis. The illustrated arrangement can form generally ovaloid lumens (e.g., eccentric or elliptical lumens), rather than a circular lumen as shown in FIG. 30. Thus, the inner shaft 3104 is prevented from rotating within the outer shaft 3102 through the physical constraint of the shape of the shafts (e.g., outer shaft 3102 and/or inner shaft 3104). While an ovaloid (e.g., eccentric or elliptical) configuration is disclosed, other configurations can be used as well, such as square or triangular shafts. In some embodiments, circular shafts can include internal components or external components that create an ovaloid shape, which can also prevent rotation while still allowing for relative translation.

Additionally, other keying embodiments can be used as well. For example, as illustrated in FIGS. 31A and 31B, one of the shafts (e.g., outer shaft 3102 as shown in the example of FIGS. 31A and 31B or inner shaft 3104) can include a tab 3106 which fits into a slot 3108 on another shaft (e.g., inner shaft 3104 as shown in the example of FIGS. 31A and 31B or outer shaft 3102), thereby preventing rotation. The particular keying structure is not limiting.

The above disclosed keying shaft configurations can be used in the inner assembly 18, mid shaft assembly 21, the rail assembly 20, nose cone assembly 31, and/or outer sheath assembly 22 or other shafts or lumens of a delivery system and may additionally include such features to connect different shaft assemblies or lumens. The keying shaft configurations can also be used in pull wire lumens (e.g., pull wire lumens 139 of the rail assembly 20) of the delivery system (e.g., delivery system 10).

### Valve Suture Locking and Release Mechanism

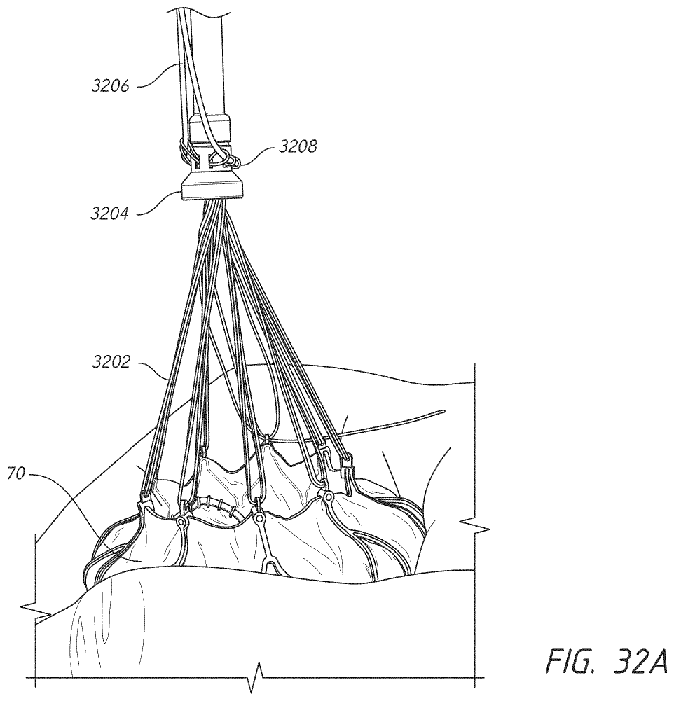

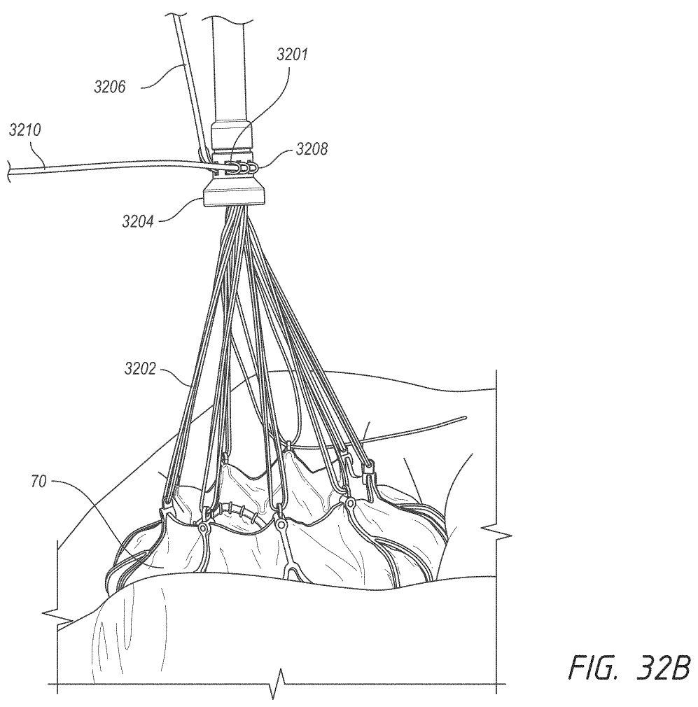

In some embodiments, tethers/sutures can be used as attachment mechanisms between a valve delivery system (e.g., delivery systems disclosed herein or delivery systems configured for delivery of replacement heart valves to replace an atrioventricular valve, a mitral valve, a tricuspid valve or other heart valve) instead of, or in conjunction with, the particular prosthesis 70 attachment mechanisms disclosed above. FIGS. 32A-36B illustrate embodiments of tether/suture attachment mechanisms.

As shown in FIG. 32A, tethers 3202 can extend from the prosthesis 70 (e.g., replacement heart valve) to a shaft having a manifold 3204 so that the prosthesis 70 can fully expand, such as using the methods disclosed above while still remaining connected to the delivery system 10 by the tethers 3202. The manifold 3204 can be used instead of the inner retention member 40 disclosed above. In some embodiments, both components can be used so that when the prosthesis 70 is released from the inner retention member 40, it stays attached to the delivery system 10 through the tethers 3202.

The manifold 3204 can include a number of apertures 3201 extending radially for loops 3208 (such as looped ends) of the tethers 3202 to extend through. Thus, the tethers 3202 can extend into an inner lumen of the manifold 3204 (e.g., from a distal direction) and partially extend through the apertures 3201 to an outer surface (e.g., radial or side surface) of the manifold 3204. A release (or locking) tether/suture 3206 can extend through the loops 3208, thus preventing the prosthesis (e.g., valve) 70 from being released from the manifold 3204 until ready.

The release tether 3206 can be withdrawn so that a free end 3210 of the release tether 3206 can pass through the loops 3208, thus releasing the prosthesis (e.g., valve) 70 from the tethered attachment to the manifold 3204, as shown in FIG. 32B. Multiple release (or locking) tethers/sutures 3206 may be used in some embodiments (e.g., one for each loop 3208 or one for multiple loops 3208).

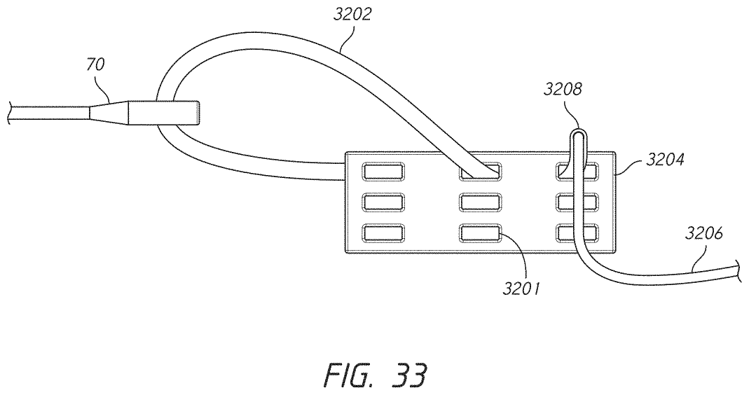

FIG. 33 illustrates an example of the manifold 3204 with apertures 3201. The number of apertures 3201 and the pattern of arrangement of apertures 3201 may vary as desired and/or required.

Advantageously, natural tension that the prosthesis 70 exerts on the suture loops 3208 when the prosthesis 70 is loaded also cinches the release (or locking) tether 3206, so there is no need for a catheter or user to keep a constant tension on the release (or locking) tether 3206. At a time when the prosthesis 70 is ready for release, a user may pull one end of the release (or locking) tether 3206 until it is unthreaded from all of the loops 3208, thereby enabling the loops 3208 to detach from the prosthesis 70 and the prosthesis 70 to thereby separate from the manifold 3204 of the delivery system.

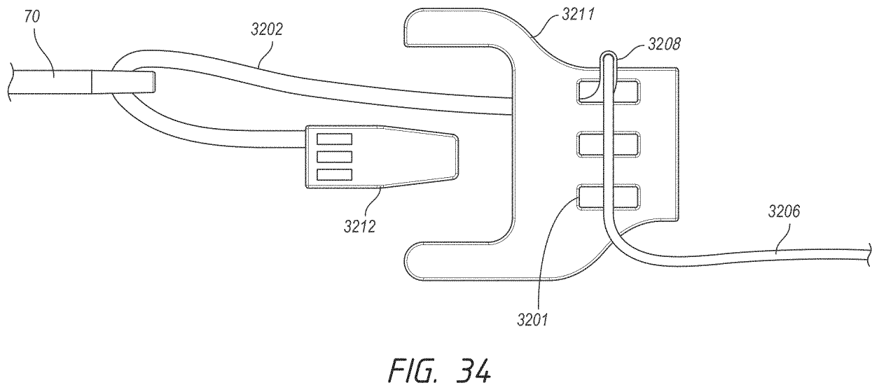

FIG. 34 illustrates an alternate embodiment of a tethered release system. As shown, a manifold combination, or assembly, can be composed of a modified outer retention member 3211 as well as modified inner retention member 3212. The tethers 3202 can have one end attached to the inner retention member 3212. The tethers 3202 can then pass through a portion of the prosthesis 70 (e.g., an eyelet extending from a proximal end of the prosthesis 70) and return back to the outer retention member 3211, where a second end of the tethers 3202 extends through the apertures 3201 and contains a loop 3208 for the release/locking tether 3206 to pass through. Members 3212/3211 can be moved relative to one another in order to tighten or loosen the tethers 3202. The tethers 3202 may comprise a single continuous strand or multiple separate strands.

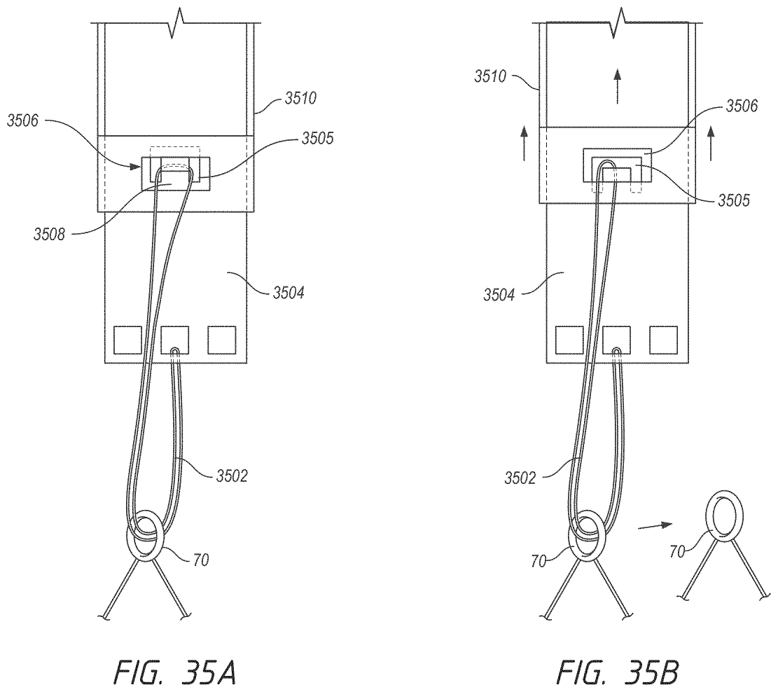

FIGS. 35A and 35B illustrate another embodiment of a suture attachment mechanism in locked (FIG. 35A) and unlocked (FIG. 35B) positions, or configurations. The suture attachment mechanism facilitates tethered attachment and release between a prosthesis (e.g., replacement heart valve) and a delivery system (e.g., delivery device or catheter). As shown, a tether 3502 (e.g., tether or suture loop) can be attached (e.g., non-removably attached or permanently attached via glue or other adhesive coupling) to a distal end of an inner shaft manifold 3504 and then can extend through a portion (e.g., an eyelet or other retention member) of a prosthesis (e.g., prosthesis 70) and then looped back to an outer shaft member 3510 (e.g., sleeve, shaft or lumen). The outer shaft member 3510 can include at least one radial aperture 3506. The inner manifold 3504 can include at least one aperture 3505 adapted to be aligned with the at least one radial aperture 3506 of the outer shaft member 3510, or vice-versa. The number of apertures 3505, 3506 may be the same or different. For example, the outer shaft member 3510 can have a single radial aperture 3506 extending around all or a portion of a circumference of the distal end portion of the outer shaft member 3510 and the inner manifold 3504 can have multiple circumferentially-spaced apart radial apertures 3505. As another example, the outer shaft member 3510 can include the same number of apertures as the inner manifold 3504 and corresponding apertures may be configured to align with each other circumferentially. The at least one aperture 3505 of the inner manifold 3504 includes a proximally- and/or radially outward-facing tooth 3508 extending from a distal edge of the aperture 3505 toward a proximal edge of the aperture 3505. The tether 3502 (e.g., one loop end of the tether 3502) can wrap around the tooth 3508. The outer shaft member 3510 of the manifold assembly can cover at least the proximal portion of the aperture 3505 in the locked position, or configuration, thereby preventing the tether 3502 (e.g., looped end of the tether 3502) from releasing (e.g., from being able to come off or be released from the tooth 3508) when in the locked position of FIG. 35A. The outer shaft member 3510 and/or the inner shaft manifold 3504 can be translated axially relative to each other. When the outer shaft member 3510 is proximally withdrawn or the inner manifold 3504 is distally advanced, to an unlocked configuration, the proximal end of tooth 3508 is exposed and the tether 3502 (e.g., looped end of the tether 3502) can be released from the tooth 3508, thereby facilitating decoupling of the prosthesis 70 from the manifold assembly of the delivery system while the tether 3502 remains attached to the inner manifold 3504. As the delivery system is withdrawn or removed from a desired implantation location (e.g., mitral valve annulus such as shown in FIG. 19 or 20, tricuspid valve annulus, atrioventricular valve annulus), the tether 3502 is pulled through the eyelet or other retention member of the prosthesis 70 such that the prosthesis 70 is no longer tethered, as shown schematically in FIG. 35B by the eyelet of the prosthesis 70 without a tether 3502. The suture attachment mechanism may include multiple tethers 3502 even though a single tether is shown for simplicity.

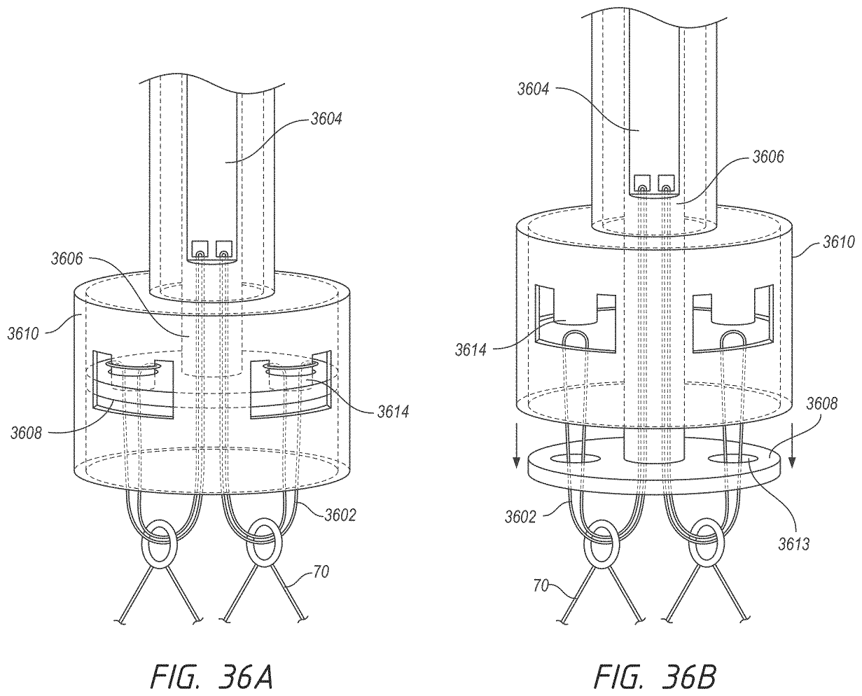

FIGS. 36A and 36B illustrate an alternative locking and release tether/suture mechanism with multiple lumens. As shown, sutures 3602 (e.g., suture or tether loops) can extend from an attachment point of an inner shaft 3604. The sutures 3602 can extend through the lumen of a middle shaft 3606 and out a distal end of the middle shaft 3606. The middle shaft 3606 can have a release disc 3608 on or within a distal end of the middle shaft 3606. The sutures 3602 can loop proximally (e.g., from a distal side of the release disc 3608) through apertures 3613 in the release disc 3608 and extend proximally to wrap around teeth 3614 of an outer ring 3610.

In the locked position, or configuration, shown in FIG. 36A, the disc 3608 can be proximally withdrawn to abut the teeth 3614, thereby preventing release of the sutures 3602. The disc 3608 can be advanced distally to allow for release of the tethers, or sutures 3602.

The tether/suture attachment mechanisms described herein can be used in conjunction with any prosthesis (e.g., replacement heart valve) that includes retention components (e.g., eyelets) through which one or more tethers can be looped. For example, the prosthesis could be a modified version of the prosthesis 70 illustrated in FIG. 3A that includes eyelets as shown in one or more of FIGS. 32A-36B instead of or in addition to the mushroom-shaped tabs 74. The prosthesis may be a replacement mitral valve, a replacement tricuspid valve, or other replacement heart valve. The tether/suture attachment mechanisms described herein (e.g., the manifold assemblies) may be incorporated into the delivery systems described herein (e.g., delivery system 10 or other delivery systems (e.g., transcatheter, transfemoral, transseptal delivery systems in conjunction with mitral valve replacement, tricuspid valve replacement, or other native heart valve replacement procedures).

### High Compression and Flexible Implant Housing

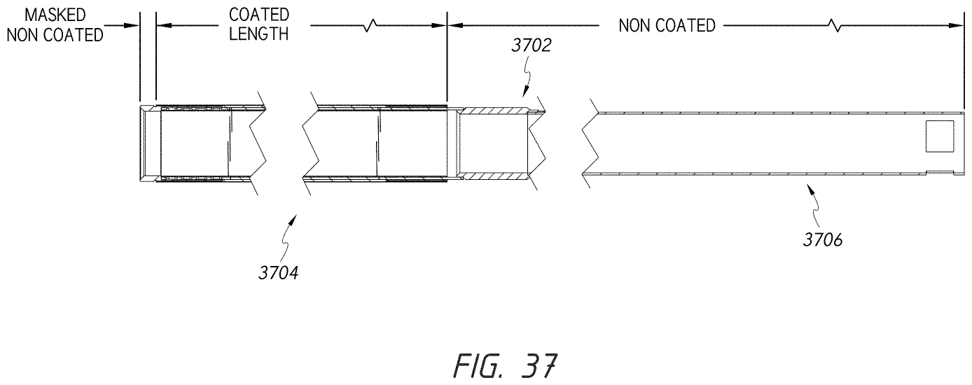

In some embodiments, the capsule 106 may have an alternate construction than discussed above, which are shown in FIGS. 37-39B. Valves and their location in the human anatomy are challenging to navigate in order to reposition or retrieve a replacement device (e.g., prosthesis or replacement valve 70), such as disclosed herein.

Advantageously, this alternative construction may allow for repositioning or retrieval of the implant (e.g., replacement heart valve). To do so, the capsule 3702 may be flexible, be good in compression, and good in tension. Thus, embodiments of the capsule 3702 may be universally flexible, able to deliver high compressive and tensile forces, and a low profile.

As shown in FIG. 37, the capsule 3702 can have a similar configuration to that of capsule 106 as disclosed above. The capsule 3702 can be formed of a distal section 3704 and a proximal section 3706. In some embodiments, the distal section 3704 has a larger diameter than the proximal section 3706. In some embodiments, the diameters may be the same.

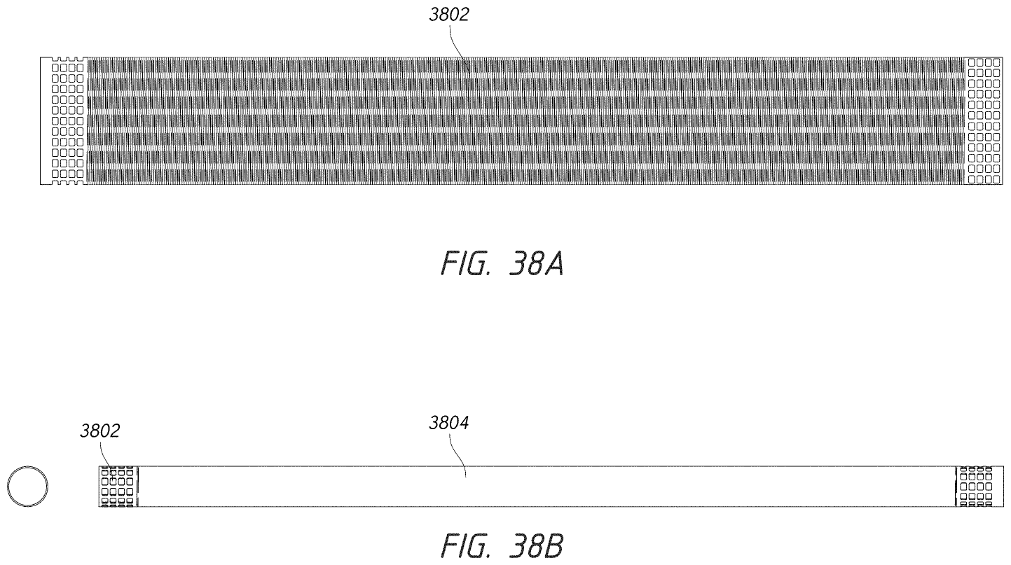

FIG. 38A illustrates a flat pattern of the distal section 3704. The distal section 3704 can be formed from a laser cut hypotube 3802. As discussed above, the metal component can be a laser cut hypotube with an interrupted spiral. The hypotube 3802 can include a number of cuts and window sizes, dictating the flexibility of the metal structure.

Further, the distal section 3704 can include an outer jacket 3804 which can partially or fully surround the hypotube 3802, as shown in FIG. 38B. For example, the jacket 3804 can cover greater than 70, 80, 90, or 95% of a length of the hypotube 3802. In some embodiments, thermoplastics may be used on the outer diameter of the hypotube 3802 to form the outer jacket 3804. An optional porous fluoropolymer can also be added to the outside, which is the “coated length” of FIG. 37. Advantageously, the metal structure of the hypotube 3802 can provide compression resistance, whereas the liner and/or jacket material 3804 can provide tension resistance.

Further, the hypotube 3802 can have an inner liner. In some embodiments, the inner liner is a fluoropolymer. In certain embodiments, the inner liner may be porous. The inner liner can then be bonded to a metal structure of the hypotube 3802, such as using a reflow process. A polymer can be used which bonds the inner liner to the metal. For example, a low durometer thermoplastic elastomer or a thermoplastic polyurethane can be used.

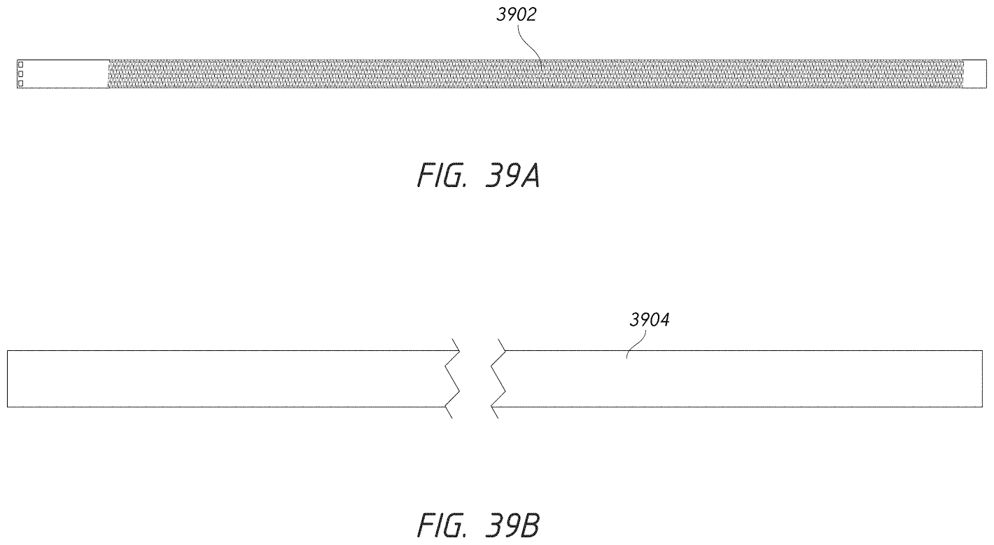

The proximal section 3706 can further include a lasercut hypotube 3902, shown in FIG. 39A which can be surrounded by an outer polymer jacket 3904, shown in FIG. 39B.

### Fail-Safe Knobs or Load Indication Knobs

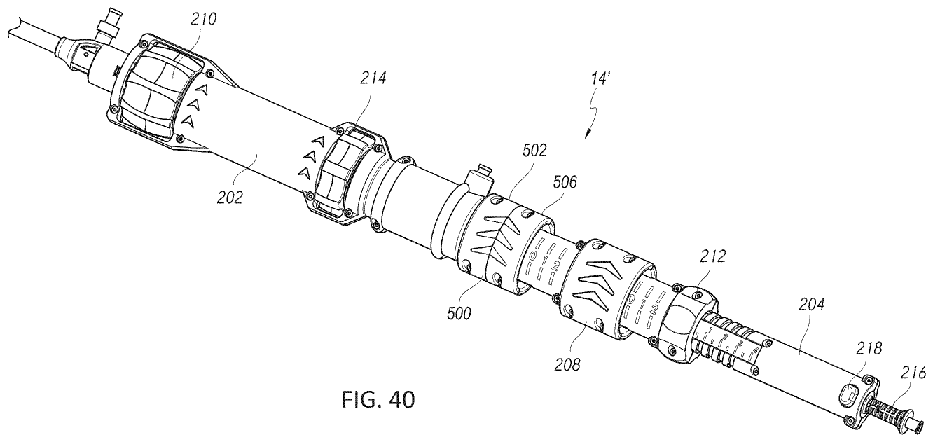

Another embodiment of the handle 14′ is shown in FIG. 40. The handle 14′ is similar to the handle 14 shown in FIGS. 16-17 except as described differently below. The handle 14′ can include one or more failsafe knobs 500. The failsafe knobs 500 may advantageously prevent the delivery system 10 from reaching a certain unacceptable or undesired level of force or load (e.g., shear force or bending force) that could result in device failure. In accordance with several embodiments, the failsafe knob 500 can act like a backup “fuse” that permanently or temporarily disables continued advancement of one or more lumens or shafts of the delivery system 10 once a threshold level of force or load is reached. As one example, the failsafe knob 500 can be integrated with the distal pull wire knob 206, the proximal pull wire knob 208, or each of the knobs 206, 208 such that the failsafe knob 500 can include the same functionality as the distal pull wire knob 206 or the proximal pull wire knob 208 described above in relation to FIGS. 16-17. In some embodiments, the failsafe knob 500 can be integrated with any knob on the handle 14′ (e.g., distal pull wire knob 206, proximal pull wire knob 208, outer sheath knob 210, depth knob 212, mid shaft knob 214, knob 216, or other knobs) or be a separate, standalone knob.

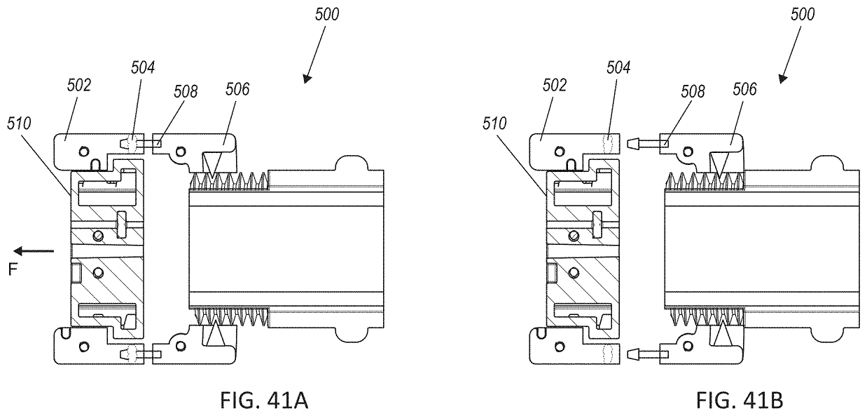

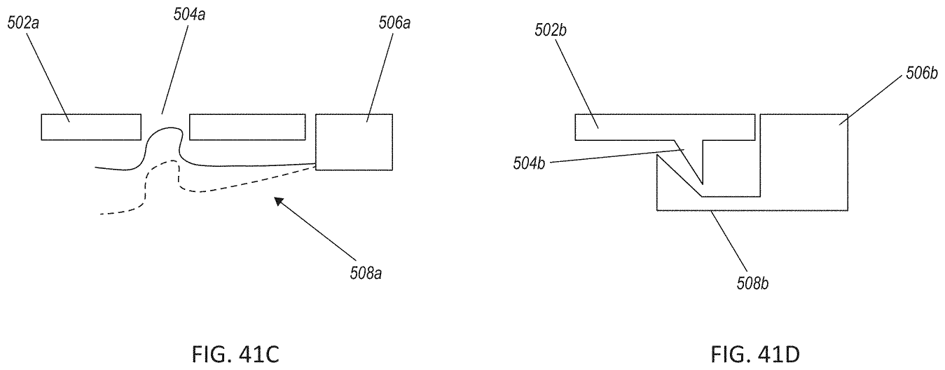

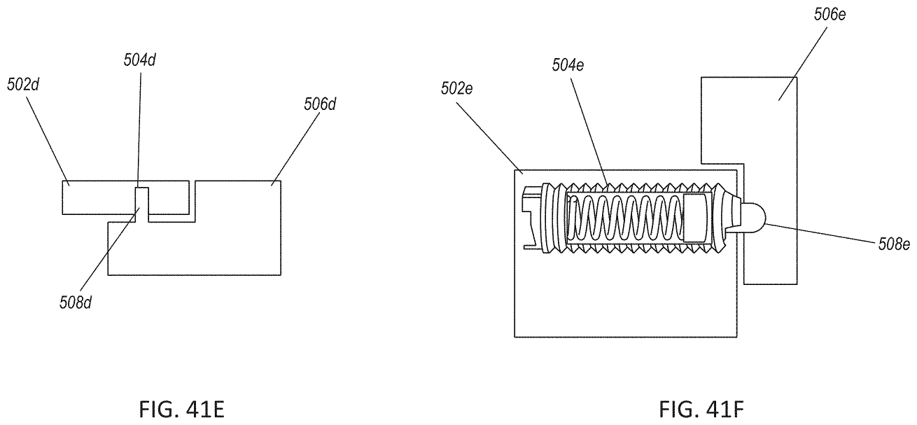

The failsafe knob 500 can be configured to decouple a lumen or shaft of the delivery system 10 from the handle 14′ when a threshold force is applied to the shaft assembly 12 (e.g., to one or more lumens or shafts) of the delivery system 10. For example, the failsafe knob 500 can include a first portion 502 and a second portion 506. In some embodiments, the first portion 502 can be positioned distally of the second portion 506 on the handle 14′. In a first configuration, as shown in FIG. 41A, the first and second portions 502, 506 can be coupled via, for example, a releasable coupling mechanism. In some configurations, the coupling mechanism can include a spring 504 and a projection 508. For example, the first portion 502 can include the spring 504 that can be configured to receive the projection 508 of the second portion 506. In some embodiments, the projection 508 can include one or more grooves configured to engage with the spring 504 when the first and second portions 502, 506 are coupled. In some configurations, the first portion 502 can include the projection 508 and the second portion 506 can include the spring 504. The first portion 502 can be configured to be detachable from the second portion 506. For example, in a second configuration, as shown in FIG. 41B, the first and second portions 502, 506 are decoupled. The spring 504 can comprise a nitinol spring or finger, a tab that deflects at a certain load, a custom spring, or other mechanisms. In some configurations, the coupling mechanism can be a quick connect coupler set or a quick disconnect coupler set. For example, the quick disconnect coupler set can be a quick disconnect hydraulic coupler set. The spring 504 can be a female component of the coupler set and the projection 508 can be a male component of the coupler set. The female component can be configured to releasably receive the male component. For example, the two components can disconnect when a threshold force exerted on the system 10 is reached, as further described below.

FIGS. 41C-41F are schematic illustrations for alternative coupling mechanisms. In some configurations, the coupling mechanism can include a spring tab, as shown in FIG. 41C. For example, one of the first portion 502a or the second portion 506a can include a spring tab 508a configured to engage with a hole or recess 504a of the other one of the first portion 502a or the second portion 506a when the first and second portions 502a, 506a are coupled. When a threshold force is exerted on the delivery system 10, as further described below, the spring tab 508a can deflect such that the first and second portions 502a, 506a decouple. The solid line in FIG. 41C illustrates the spring tab 508a in a first configuration being received in the hole 504a when the first and second portions 502a, 506a are coupled. The dashed lines in FIG. 41C illustrate the spring tab 508a in a second configurations when the first and second portions 502a, 506a decouple and the spring tab 508a deflects from the first configuration. In some configurations, the coupling mechanism can include a ratchet coupling, as shown in FIG. 41D. For example, one of the first or second portions 502b, 506b can include a ramped recess 508b and the other one of the first or second portions 502b, 506b can include a ramped protrusion 504b configured to be received by the ramped recess 508b when the first and second portions 502b, 506b are coupled. When the threshold force exerted on the system 10 is reached, the ramped protrusion 504b can deflect such that the first and second portions 502b, 506b decouple. In some configurations, the coupling mechanism can include a shear coupling, as shown in FIG. 41E. For example, one of the first or second portion 502d, 506d can include a shearing pin or other protrusion 508d configured to be received by a recess 504d in the other one of the first or second portion 502d, 506d. When the threshold force is exerted on the system 10, the shearing pin or other protrusion 508d can shear off or break such that the first and second portions 502d, 506d can be permanently decoupled. In some configurations, the coupling mechanism can include a spring plunger, as shown in FIG. 41F. For example, one of the first or second portions 502e, 506e can include the spring plunger and the other one of the first or second portion 502e, 506e can include a recess 508e configured to receive the pin or nose of the spring plunger 504e. When the threshold force is exerted on the system 10, the spring plunger 504e can decouple from the recess 508e such that the first and second portions 502e, 506e are decoupled.

The first portion 502 of the failsafe knob 500 can be coupled to an adapter 510 of or within the handle 14′. The adapter 510 can be positioned radially inward of the failsafe knob 500 and engage with the delivery housing 204. The second portion 506 of the failsafe knob 506 can include an engagement portion configured to engage with the delivery housing 204. For example, the delivery housing 204 can be threaded and the engagement portion can be a projection (e.g., a v-shaped projection or tab) configured to engage the threading in the delivery housing 204.

In use, a force F can be applied to the adapter 510 that can be correlated to a force exerted on the shaft assembly 12 (e.g., one or more lumens or shafts) of the delivery system 10 when the user navigates the shaft assembly 12 using the failsafe knob 500. When the force F reaches a threshold force, the first portion 502 may move distally until the projection 508 of the second portion 506 detaches from the spring 504 of the first portion 502. When the first portion 502 and the second portion 506 decouple, the user may no longer be able to rotate the failsafe knob 500 (either permanently or temporarily until the user reduces the force exerted on the shaft assembly 12 such that the force F is less than the threshold force). If temporary, once the force F is reduced, the user may couple the first portion 502 with the second portion 506 and resume the procedure. Advantageously, the failsafe knob 500 may prevent the delivery system 10 from failing due to excess forces or loads (e.g., (e.g., shear force or load) being exerted on the delivery system 10 (e.g., one or more shafts or lumens of the delivery system 10). The threshold force may be a level of force or load substantially below a calculated failure level during testing so that risk of failure is reduced. For example, if the one or more lumens or shafts of the shaft assembly 12 have an ultimate tensile strength of 20 lbs., the threshold force can be 10 lbs. for a safety factor of 2 or can be 15 lbs. for a safety factor of 1.5. In some configurations, the threshold force can be between about 1 lb. and about 50 lbs., about 10 lbs. and about 40 lbs., or about 20 lbs. and about 30 lbs. In some configurations, the threshold force can be configured for a safety factor of between about 1.0 and about 5.0, about 1.5 and about 4.5, about 2.0 and about 4.0, or about 2.5 and about 3.5.

In some embodiments, the second portion 506 can be positioned distally of the first portion 502 on the handle 14′. When the force F is applied to the adapter 510, the first portion 502 may move distally toward the second portion 506 such that the spring 504 is increasingly compressed. When the force F reaches the threshold force, the compressive forces may cause the first and second portions 502, 506 to decouple. Any of the releasable coupling mechanisms described herein can be configured to decouple the first and second portions 502, 506 based on the compressive forces applied to the first and second portions 502, 506.

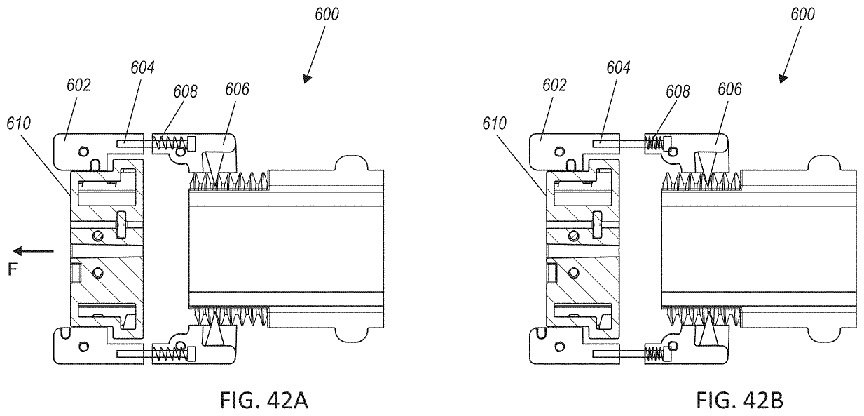

In some embodiments, a knob may not mechanically separate or decouple into two components but may instead provide a visual indicator of an amount of force being exerted on or by one or more shafts or lumens controlled by the knob. An embodiment of an indicator knob 600 is shown in FIGS. 42A-42B. The handle 14′ can include one or more indicator knobs 600 in addition to or instead of the failsafe knob 500. For example, the indicator knob 600 can be integrated with the distal pull wire knob 206, the proximal pull wire knob 208, or each of the knobs 206, 208 such that the indicator knob 600 can include the same functionality as the distal pull wire knob 206 or the proximal pull wire knob 208 described above in relation to FIGS. 16-17. In some embodiments, the indicator knob 600 can be integrated with any knob on the handle 14′ (e.g., distal pull wire knob 206, proximal pull wire knob 208, outer sheath knob 210, depth knob 212, mid shaft knob 214, knob 216, or other knobs) or be a separate, standalone knob. In some embodiments, the indicator knob 600 can be integrated with the failsafe knob 500.

The indicator knob 600 can be configured to indicate (e.g., visually) when a force being applied to the shaft assembly 12 (e.g., one or more lumens or shafts controlled by, or operatively coupled to, the indicator knob 600) of the delivery system 10 increases and/or decreases, a “real-time” amount of force being applied, or when a threshold amount of force has been reached. For example, the indicator knob 600 can include a first portion 602 and a second portion 606. The first portion 602 can be positioned distally from the second portion 606. The first portion 602 can be connected to the second portion 606 via a connector 604. A first or distal end of the connector 604 can be moveably secured in the first portion 602 and a second or proximal end of the connector 604 can be moveably secured in the second portion 606. For example, the first and second portions 602, 606 can have a recess (e.g., groove, notch, slot) that extends from an opening of the first portion 602 or the second portion 606. The recess of the first portion 602 can be configured to receive the first end and/or a distal portion of the connector 604. The recess of the second portion 606 can be configured to receive the second end and/or a proximal portion of the connector 604. The second portion 606 can include a spring 608 that surrounds the connector 604 within the second portion 606.

As shown in FIG. 42A, when the first and second portions 602, 606 are in a first configuration, the connector 604 can be substantially or entirely encompassed by the first and second portions 602, 606. Moreover, the second end of the connector 604 can be enlarged such that the second end has a greater diameter than the adjacent portions of the connector 604. For example, the enlarged second end can have a larger diameter than the opening of the recess of the second portion 606 such that the connector 604 may not be removed from the second portion 606. Moreover, the spring 608 can be positioned between the enlarged second end of the connector 604 and the opening of the second portion 606 to exert a force on the second portion 606 toward the first portion 602. When the first and second portions 602, 606 are in a second configuration, the connector 604 may be at least partially or substantially exposed such that a distance between the first and second portions 602, 606 is greater in the second configuration than in the first configuration. Moreover, the spring 608 can be compressed in the second configuration.

The first portion 602 of the indicator knob 600 can be coupled to an adapter 610 of or within the handle 14′. The adapter 610 can be positioned radially inward of the indicator knob 600 and engage with the delivery housing 204. The second portion 606 of the indicator knob 606 can include an engagement portion configured to engage with the delivery housing 204. For example, the delivery housing 204 can be threaded and the engagement portion can be a projection (e.g., a v-shaped projection) that can be configured to engage the threading in the delivery housing 204.

In use, a force F can be applied to the adapter 610 that is correlated to a force exerted on the shaft assembly 12 of the delivery system 10 when the user navigates the shaft assembly 12 (e.g., one or more shafts or lumens of the shaft assembly 12) using the indicator knob 600. When a force is exerted on the shaft assembly 12 (e.g., one or more shafts or lumens of the shaft assembly 12), the force F applied to the adapter 610 may cause the first portion 602 to move distally while the position of the second portion 604 can remain unchanged. In some embodiments, both portions 602, 606 can be configured to move in response to the applied force F. As the force exerted on the shaft assembly 12 (e.g., one or more shafts or lumens of the shaft assembly 12 operatively coupled to the indicator knob 600) increases, the increasing force F applied to the adapter 610 may cause the distance between the first and second portions 602, 606 to increase. The increasing force F may cause an increasing length of the connector 604 to be uncovered. The distance between the first and second portions 602, 606 and the length of the connector 604 that is uncovered can correlate (e.g., be proportional) with the amount of force F that is being exerted on the adapter 610. For example, the shorter the distance between the first and second portions 602, 606 and the greater length of the connector 604 that is covered, the less force F that may be applied to the adapter 610. Moreover, the greater the distance between the first and second portions 602, 606 and the greater length of the connector 604 is uncovered, the greater the force F that may be applied to the adapter 610. Accordingly, as the force F is reduced, the distance between the first and second portions 602, 606 and the length of the connector 604 that is uncovered may decrease. In some embodiments, the distance between the first and second portions 602, 606 may be inversely related to the force F being applied.

The connector 604 may include indicators along the length of the connector 604 that can correlate with the force F being applied to the delivery system 10. For example, the indicators can include colors, bands, stripes, numbers, shapes, symbols, or the like. The connector 604 can include any number of indicator(s). For example, the connector 604 can include one, two, three, four, five, six, or more than six indicator(s). In some embodiments, the connector 604 may include three indicators. The three indicators can include a band of different colors. For example, the three indicators can include one or more red bands, one or more yellow bands, and one or more green bands. The one or more green bands can include a single green band at or near the middle of the connector 604. The one or more red bands can include two red bands that can be positioned adjacent the first and second portions 602, 606. The one or more yellow bands can include two yellow bands that can be positioned between the green band and the red bands. The green band can indicate that the amount of force being applied to the delivery system 10 is within an acceptable range or a preferred range. The red bands can indicate that the amount of force being exerted on the delivery system 10 is in an unacceptable range and the user should reduce the amount of force. The yellow bands can indicate the amount of force being applied to the delivery system 10 is within an acceptable range but the amount of force is increasing to the unacceptable range or the amount of force is decreasing to the preferred range.

In some embodiments, the second portion 606 can be positioned distally of the first portion 602 on the handle 14′. The distance between the first and second portions 602, 606 may be inversely related to the force F being exerted on the delivery system 10. For example, the greater the distance between the first and second portions 602, 606 and the greater length of the connector 604 is uncovered, the less the force F that may be applied to the adapter 610. Accordingly, as the force F is increased, the distance between the first and second portions 602, 606 and the length of the connector 604 that is uncovered may decrease.

In some embodiments, the indicator knob 600 can include the same or similar functionality as the failsafe knob 500. For example, a distance between the first and second portions 602, 606 of the indicator knob 600 may increase as the force F being exerted on the delivery system 10 increases, as described above. Once the force F reaches a threshold force, the first and second portions 602, 606 may decouple.

From the foregoing description, it will be appreciated that an inventive product and approaches for implant delivery systems are disclosed. While several components, techniques and aspects have been described with a certain degree of particularity, it is manifest that many changes can be made in the specific designs, constructions and methodology herein above described without departing from the spirit and scope of this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A delivery system for a replacement heart valve, the delivery system comprising:
an inner shaft having a proximal end and a distal end,
wherein the distal end of the inner shaft comprises a manifold,
wherein the manifold comprises a plurality of circumferentially-spaced apart apertures, each of the apertures having a tooth extending from a distal edge of the aperture toward a proximal edge of the aperture;
an outer shaft member configured to cover the apertures of the manifold when in a distal position to prevent release of the looped end of the attachment tether,
wherein the outer shaft member comprises at least one aperture configured to be aligned with the plurality of circumferentially-spaced apart apertures of the manifold;
at least one attachment tether configured to releasably connect to at least one eyelet of the replacement heart valve,
wherein a first looped end of the at least one attachment tether is configured to be attached to the distal end of the inner shaft,
wherein a second looped end of the at least one attachment tether is configured to extend through the at least one eyelet of the replacement heart valve, through the at least one aperture of the outer sleeve into a respective one of the apertures of the manifold, and then looped over a free proximal end of the tooth,
wherein at least one of the outer shaft member and the inner manifold is configured to translate axially relative to the other to uncover the second looped end of the at least one attachment tether such that the second looped end can be removed from the tooth, thereby facilitating release of the replacement heart valve from the delivery system.

2. The delivery system of claim 1, wherein the at least one aperture of the outer shaft member comprises a plurality of circumferentially-spaced apart apertures.

3. The delivery system of claim 1, wherein the outer shaft member is configured to be moved proximally relative to the inner shaft to uncover the second looped end of the at least one attachment tether.

4. The delivery system of claim 1, wherein the inner shaft is configured to be moved distally relative to the outer shaft member to uncover the second looped end of the at least one attachment tether.

5. The delivery system of claim 1, wherein the outer shaft member comprises a sleeve.

6. The delivery system of claim 1, wherein the at least one attachment member comprises a plurality of attachment tethers, wherein a first looped end of each of the plurality of attachment tethers is configured to be attached to the distal end of the inner shaft, and wherein a second looped end of each of the plurality of attachment tethers is configured to configured to extend through the at least one eyelet of the replacement heart valve, through the at least one aperture of the outer sleeve into a respective one of the apertures of the manifold, and then looped over a free proximal end of the tooth of the respective one of the apertures of the manifold.

7. A method of facilitating delivery of a replacement heart valve within a body of a patient, the method comprising:
advancing a distal portion of a delivery system to a desired implantation location within a heart of the patient,
wherein the delivery system comprises an inner shaft and an outer shaft member,
wherein the distal end of the inner shaft comprises at least one radial aperture, the at least one radial aperture having a tooth extending from a distal edge of the at least one radial aperture toward a proximal edge of the at least one radial aperture,
wherein the outer shaft member comprises at least one radial aperture configured to be aligned with the at least one radial aperture of the inner shaft,
wherein a first looped end of an attachment tether is attached to a distal end of the inner shaft,
wherein a second looped end of the attachment tether is removably coupled to the tooth of the at least one radial aperture of the inner shaft after having been inserted through an eyelet of the replacement heart valve;
causing the outer shaft member and the inner shaft to transition from a locked configuration in which the second looped end of the attachment member cannot be removed from the tooth to an unlocked configuration in which the second looped end of the attachment member can be removed from the tooth, thereby decoupling the replacement heart valve from the delivery system and allowing the replacement heart valve to remain in the desired implantation location; and
removing the delivery system from the patient.

8. The method of claim 7, wherein the desired implantation location is a native mitral valve.

9. The method of claim 7, wherein the desired implantation location is a native tricuspid valve.

10. The method of claim 7, wherein causing the outer shaft member and the inner shaft to transition from the locked configuration to the unlocked configuration comprises moving the inner shaft in a distal direction.

11. The method of claim 7, wherein causing the outer shaft member and the inner shaft to transition from the locked configuration to the unlocked configuration comprises moving the outer shaft member in a proximal direction.

12. A delivery system for a replacement heart valve, the delivery system comprising:
a shaft having a proximal end and a distal end;
a manifold on a distal end of the shaft, wherein the manifold comprises a plurality of radially extending apertures, each aperture having a tooth;
at least one attachment tether configured to releasably connect to the replacement heart valve, wherein looped portions of the at least one attachment tether extend through the radially extending apertures of the manifold to surround the tooth; and
a sleeve configured to cover the radially extending apertures in a distal position to prevent release of the looped portions, wherein the sleeve is configured to be proximally translated to a proximal position to uncover the looped portions so that the replacement heart valve is released from the at least one attachment tether.

13. A delivery system for a replacement heart valve, the delivery system comprising:
a shaft having a proximal end and a distal end;
a manifold on a distal end of the shaft, wherein the manifold comprises a plurality of radially extending apertures;
at least one attachment tether configured to releasably connect to the replacement heart valve, wherein looped portions of the at least one attachment tether extend through the radially extending apertures of the manifold; and
a release tether configured to extend through the looped portions of the at least one attachment tether;
wherein when the release tether is withdrawn from the looped portions, the replacement heart valve is released from the at least one attachment tether.

14. The delivery system of claim 13, wherein the manifold comprises an inner manifold and an outer manifold.

15. The delivery system of claim 14, wherein the outer manifold comprises the radially extending apertures for receiving the looped portions of a distal end of the at least one attachment tether.

16. The delivery system of claim 15, wherein a proximal end of the at least one attachment tether is attached to the inner manifold.

17. The delivery system of claim 13, wherein the at least one attachment tether comprises one and only one attachment tether.

18. The delivery system of claim 13, wherein the at least one attachment tether comprises a plurality of attachment tethers.
